(19) United States
(12) Patent Application Publication
(10) Pub. No.: US 2024/0002679 A1
(43) Pub. Date: Jan. 4, 2024

(54) APPLICATIONS, METHODS, AND TOOLS FOR DEVELOPMENT, RAPID PREPARATION AND DEPOSITION OF A NANOCOMPOSITE COATING ON SURFACES FOR DIAGNOSTIC DEVICES INCLUDING ELECTROCHEMICAL SENSORS

(71) Applicant: PRESIDENT AND FELLOWS OF HARVARD COLLEGE, Cambridge, MA (US)

(72) Inventors: Pawan JOLLY, Cambridge, MA (US); Sanjay Sharma TIMILSINA, Cambridge, MA (US); Nolan DURR, Cambridge, MA (US); Donald E. INGBER, Cambridge, MA (US)

(73) Assignee: PRESIDENT AND FELLOWS OF HARVARD COLLEGE, Cambridge, MA (US)

(21) Appl. No.: 18/267,989

(22) PCT Filed: Nov. 29, 2021
(86) PCT No.: PCT/US2021/060960

§ 371 Date: Jun. 16, 2023

Related U.S. Application Data
(60) Provisional application No. 63/196,736, filed on Jun. 4, 2021.
(60) Provisional application No. 63/126,690, filed on Dec. 17, 2020.

(51) Int. Cl. C09D 5/16; G01N 33/543; C09D 5/24; C09D 7/61; C09D 189/00; G01N 27/414

(52) U.S. Cl. CPC C09D 5/1687; G01N 33/54373; G01N 33/54393; C09D 5/24; C09D 7/61; C09D 189/00; G01N 27/4145

(57) ABSTRACT

Method for making a coating on a surface of a substrate are described herein. The methods include applying a mixture to a surface of a substrate while maintaining the substrate at an elevated temperature. The mixture includes a particulate material and a proteinaceous material.

### CROSS-REFERENCE TO RELATED APPLICATIONS

This application claims benefit under 35 U.S.C. § 119(e) to U.S. Provisional Application No. 63/196,736, filed Jun. 4, 2021, and U.S. Provisional Application No. 63/126,690, filed Dec. 17, 2020, the contents of each of which are incorporated herein by reference in their entireties.

### FIELD OF THE INVENTION

This invention relates to compositions and methods for making anti-fouling and electrically responsive coatings. The rapid preparation of the coatings is also described.

### BACKGROUND

Molecular diagnostics and assays rely on the specific interaction between a capture agent and a target of interest. While selectivity is an inherent property of the capture agent's affinity for its target, non-specific interactions can considerably decrease the sensitivity of an assay and result in false positives.

Molecular blockers of varying molecular weight, including Bovine Serum Albumin (BSA), casein, pluronic acid, and poly(ethylene glycol) polymers (PEG) among others, have been used to limit non-specific binding interactions that may occur at surfaces and/or in solution. For example, the surface of microtiter plates used in enzyme linked sandwich immunoassays (ELISAs) are typically blocked with BSA to reduce non-specific adsorption of proteins at their surface and BSA is also usually added to the buffers used during the assay.

For assays based on a final optical readout (e.g. absorbance, fluorescence, chemiluminescence or electrochemiluminescence), the blockers do not interfere with the final measurement. This is because the assay chemistry and measurements are fully decoupled. The assay is carried out on a surface (e.g. plates, microbeads and nanoparticles) whereas the final measurement is performed using an external transducer. For example, in fluorescence based assays, light of a predetermined wavelength is shined on a surface bearing the capture agent and the light emitted is quantified by a photodiode or CCD sensor (i.e., the transducer). In the forgoing example, the surface where the molecular interaction takes place acts as a passive support and does not contribute to the measurement.

A more challenging situation is presented when an electrochemical read out is desired since the assay is carried out on the transducer surface. The capture agent is typically immobilized at the surface of the transducer using strategies that should maximize its density and orientation, prevent non-specific interactions, and at the same time, preserve the ability of the electrode to record electrochemical signals with high sensitivity. Accordingly, it is imperative to the development of these electrochemical sensors that its surface resists biofouling, the aggregation of biomolecules on the surface, and that it maintains its initial physical and/or chemical properties (e.g. conductivity).

One of the problems encountered in providing these anti-fouling coatings is deposition and attachment on various substrates. For example, gold is a good electrode coating substrate that has a high affinity to proteins due to thiol-gold interactions. However other surfaces can be more challenging.

Furthermore, the standard process to functionalize transducers with an anti-fouling coating is to drop cast a composition on the surface of the transducer and incubating overnight. This step adds a day to the process and thus lengthens the time to make the final sensors. Integration into a multi-step on continuous manufacturing process may be frustrated, since the coating step may constitute a bottleneck to the entire process.

There is therefore a need for coatings and methods for preparing these coating on various electrical transducers that can accommodate capture agents, prevent non-specific interaction and preserve the ability of the electrical transducer to record electrochemical signals with high sensitivity. The present disclosure addresses some of these needs.

### SUMMARY

In general, the inventions described herein relate to compositions and their application to transducer surfaces. The coatings protect these surfaces from unwanted interactions that impede or diminish their intended function. For example, the coatings can be applied to electrodes and gates of bio-Field Effect Transistors (bio-FET), providing electrical transducers that can be utilized in complex matrices such as blood and plasma. Furthermore, implementations described herein are amenable to continuous processes such as reel to reel manufacturing, allowing large scale commercialization of devices using the coatings.

According to some implementations of the present disclosure, a method for making a coating on a surface of a substrate includes applying a mixture to a surface of a substrate while maintaining the substrate at an elevated temperature, wherein the mixture comprises a particulate material and a proteinaceous material. Optionally, the mixture further comprises a cross-linking agent. For example, the proteinaceous material includes a cross linking agent attached to or as part of the proteinaceous material's structure. Optionally, the elevated temperature is maintained for at least 10 seconds and less than two minutes. Optionally, the elevated temperature is at least 50° C. Optionally, the method further comprises denaturing the proteinaceous material. For example, optionally said denaturing the proteinaceous material is: prior to mixing the proteinaceous material with the particulate material; and/or after applying the mixture to the substrate.

In some implementations of the method, the substrate is particle, a nano-particle, a micro-particle, a nano-fiber, a micro-fiber, a flake, a chip, a crystal, a porous substrate, a wafer, a wire, a nano-wire, a micro-wire, a channel, a nano-channel, a micro-channel, a rod, a nano-rod, a micro-rod, a foil, a sheet, a web, or combination of these forms. Optionally, the substrate includes a material selected from the group consisting of metals, polymers, carbon based materials, ceramics, glass and any combinations thereof. Optionally, the substrate includes gold. Optionally, the substrate includes graphite, diamond, glassy carbon, or carbon nano-tubes. Optionally, the substrate includes an organic polymer.

In some implementations of the method, the particulate material is a rod, fiber, a particle, a flake or combinations of these. Optionally, the particulate material is a dielectric. Optionally, the particulate material is a conductor or semi-conductor. Optionally, the particulate material comprises an allotrope of carbon atoms arranged in a hexagonal lattice.

Optionally, the method includes applying a layer of a second substrate on the coating of proteinaceous material and optionally coating a second mixture comprising a second mixture and second proteinaceous material on the second substrate, providing a layered material having alternating layers of substrate and proteinaceous/particulate material.

In some implementations, the method, further comprises a step of pre-treating the substrate prior to applying the mixture. Optionally, applying the mixture comprises spraying, spin coating, dip coating, inkjet printing, 3-D printing, vapor deposition, painting, and/or drop casting. Optionally, the method is a continuous process or semi-continuous process.

In some implementations, the method further comprises denaturing the proteinaceous material and subsequently adding a temperature sensitive material to the mixture prior to coating the substrate. In some implementations, the substrate surface defines a channel or chamber, such as a channel in or chamber in a microfluidic device. In some implementations, the substrate is a micro/nano gap devices where the coating can enable higher sensitivity and the coating can either be used to coat the surfaces of a gap in the micro/nano gap device, or the coating is applied for surface modification to enable linking of specific probes and antifouling properties.

According to some implementations of the present disclosure, a substrate including a coating on a surface thereof, wherein said coating is applied using a method described herein. Optionally, the substrate is an electrode, a capacitor, a bio-Field Effect Transistor (bio-FET), a transistor, or an optical device.

According to some implementations of the present disclosure, a capacitor including a dielectric material dispersed in a denatured and cross-linked proteinaceous material, and covering a conductive substrate.

According to some other implementations of the present disclosure, a bio-FET comprising a composition including a particulate material dispersed in a denatured proteinaceous material and coating at least a portion of a transistor. Optionally, the compositing is coated on a gate of the bio-FET.

The above summary is not intended to represent each implementation or every aspect of the present disclosure. Additional features and benefits of the present disclosure are apparent from the detailed description and figures set forth below

### BRIEF DESCRIPTION OF THE FIGURES

This patent or application file contains at least one drawing executed in color. Copies of this patent or patent application publication with color drawings will be provided by the Office upon request and payment of the necessary fee.

FIG. 1 depicts a cyclic voltammetry measurement of the biosensor made using different coating deposition times, according to some implementations.

FIG. 2 is a plot of BSA concentration data for an anti-fouling coating, according to some implementations.

FIG. 3 is a plot of glutaraldehyde concentration data for an anti-fouling coating, according to some implementations.

FIG. 4 is a plot of a functionalized graphene oxide concentration data for an anti-fouling coating, according to some implementations.

FIG. 5 is a plot comparing coating methods, according to some implementations.

FIG. 6 is a plot comparing coating methods with a NT-proBNP assay, according to some implementations.

FIG. 7 shows a 3D schematic of and electrode with antifouling nanocomposite, according to some implementations.

FIG. 8 is a plot showing electrochemical characterization of a coating, according to some implementations.

FIG. 9A and FIG. 9B are CV plots showing oxidation and reduction peaks of ferri-/ferrocyanide, according to some implementations. (FIG. 9A) dipping in water at room temperature and, (FIG. 9B) cooling to room temperature before dipping.

FIG. 10 is a plot showing optimization of coating time for sensors, according to some implementations.

FIG. 11 is a plot showing optimization of a coating including reduced Graphene oxide (rGOx), according to some implementations.

FIG. 12 is a plot showing optimization of BSA for a coating composition, according to some implementations.

FIG. 13A is an optimization plot for rGOx, according to some implementations.

FIG. 13B shows a Cyclic Voltammogram (CV) showing oxidation and reduction peaks of ferri-/ferrocyanide of various nanocomposite-coated electrodes, according to some implementations

FIG. 14 is a plot showing electrochemical characterization of various electrode coatings, according to some implementations.

FIG. 15 shows CVs of bare gold- (left) and coated gold electrodes (right) of an of ferri-/ferrocyanide at different scan rates, according to some implementations.

FIG. 16 shows a plot of extracted oxidation/reduction peak current (ip) mean values derived from the CV scans shown in FIG. 15 plotted versus the square root of the scan rate, according to some implementations.

FIG. 17 shows a plotted comparison of antifouling activity with mean value of current density recorded at bare gold electrodes and Gold electrodes with antifouling coating stored for 9 weeks at 4° C. in 1% BSA and unprocessed human plasma, according to some implementations.

FIG. 18A and FIG. 18B are plots characterizing the antifouling nanocomposite coating, according to some implementations. Bar graph (FIG. 18A) shows current density of fresh sensors (black bar) and sensors after 1 hour in varying biofluids: whole blood, saliva, and urine (grey bar). UV absorption spectra (FIG. 18B) of BSA when mixed with/without GA and/or GOx. a.u., arbitrary units.

FIG. 19A-19E are scanning electron micrograph of the bare Gold (FIG. 19A, FIG. 19D), Gold/BSA/GOx (FIG. 19B), and Gold/BSA/GOx/GA (anti-fouling coating) (FIG. 19C, FIG. 19E), according to some implementations.

FIG. 20A shows a 3D image of roughness for a bare gold electrode, and FIG. 20B shows a 3D image of coated gold electrode, according to some implementations.

FIG. 20C-20H show TEM images of bare gold (FIG. 20C, FIG. 20F), coated gold electrode with top Iridium layer (FIG. 20D, FIG. 20G), and without top Iridium layer (FIG. 20E, FIG. 20H) for better contrast.

FIG. 21A-21B show AFM 2D (FIG. 21A) and 3D (FIG. 21B) survey images of bare gold electrode, according to some implementations.

FIG. 21C-21D show AFM 2D (FIG. 21C) and 3D (FIG. 21D) survey images of gold electrode with antifouling coating, according to some implementations.

FIG. 22 shows an XPS spectra for gold electrode coated with antifouling coating, according to some implementations.

FIG. 23A-23L depict the characterization of the antifouling nanocomposite coating. X-ray Photoelectron Spectroscopy (XPS) spectrum of the gold electrode with antifouling coating for C1s peak (FIG. 23A), O1s peak (FIG. 23B), N1s peak (FIG. 23C), and Au4f peak (FIG. 23D). Time-of-Flight Secondary Ion Mass Spectrometry (TOF-SIMS) spectra of the gold electrode with antifouling coating for positive ions (FIG. 23E) and (FIG. 23F) and negative ions (FIG. 23G) and (FIG. 23H). UV absorption spectra of denatured BSA when mixed with/without GA and/or GOx. a.u., arbitrary units (FIG. 23I). Contact angle of the EC-Biosensor before and after cleaning and plasma treatment with different kinds of coatings (FIG. 23J). Contact angle measurement for cleaned Gold chips (FIG. 23K) and Gold chip with the anti-fouling coating (FIG. 23L).

FIG. 24A-24G show schematics and calibration curves for MI and TBI biomarkers using EC-biosensors and 96 well plate. FIG. 24A shows a schematic for the preparation and assay steps for the EC-Biosensor. FIG. 24B-24G are calibration curves for different biomarkers including (24B) cardiac troponin I (cTnI); (24C) B-type natriuretic peptide (BNP); (24D) N-terminal (NT)-pro hormone BNP (NT-proBNP); (24E) cardiac troponin ITC complex (cTnITC); and (24F) Glial fibrillary acidic protein (GFAP).

FIG. 25A-25F are images of a drop on a chip for contact angle measurements. FIG. 25A is an uncleaned chip with protective organic layer. FIG. 25B is a chip cleaned with acetone and Isopropyl alcohol. FIG. 25C is a plasma treated chip. FIG. 25D is a plasma-treated chips with BSA coating. FIG. 25E is a plasma-treated chip with BSA and GOx coating. FIG. 25F is a plasma-treated chips with antifouling nanocomposite coating.

FIG. 26A-26C illustrates optimization and two-step assay for different biomarkers. FIG. 26A shows optimization of detection antibody for the assay of cTnI. FIG. 26B is a calibration curve of cTnI. FIG. 26C is a calibration curve of NT-proBNP.

FIG. 27A-27G illustrates assay development and optimization for single step assay for different biomarkers. FIG. 27A shows optimization of cTnI capture antibody. FIG. 27B shows optimization of cTnI detection antibody. FIG. 27C shows optimization of HRP-Streptavidin. FIG. 27D shows optimization of TMB incubation time. FIG. 27E shows optimization of BNP detection antibody. FIG. 27F shows optimization of NT-proBNP detection antibody. FIG. 27G shows optimization of cTnITC detection antibody.

FIG. 27H-27J illustrate cross-reactivity test of the EC Biosensor. CV oxidation and reduction peaks of uric acid (FIG. 27H), Dopamine (FIG. 27I), and Tryptophan (FIG. 27J) are depicted.

FIG. 28A-28F shows characterization and stability of antifouling nanocomposite and precipitated TMB. FIG. 28A shows an assay of NT-proBNP. FIG. 28B depicts a comparison of rapid versus 24 h coating for assay of cTnI. FIG. 28C shows stability of precipitated TMB for detection of cTnITC. FIG. 28D shows stability of precipitated TMB for detection of GFAP. FIG. 28E shows stability of coating solution stored at 4 degrees. FIG. 28F shows stability of coating solution stored at room temperature.

FIG. 29A-29E illustrates the specificity and Multiplexed detection for MI and TBI Biomarkers using EC-Biosensors. FIG. 29A shows a schematic for multiplexed detection on the EC-Biosensor showing detection of four biomarkers in a single EC-Biosensor. FIG. 29B shows specificity and cross-reactivity of BNP antigen against different capture antibodies (anti-cTnITC, anti-NT-proBNP, anti-GFAP, and anti-S-100b) and detection antibodies (anti-cTnITC, anti-NT-proBNP, anti-GFAP, and anti-S-100b) along with specific detection with anti-BNP capture and detection antibody at different concentrations of BNP done in 96 well plate. FIG. 29C shows a calibration curve for multiplex detection of cTnITC on the EC-Biosensor with four different capture antibodies on each electrode (anti-cTnITC, anti-S-100b, anti-GFAP, and anti-NT-proBNP).

FIG. 29D shows a calibration curve for multiplex detection of increasing concentrations of cTnITC (left y-axis) and decreasing concentrations of GFAP (right y-axis) on the EC-Biosensor with four different capture antibodies on each electrode. FIG. 29E shows a calibration curve for multiplex detection of increasing concentrations of cTnITC and S-100b (left y-axis) and decreasing concentrations of GFAP and NT-proBNP (right y-axis) on the EC-Biosensor with four different capture antibodies on each electrode.

FIG. 30A-30H illustrates microfluidic integration and clinical validation of the assay. FIG. 30A is a picture of a microfluidic device where six EC-Biosensors can be placed to run the assay in parallel. FIG. 30B is a 3D schematic of the microfluidic channels and their interface with the EC-Biosensor. FIG. 30C shows a calibration curve for the assay of cTnITC performed on the microfluidic platform with reduced assay time using spiked plasma samples. FIG. 30D shows a calibration curve for the assay of GFAP performed on the microfluidic platform using spiked plasma samples. FIGS. 30E and 30F are plot showing validation of the EC Biosensor using standard 96 well assay for the detection of cTnITC (FIG. 30E) and GFAP (FIG. 30G). Bland-Altman plot for validation of EC Biosensor using clinical sample for cTnITC is shown by FIG. 30F and for GFAP is shown by FIG. 30H.

FIG. 31A-31F illustrates an assay of different biomarkers on EC-Biosensor. TMB oxidation and reduction peaks obtained with a CV on EC Biosensors with antifouling coating for detection of different concentrations of biomarkers of Myocardial infarction and Traumatic Brain Injury. FIG. 31A shows BNP, FIG. 31B shows NT-proBNP, FIG. 31C shows cTnI, FIG. 31D shows cTnITC, FIG. 31E shows GFAP, and FIG. 31F shows S100b.

FIG. 32A depicts a calibration curve of cTnITC; FIG. 32B depicts a calibration curve of GFAP, and FIG. 32C depicts multiplex detection of cTnITC and GFAP on EC Biosensor with four different capture antibodies on each electrode.

FIG. 33A-33C illustrate the specificity and cross-reactivity test for different biomarkers of MI and TBI done in 96 well plate. FIG. 33A shows the specificity and cross-reactivity of NT-proBNP antigen against different capture antibody and detection antibody. FIG. 33B shows the specificity and cross-reactivity of GFAP antigen against different capture antibody and detection antibody. FIG. 33C shows the specificity and cross-reactivity of S-100b antigen against different capture antibodies and detection antibody.

FIG. 34A-34D illustrates a specificity and cross-reactivity test for different Troponin antibody pair and antigen done in 96 well plate. FIG. 34A shows specificity and cross-reactivity of cTnITC antigen against different capture antibody and detection antibodies. FIG. 34B shows specificity and cross-reactivity of cTnI antigen against different capture antibodies and detection antibodies. FIG. 34C shows specificity and cross-reactivity of cTnITC antigen against different capture antibody and detection antibody. FIG. 34D shows specificity and cross-reactivity of cTnI antigen against different capture antibodies and detection antibodies.

### DETAILED DESCRIPTION

The methods, compositions and structures provided herein are based in part on the preparation of protective coatings on substrate surfaces. The coatings include conductive or non-conductive particulate materials in a proteinaceous matrix that can be rapidly formed on surfaces. In some examples, the proteinaceous material is rapidly denatured and cross-linked, forming a robust protective coating on electric transducers or forming a part of an electric transducer. In some implementations, the methods and compositions can be implemented for large scale manufacturing as well as small laboratory scale and rapid small scale prototyping.

According to some implementation, the invention includes the preparation of an electrochemically active surface blocker that can prevent non-specific interaction while keeping an electric transducer active. As used here an “electric transducer” is a device that interacts with a molecule, polymer, biological materials to provide an electrical signal. The signal can include, for example, a change in current, a change in voltage, a change in capacitance, a change in impedance, or a change in dielectric constant. In some implementations, the electric transducer is a biotransducer, such as an electrochemical transducer, an optical transducer, a bio-FET, or a piezoelectric biotransducer. In some implementations, the electric transducer is an electrode.

In some implementations, the methods include coating a surface of a substrate while maintaining the surface of the substrate at an elevated temperature. As used herein, an “elevated temperature” is a temperature above the ambient temperature, such as above room temperature. For example, the temperature can be above about 50° C. (e.g. above, 60, 70, 80, 90 or 100° C.). In some implementations, the elevated temperature is below about 180° C. (e.g., below about, 140, 130, 120, 110, or 100° C.). In some implementations, the elevated temperature is between about 60 and 110° C., between about 70 and 100° C., between about 80 and 90° C., such as about 85° C.

The temperature can be maintained by any method. For example, in some implementations, the substrate is place on a hot temperature controlled surface such as a hot plate. In some implementations, the heating is provided in an oven or hot air. In some implementations, UV light is used for heating the substrate directly on the surface to be coated, or on an opposite surface or area of the substrate. In some implementations, the substrate is immersed in a heated solution, such as including the coating composition.

The temperature is maintained for any amount of time to provide a robust surface coating. For example, in some implementations, the temperature is maintained for less than about 24 hours, less than about 12 hours, less than about 6 hours, less than about one hour, less than about minutes, less than about 10 minutes, less than about two minutes, or less than about a minute. In some implementations, the temperature is maintained for at least about 1 second, at least about seconds, at least about 10 seconds, at least about 30 seconds, at least about 40 seconds, at least about 45 seconds.

According to some implementations, the method includes coating the surface of the substrate with a mixture that includes a particulate material and a proteinaceous material. In some implementations, the proteinaceous material includes a cross-linking agent attached to or as part of the structure of the proteinaceous material. In some implementations, a cross-linking agent is added to the mixture. Without ascribing to a specific mechanism, the elevated temperature and time is used at least in part to modulate the amount of cross linking that occurs. The heating can also denature the proteinaceous material.

In some implementation, the mixture is heated to an elevated temperature prior to coating on the surface. In some implementations, the mixture is heated to an elevated temperature that is different than the elevated temperature to which the substrate is heated. In addition to heating, in some implementations, the mixture is homogenized, before or after it is heated. For example, the mixture can be sonicated before addition to the substrate.

As used herein, “to cross link” means to form one or more bonds between polymer chains so as to form a network structure such as a gel or hydrogel. The polymers are then “cross-linked” polymers. The bonding can be through hydrogen bonding, covalent bonding or electrostatic. The “cross linking agent” can be a bridging molecule or ion, or it can be a reactive species such as an acid, a base or a radical producing agent.

For molecular cross linking agents, the cross linking agents contain at least two reactive groups that are reactive towards numerous groups, including primary amines, carboxyls, sulfhydryls, carbohydrates and carboxylic acids. Proteins and peptide molecules have many of these functional groups and therefore proteins and peptides can be readily conjugated and cross-linked using these cross linking agents. Cross linking agents can be homobifunctional, having two reactive ends that are identical, or heterobifunctional, having two different reactive ends. In some embodiments the cross linking agent is a molecule such as glutaraldehyde, dimethyl adipimidate (DMA), dimethyl suberimidate (DMS), Bissulfosuccinimidyl suberate, formaldehyde, p-azidobenzoyl hydrazide; n-5-azido-2-nitrobenzoyloxysuccinimide; n-[4-(p-azidosalicylamido)butyl]-3′-(2′-pyridyldithio) propionamide; p-azidophenyl glyoxal monohydrate; bis [b-(4-azidosalicylamido)ethyl]disulfide; bis [2-(succinimidooxycarbonyloxy)ethyl] sulfone; 1,4-di [3′-(2′-pyridyldithio)propionamido] butane; dithiobis(succinimidyl propionate); disuccinimidyl suberate; disuccinimidyl tartrate; 3,3′-dithiobis(sulfosuccinimidyl propionate); 3,3′-dithiobis(sulfosuccinimidyl propionate) 1-ethyl-3-[3-dimethylaminopropyl] carbodiimide hydrochloride; Ethylene Gly col bi s (succinimidyl succinate); N-(E-maleimidocaproic acid hydrazide); [N-(E-maleimidocaproyloxy)-succinimide ester]; N-Maleimidobutyryloxysuccinimide ester; Hydroxylamine.HCl; Maleimide-PE G-succinimidyl carboxy methyl; m-Maleimidobenzoyl-N-hydroxysuccinimide Ester; N-Hydroxysuccinimidyl-4-azidosalicylic acid; N-(p-Maleimidophenyl isocyanate); N-Succinimidyl(4-iodoacetyl) Aminobenzoate; Succinimidyl-4-(N-maleimidomethyl)cyclohexane-1-carboxylate; Succinimidyl 4-(p-maleimidophenyl) Butyrate; Sulfo Disulfosuccinimidyl Tartrate; [N-(E-maleimidocaproyloxy)-sulfo succinimide ester; N-Maleimidobutyryloxysulfosuccinimide ester; N-Hydroxysulfosuccinimidyl-4-azidobenzoate; m-Maleimidobenzoyl-N-hydroxysulfosuccinimide Ester; Sulfosuccinimidyl (4-azidophenyl)-1,3 dithio propionate; Sulfosuccinimidyl 2-(m-azido-o-nitrobenzamido)-ethyl-1,3′-dithio propionate; Sulfosuccinimidyl 6-(4′-azido-2′-nitrophenylamino) hexanoate; Sulfosuccinimidyl-2-(p-azidosalicylamido)ethyl-1,3-dithiopropionate; N-(Sulfosuccinimidyl(4-iodoacetyl)Aminobenzoate); Sulfosuccinimidyl-4-(N-maleimidomethyl)cyclohexane-1-carboxylate; Sulfo succinimidyl 4-(p-maleimidophenyl) Butyrate; and mixtures of these. In some embodiments the cross linking agent is mone- or poly-ethylene glycol diglycidil ether. In some embodiments the cross linker is a homobifunctional cross linking agent such as glutaraldehyde.

As used herein “proteinaceous” material includes proteins and peptides, functionalized proteins, copolymers including proteins, natural and synthetic variants of these, and mixtures of these. For example, proteinaceous material can be Bovine Serum Albumin (BSA).

According to some implementations of the disclosure, the proteinaceous material can include or be replace with any blocking agent. As used herein a “blocking agent” or “molecular blockers” are compounds used to prevent non-specific interactions. The blocking agent when coated on the substrate surface prevents non-specific interactions or fouling of the surface when it is contacted or immersed in a complex matrix. The surface can include a capture agent, for example, directly attached to the surface or attached to the blocking agent. Non-specific interactions can include any interaction that is not desired between the target molecule and the surface, or between other components in solution. The blocking agent can be a protein, mixture of proteins, fragments of proteins, peptides or other compounds that can absorb to the surface of the substrate. For example, proteins (e.g., BSA and Casein), poloxamers (e.g., pluronics), PEG-based polymers and oligomers (e.g., diethylene glycol dimethyl ether), cationic surfactants (e.g., DOTAP, DOPE, DOTMA). Some other examples include commercially available blocking agent or components therein that are available from, for example, Rockland Inc. (Limeric, PA) such as: BBS Fish Gel Concentrate; PBS Fish Gel Concentrate; TBS Fish Gel Concentrate; Blocking Buffer for Fluorescent Western Blotting; BLOTTO; Bovine Serum Albumin (BSA); ELISA Microwell Blocking Buffer; Goat Serum; IPTG (isopropyl beta-D-thiogalactoside) Inducer; Normal Goat Serum (NGS); Normal Rabbit Serum; Normal Rat Serum; Normal Horse Serum; Normal Sheep Serum; Nitrophenyl phosphate buffer (NPP); and REVITABLOT™ Western Blot Stripping Buffer.

As used herein, “denaturing” is the process of modifying the quaternary, tertiary and secondary molecular structure of a protein from its natural, original or native state. For example, such as by breaking weak bonds (e.g., hydrogen bonds), which are responsible for the highly ordered structure of the protein in its natural state. The process can be accomplished by, for example: physical means, such as by heating, sonication or shearing; by chemical means such as acid, alkali, inorganic salts and organic solvents (e.g., alcohols, acetone or chloroform); and by radiation. A denatured protein, such as an enzyme, losses its original biological activity. In some instances, the denaturing process is reversible, such that the protein molecular structure is regained by the re-forming of the original bonding interactions at least to the degree that the original biological function of the protein is restored. In other instances, the denaturing process is irreversible or non-reversible, such that the original and biological function of the protein is not restored. Cross-linking, for example after denaturing, can reduce or eliminate the reversibility of the denaturing process.

The degree of denaturing can be expressed as a percent of protein molecules that have been denatured, such as a mole percent. Some methods of denaturing can be more efficient than others. For example, under some conditions, sonication applied to BSA can denature about of the protein and the denaturing is reversible. When BSA is denatured it undergoes two structural stages. The first stage is reversible whilst the second stage is irreversible (e.g., non-reversible) but does not necessarily result in a complete destruction of the ordered structure. For example, heating up to 65° C. can be regarded as the first stage, with subsequent heating above that as the second stage. At higher temperatures, further transformations are seen. In some embodiments, BSA is denatured by heating above about 65° C. (e.g., above about 70° C., above about above about 90° C., above about 100° C., above about 110° C., above about 120° C.), below about 200° C. (below about 190° C., 180° C., 170° C., 160° C., 150° C.), and for at least about 1 minute (e.g., at least about 2, 3, 4, 5, 10 or 20 minutes) but less than about 24 hours (e.g., less than about 12, 10, 8, 6, 4, 2 1 hour). According to some implementations, any ranges herein described, for example heating at about 90° C. but below about 150° C. and for at least about 1 minute but less than one hour. As previously noted, the heating can include be included as a separate step to heating of the substrate and include different temperature ranges and heating times.

In some implementations, denaturing of the proteinaceous material can occur before deposition on the substrate surface. In some implementations, denaturing can occur only upon deposition on the substrate surface, for example when only a heating step to heat the substrate is included. In some implementations, denaturing occurs before and after deposition, for example, where heating occurs before and after deposition of the mixture on the substrate surface.

In some embodiments the proteinaceous material used in the compositions and structures described herein are at least about 20% to about 100% (e.g., 20%, 30%, 40%, 50%, 60%, 70%, 80%, 90%, or 100%) denatured. In some embodiments, less than 50% of the denatured protein reverts back to its natural state (e.g., less than 40%, less than 30%, less than 20%, less than 10%, less than 1%). Therefore, the reversibility of the denaturing can be described as being 50% reversible, 40% reversible (60% irreversible), 30% reversible (70% irreversible), 20% reversible (80% irreversible), 10% reversible (90% irreversible) or even 0% reversible (100% irreversible).

In some implementations, an agent is added after the denaturing and/or cross linking steps. For example, in some implementations, an agent is temperature sensitive but is needed in the surface coating. For example, where the agent is a capture agent such as an enzyme or antibody that loses activity upon heating. In some implementations, the particulate material is added after the denaturing and/or cross linking step. For example, where the particulate material is a temperature sensitive material or nano-particle such as polymers (e.g., polypyrrol, agarose, or a gel).

The substrate can be in any form having a surface that can be coated. For example, the substrate can be included in the form of a particle, a nano-particle, a micro-particle, a nano-fiber, a micro-fiber, a flake, a chip, a crystal, a porous substrate, a wafer, a wire, a nano-wire, a micro-wire, a channel, a nano-channel, a micro-channel, a rod, a nano-rod, a micro-rod, a foil, a sheet, a web, or combination of these forms. In some implementations, the substrate is part of a microfluidic device, such as a channel or chamber therein. In some implementations, the substrate is part of a micro well plate. In some implementation, the substrate is an optical fiber. In some implementations, the substrate is part of a mico or nano-gap device.

In some implementations, the substrate can be a transparent material, an opaque material, an insulator, a conductor, a semi-conductor, a dielectric material, or include a combination of these properties. For example, in some implementations, the substrate can be a transparent conductor such as indium tin oxide (ITO). In some implementations, the substrate is polycrystalline silicon, single crystal silicon, a doped silicon, a silicon oxide, a silicon nitride, a silicon oxnitride, a metal oxide, a metal nitride, or a metal oxynitride.

In some implementations, the substrate includes a metal, a metalloid, a polymer, a ceramic, a glassy material, an amorphous material, a biological membrane, a carbon based material, or any combination of these. In some implementations, the substrate can include aluminum, copper, silver, gold, or platinum. In some implementations, the substrate includes gold. In some implementations, the substrate includes a silica based glass (e.g., pure silica or mixtures such as borosilicate glass). In some implementations, the substrate includes graphite, diamond, glassy carbon, or carbon nano-tubes (CNTs). In some implementations, the substrate is a chip including gold and a silica based glass.

In some implementations, the substrate includes one or more polymers. For example, in some implementations, the polymer is a natural polymer such as cellulose, natural silk, cotton, or natural rubbers. In some implementations, the polymer is a synthetic polymer, such as nylon, epoxies, polyethylene (e.g. HDPE and LDPE), polypropylene, polybutadiene, polyethylene terephthalate (PET), polycarbonate, polyurethane, fluorinated polymers (e.g. TEFLON®), polystyrene (e.g. Styrofoam), sulfonated polystyrene, aramide (e.g. KEVLAR®), poly acrylonitrile, poly vinyl acetate, poly vinyl chloride (PVC), poly methyl methacrylate (PMMA), Polyhydroxyethylmethacrylate (PolyHEMA), poly ethers, poly lactic acid, and copolymers and blends of these. In some implementations, the polymer is an ionic polymer, such as a cationic or anionic polymer.

In some implementations, the substrate or substrate surface is treated. For example, in some implementation, surfaces such as polymers (e.g. flexible substrates such as PET) may require additional modifications for the coating to adhere to the surface. For example, polyHEMA lacks functional groups and presents a challenge for adhesion. According to some implementations, one approach is to add an adherence layer such as a silane coupling layer on the polyHEMA surface which provides attachment of the protective coating through coupling chemistry. In some implementations, the substrate is functionalized with cationic functional groups such as quaternary amines. In some implementations, the substrate is functionalized with anionic groups, such as carboxylic acid groups. In some implementations, the substrate is functionalized with hydrophobic groups such as hydrocarbons. In some implementations, the substrate is functionalized with hydrophilic groups such as polyethylene oxide.

In some implementations, the surface is treated with silanes, functional groups for click chemistry, groups for avidin-biotin interaction, thiol groups, self-assembling monolayers, anionic polymerization groups, radical polymerization groups, salinization, esterification, pegylation, hydrosylation, UV treatment, ionizing radiation such as electron beam irradiation, ozone treatment, acid treatment, and plasma treatment. In some implementations, the surface is treated with a cross-linking agent, such as the cross linking agents as previously described, where the cross linking agent can cross link to the surface (e.g. through surface functional groups and one group on the linking agent) and to the protein.

The particulate material can be in any form compatible with the proteinaceous material. As use here “compatible” means the particle does not degrade/destroy the proteinaceous material and the proteinaceous material does not degrade/destroy the particulate material. In some implementations, the particulate material has a surface that can be coated with the proteinaceous material. In some implementations, the particulate material can be included in the form of a particle, a nano-particle, a micro-particle, a nano-fiber, a micro-fiber, a flake, a chip, a crystal, a porous particle, a rod, a nano-rod, a micro-rod, or combination of these forms.

In some implementations, the particulate material can be a transparent material, an opaque material, an insulator, a conductor, a semi-conductor, a dielectric material, or include a combination of these properties.

In some implementations, the particulate material includes a metal, a metalloid, a polymer, a ceramic, a glassy material, an amorphous material, a biological particle, a protein particle, a micelle, a vesicle, a cell, a carbon based material, or any combination of these. In some implementations, the particulate material can include aluminum, copper, silver, gold, or platinum. In some implementations, the substrate includes a gold. In some implementations, the particulate material includes a silica based glass (e.g., pure silica or mixtures such as borosilicate glass). In some implementations, the particulate material includes graphite, graphene oxide, reduced graphene oxide, diamond, glassy carbon, or carbon nano-tubes (CNTs).

In some implementations, the particulate material includes one or more polymers. For example, in some implementations, the polymer is a natural polymer such as cellulose, natural silk, cotton, or natural rubbers. In some implementations, the polymer is a synthetic polymer, such as nylon, epoxies, polyethylene (e.g. HDPE and LDPE), polypropylene, polybutadiene, polyethylene terephthalate (PET), polycarbonate, polyurethane, fluorinated polymers (e.g. TEFLON®), polystyrene (e.g. Styrofoam), sulfonated polystyrene, aramide (e.g. KEVLAR®), poly acrylonitrile, poly vinyl acetate, poly vinyl chloride (PVC), poly methyl methacrylate (PMMA), Polyhydroxyethylmethacrylate (PolyHEMA), poly ethers, poly lactic acid, and copolymers and blends of these. In some implementations, the polymer is an ionic polymer, such as a cationic or anionic polymer.

In some implementations, the particulate material is surface treated. For example, similarly to the substrate in some implementation, surfaces such as polymers may require modifications for the proteinaceous material to mix well with the particulate material. In some implementations, the particulate material is functionalized with cationic functional groups such as quaternary amines. In some implementations, the particulate materials are functionalized with anionic groups, such as carboxylic acid groups. In some implementations, the particulate materials are functionalized with hydrophobic groups such as hydrocarbons. In some implementations, the particulate materials are functionalized with hydrophilic groups such as polyethylene oxide.

In some implementations the coating on the substrate is composed of conducting particulate material such as a carbon allotrope (e.g., carbon nanotubes, graphene and/or reduced graphene oxide); and a denatured protein such s denatured BSA. For example, to form a denatured protein/conducting particulate material mixture that is coated on an electrode surface or other surface. In some implementations, a gold electrode that can be coated with the mixture, made with conducting particulate material and where the denatured protein is functionalized with a capture agent, such as a capture antibody. The captured antigen, such as an IL6, is detected with a biotinylated detection antibody conjugated to streptavidin-polyHRP. Upon capture of the antigen, the TMB is oxidized, and precipitates onto the electrode surface where it can be detected electrochemically (e.g., by reduction, or reduction and oxidation cycles such as used in cyclic voltammetry). In some implementations, the coating can be used to either (i) block an electrode already modified with a capture agent, or in some embodiments (ii) coat a clean electrode and later be modified with the capture agent.

As used herein, a “capture agent” or “capture molecule” is a natural or synthetic receptor (e.g., a molecular receptor) that binds to a target molecule. In some implementations, the capture agent is a “capture antibody.” In some implementations the binding is a specific binding such that it is selective to that target above non-targets. For example the dissociation constant between the capture agent and target is at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater. In certain embodiments, the specific binding refers to binding where the capture agent binds to its target without substantially binding to any other species in the sample/test solution.

By way of non-limiting examples, a capture agent can be an antibody, adnectins, ankyrins, other antibody mimetics and other protein scaffolds, aptamers, nucleic acid (e.g., an RNA or DNA aptamer), protein, peptide, binding partner, oligosaccharides, polysaccharides, lipopolysaccharides, cellular metabolites, cells, viruses, subcellular particles, haptens, pharmacologically active substances, alkaloids, steroids, vitamins, amino acids, avimers, peptidomimetics, hormone receptors, cytokine receptors, synthetic receptors, sugars or molecularly imprinted polymer. The capture agent is selective to a specific target or class of targets such as toxins and biomolecules. For example, the target can be ions, molecules, oligomers, polymers, proteins, peptides, nucleic acids, toxins, biological threat agents such as spore, viral, cellular and protein toxins, carbohydrates (e.g., mono saccharides, disaccharides, oligosaccharides, polyols, and polysaccharides) and combinations of these (e.g., copolymers including these).

In some implementations the capture agent is an antibody. As used herein, the terms “antibody” and “antibodies” include polyclonal antibodies, monoclonal antibodies, humanized or chimeric antibodies, single chain Fv antibody fragments, Fab fragments, and F(ab)₂ fragments. Antibodies having specific binding affinity for a target of interest (e.g., an antigen) can be produced through standard methods. As used herein, the terms “antibody” and “antibodies” refer to intact antibody, or a binding fragment thereof that competes with the intact antibody for specific binding and includes chimeric, humanized, fully human, and bispecific antibodies. In some embodiments, binding fragments are produced by recombinant DNA techniques. In additional embodiments, binding fragments are produced by enzymatic or chemical cleavage of intact antibodies. Binding fragments include, but are not limited to, Fab, Fab′, F(ab′)₂, Fv, and single-chain antibodies.

In some embodiments the target of the capture agent can be redox active (e.g., an electroactive capture agent) and is directly detected by an electrode. For example, the capture agent facilitates detection of the target analyte by the electrode due to it concentrating the analyte near or at the surface of the electrode where it can be detected directly by electrochemical means. In some implementations, the electrode is a gate electrode of a Field Effect Transistor (FET) and the change in concentration of the target of the capture agent changes the voltage in a range to activate/deactivate the gate.

In some other implementations the target is detected indirectly by electrochemical means. For example, the target can be detected by binding with a detection agent such as an antibody, protein or molecule that catalyzes, directly or indirectly, a redox reaction close to an electrode surface. Optionally, the detection agent, antibody, protein or molecule deposits a sacrificial redox active molecule on the electrode surface (e.g., on a coating that is on the metal surface of the electrode) that then is detected electrochemically. For example, the detection antibody can be conjugated with a redox catalyst and the sacrificial redox active molecule can be oxidized or reduced and precipitated onto the electrode surface. In some implementations, the redox active catalyst is a peroxidase such as horseradish peroxidase (HRP) and the sacrificial redox active molecule is 3,3′-Diaminobenzidine (DMB); 2,2′-azino-bis(3-ethylbenzothiazoline-6-sulphonic acid) (ABTS); o-orthophenylenediamine (OPD); AmplexRed; 3,3′-Diaminobenzidine (DAB); 4-chloro-1-naphthol (4CN); AEC; 3,3′,5,5′-Tetramethylbenzidine (TMB); homovanilllic acid; lumininol; Nitro blue tetrazolium (NBT); Hydroquinone; benzoquinone; mixtures of these; or mixtures of these. Embodiments include known immunoassays or modifications of these to be detectable by electrochemistry. Optionally, the sacrificial molecule can also be detected by fluorescence.

In some implementations, the capture agent is used at a concentration between about 10 and about 5000 μ/mL. In some implementations, the capture agent is used at a concentration between about 50 and 1000 μ/mL, such as between about 100 and 1000 μ/mL, or between about 100 and about 1000 μ/mL. In some implementations, the detection agent is (e.g., detection antibody) is used at a concentration between about 0.1 and 100 μ/mL, such as between about 0.5 and 50 μ/mL, between about 1 and 20 μ/mL, between about 1 and 8 μ/mL, or between about 2 and 5 μ/mL. In some implementations, the capture agent includes streptavidin-polyHRP or a similar molecule for signal augmentation. In some implementations, the streptavidin-polyHRP concentration is between about 0.1 and about 100 μ/mL, such as between about 0.5 and 50 μ/mL, or between about 1 and 10 μ/mL. The ranges of concentrations of capture agent and detection agent can be used in any combination, such as 500 μ/mL of capture agent in combination with 5 μ/mL of detection agent. The ranges of concentrations of capture agent, detection agent and streptavidin-polyHRP also be used in any combination, such as 500 μ/mL of capture agent, 5 μ/mL of detection agent and 2 μ/mL streptavidin-polyHRP.

As used here a “conductive surface” is an outer surface of a bulk conductive material. For example, any surface of a metal sheet, bar, wire, electrode, contact, etc. This can include porous materials, polished materials or materials with any surface roughness, surfaces that are substantially flat or have some curvature (e.g., concave or convex). Conductive surfaces include surfaces of non-metallic materials that are poor conductors or good conductors, such as, for example graphite, Indium tin oxide (ITO), semiconductors, conductive polymers and materials used for making electrodes. For example, the conductivity can be in the range between a semiconductor (e.g., about 1×10³ S/m) and a metal (e.g., about 5×10⁷ S/m). In some implementations, the conductive surface is the part of an electrode that is coated with a protective coating such as CNTs/BSA or rGO/BSA compositions, and then contact with the sample that is being probed for an electrochemical response.

In some embodiments, the coated surface provides protection and anti-fouling properties to the surface of the substrate. For example, where an electric transducer surface is protected in a complex matrix. As used here a “complex matrix” can include biomolecules, molecules, ions, cells, organisms, inorganic materials, liquids and tissue. For example, a complex matrix can include biological fluids; such as blood, serum, plasma, urine, saliva, interstitial fluid and cytosol; and tissues such as from a biopsy and tissues on a living organism (e.g., an implant, a diagnostic probe).

As used herein an “electrode” is a conductor through which current enters or leaves a medium, where the medium is nonmetallic. For example, the medium can be a complex matrix (e.g., blood or serum). The electrode can be inserted into/onto a tissue such as mammalian tissue and be contacted with tissue and/or fluids therein/thereon. The electrode can be large (e.g., with a working surface area of greater than 1 cm², greater than 10 cm², greater than 100 cm²) or the electrode can be small (e.g., with a working surface area of less than 1 cm², less than 1 mm², less than 100 μm², less than 10 μm², less than 1 μm²). The working surface area is the area in contact with the medium and wherein current enters or leaves the medium. In some embodiments the electrode is a working electrode and the electrochemical cell can include a counter electrode and reference electrode.

In some embodiments the electric transducer, such as an electrode, is “Multiplexed” such that it is configured for a multiplexed assay. As used herein a “multiplexed” assay can be used to simultaneously measure multiple analytes or signals such as two or more (e.g., 3 or more, 5 or more, 10 or more, 50 or more, 100 or more, 1000 or more) during a single run or cycle of the assay. The electric transducer can therefore be configured as an array of transducers, electrodes, microelectrodes or electrochemical sensors each of which can be independently electrically attached to a circuit for monitoring the electrical signals. For example, the array of electrodes can be disposed at the bottom, sides or top of a multiwell plate (e.g., microwell plate) arrayed on a flat surface such as a semiconductor chip (e.g., a sensor array chip) or form part of a multielectrode array (e.g., for connection of neurons to electronic circuitry). In some embodiments, the coatings can coat more than one sensor since the coating will not conduct between the sensors due to the anisotropy of the conduction, therefore an array of conductors, sensors or electrodes can be coated forming a multiplexed electrode.

Electric transducers can include materials with metallic conduction and semiconductors. For example, electric transducers can include metals, metal alloys, semiconductors, doped materials, conducting ceramics and conducting polymers. Without limitation, electric transducer materials can include carbon (e.g., graphite, glassy carbon, conductive polymers), copper, titanium, brass, mercury, silver, platinum, palladium, gold, rhodium, zinc, lead, tin, iron, Indium Tin Oxide (ITO), silicon, doped silicon, II-VI semiconductors (e.g., ZnO, ZnS, CdSe), III-V semiconductors such as (e.g. GaAs, InSb), ceramics (e.g. TiO₂, Fe₃O₄, MgCr₂O₄), and conductive polymers (e.g., poly(acetylene)s, poly(p-phenylene vinylene), poly(fluorenes)s, polyphenylenes, polypyrenes, polyazulenes, polynaphthalenes, polyanilines, polyazepines, polyindoles, polycarbazoles, poly(pyrrole)s, poly(thiophene)s, and poly(3,4-ethylenedioxythiophene)), combinations, mixtures and alloys of these.

In some implementations, the electric transducers are implemented to provide detection of an analyte using electrochemical methods. Electrochemical methods are methods that rely on a change in the potential, charge or current to characterize the analyte's chemical reactivity. Some examples include potentiometry, controlled current coulometry, controlled-potential coulometry, amperometry, stripping voltammetry, hydrodynamic voltammetry, polarography, stationary electrode voltammetry, pulsed polarography, electrochemical impedance spectroscopy and cyclic voltammetry. The signals are detected using an electrode or electrochemical sensors coupled to circuits and systems for collection, manipulation and analysis of the signals.

As used herein “carbon nanotubes” and “graphene” are allotropes of carbon with sp 2 carbon atoms arranged in a hexagonal, honeycomb lattice. Single layer graphene is a two-dimensional material, and is a single layer of graphite. As used herein, more than one layer of graphene can be referred to as graphene, for example between 1 and 200 layers (e.g., about 1 to 100 layers, about 1 to 50 layers, about 1 to 10 layers). Carbon nanotubes are hollow, cylindrical structures, formed as a sheet of graphene rolled into a cylinder. The allotropes of carbon can include some functionalization, such as oxygen, carboxylates, epoxides, amines, amides and combinations of these, as described below. In some implementations, the functionalization includes poly amine functionalization such as pentaamine functionalization.

Graphene can be produced is high purity using chemical vapor deposition on clean metal surfaces and through exfoliation of pure graphite. The exfoliation method of graphite includes using an adhesive which is pressed on the graphite surface repeatedly until a few or even one layer is obtained. These methods can be laborious and impractical, although they can produce graphene that is pure (e.g., greater than 99 wt. % carbon). As will be described below, reduced graphene oxide (rGO) can be used in many applications where graphene is useful since it has similar electrical, chemical and mechanical properties. Reduced graphene also has some advantages, such as chemically reactive oxygen based groups that can be exploited for further chemical transformations. In addition, rGO can be prepared more efficiently. In any case, both pure graphene and reduced graphene oxide can be used in embodiments for making non-fouling coatings.

An efficient process for forming graphene oxide is the exfoliation of graphite oxide. As used herein “graphene oxide” is a material that can be formed from the oxidation of graphene or exfoliation of graphite oxide. In a first step for producing graphene oxide, graphite is oxidized. Several methods for oxidation are known, one common method known as the Hummers and Offeman method, in which graphite is treated with a mixture of sulphuric acid, sodium nitrate and potassium permanganate (a very strong oxidizer). Other methods are known to be more efficient, reaching levels of 70% oxidisation, by using increased quantities of potassium permanganate, and adding phosphoric acid combined with the sulphuric acid, instead of adding sodium nitrate. Exfoliation of graphene oxide provides graphite oxide and can be done by several methods. Sonication can be a very time-efficient way of exfoliating graphite oxide, and it is extremely successful at exfoliating graphene (almost to levels of full exfoliation), but it can also heavily damage the graphene flakes, reducing them in surface size from microns to nanometres, and also produces a wide variety of graphene platelet sizes. Mechanically stirring is a much less destructive approach, but can take much longer to accomplish.

Graphite oxide and graphene oxide are very similar, chemically, but structurally, they are very different. Both are compounds having carbon, oxygen and hydrogen in variable ratios. In the most oxidized state the oxygen amount can be as high as about 60 wt %. the amount of hydrogen varies depending on the functionalization, for example, the number of epoxy bridges, hydroxyl groups and carboxyl groups. The main difference between graphite oxide and graphene oxide is the interplanar spacing between the individual atomic layers of the compounds, caused by water intercalation. This increased spacing, caused by the oxidisation process, also disrupts the sp 2 bonding network, meaning that both graphite oxide and graphene oxide are often described as electrical insulators.

Reduced graphene oxide (rGO) is prepared from reduction of graphene oxide by thermal, chemical or electrical treatments. For example, treating the graphene oxide with; hydrazine, hydrogen plasma, heating in water, high temperature heating (e.g., under nitrogen/argon) and electrochemical reduction. Whereas graphene can be a single carbon layer ideally comprising only carbon, reduced graphene oxide is similar but contains some degree of oxygen functionalization. The amount of oxygen depends on the degree of reduction and in some materials can vary between about 50 wt % and about 1 wt. % (e.g., between about 30 wt. % and about 5 wt. %).

Reduced graphene oxide can be functionalized or include functional groups. For example, reduced graphene oxide often includes oxygen in the form of carboxyl groups and hydroxyl groups. In some forms, the carboxyl and hydroxyl groups populate the edges of the rGO sheets. As used herein, carbonylated reduced graphene oxide can refer to reduced graphene oxide having carboxyl groups. In some embodiments the amount of oxygen attributable to the carboxyl groups is between about 30 wt. % and about 0.1 wt. % (e.g., between about 10 wt. % and about 1 wt. %). Other forms of functionalization are possible. For example, amine functionalized rGO can be formed by a modified Buchere reaction, wherein ammonia an graphene oxide are reacted using a catalyst such as sodium bisulfite, or epoxide groups on graphene oxide can be opened with p-phenylenediamine. In some embodiments, the amount of nitrogen is between about 30 wt. % and 0.1 wt. % (e.g., between about 10 wt. % and 1 wt. %). In some implementations, a polyamine is used to functionalize rGO. For example, pentaamine functionalized graphene is used in some implementations.

The tube-shaped carbon nanotubes have diameters in the nanometer scale, such as, for example, between about 0.2 and about 20 nm, preferably between about 0.5 and about 10 nm, and more preferably still between about 1 and about 5 nm. These can be single walled carbon nanotubes (SWCNT), multi walled carbon nanotubes (MWCNT) (e.g., a collection of 2 or more nested tubes of continuously increasing diameters, or mixtures of these). The diameters of MWCNT can be larger than the SWCNT, such as between about 1 and about 100 nm (e.g., between about 1 and about 50 nm, between about 10 and 20 nm, between 5 and 15 nm, between about 30 and 50 nm). Depending on how the precursor graphene sheet is rolled up to make a seamless cylinder that is the carbon nanotube, different isomers of carbon nanotube can be made, for example designated as armchair configuration, chiral configuration, and zigzag configuration.

The carbon nanotubes and reduced graphene oxide can include intercalated materials, such as ions and molecules. In some embodiments the carbon nanotubes can be functionalized for example by oxidation to form carboxylic acid groups on the surface, providing CNTs. In addition, in some embodiments, the carbon nanotubes and rGO can be further modified through condensation reactions with the carboxylic acid groups present on the CNTs or rGO (e.g., with alcohols and amines), electrostatic interactions with the carboxylic acid groups (e.g., calcium mediated coupling, or quaternary amines, protonated amine-carboxylate interaction, through cationic polymers or surfactants) or hydrogen bonding through the carboxylic acid groups (e.g., with fatty acids, and other hydrogen bonding molecules). The functionalization can be partial (e.g., wherein less than 90%, less than 80%, less than 60%, less than 50%, less than 40%, less than 30%, less than 20%, less than 10%, more than 10%, more than 20%, more than 30%, more than 40%, more than 50%, more than 60%, more than 70%, more than 80%, of the available carboxylic acid groups are functionalized) or complete, such as functionalizing substantially all the carboxylic acids (e.g., more than 90%, more than 95%, more than 99% of available carboxylic acid groups). In some embodiments the functionalization can be with a redox active compound or fragment (e.g., a metallocene, a viologen), antibody, a DNA strand, an RNA strand, a peptide, an antibody, an enzyme, a molecular receptor, a fragment of one of these or combination of these.

The allotropes of carbon having hexagonal lattices of carbon atoms, such as CNTs and rGO, can confer electroactivity (e.g., conductivity) to the compositions and structures herein described. Other conductive elements such as pure graphene, fullerenes, conductive and semi-conductive particles, rods, nano-rods, micro-rods, fibers, nano-fibers, micro-fibers, particles, nanoparticles and micro-particles (e.g., Gold), and conductive polymers (e.g., polypyrrole, polythiophene, polyaniline) can also be used to replace the CNTs and rGO or blended/combined with CNTs to modulate (e.g., improve) the conductivity, improve the stability and/or improve the stability of the coatings.

Interestingly, some of the embodiments described herein show anisotropy in conductivity. In some embodiments the coatings conduct in a direction perpendicular to the surface of an electrode, equivalent herein to “vertically”, to a greater degree than in directions parallel or tangential to the surface of the electrode, equivalent herein to “laterally”. In Cartesian coordinates this can correspond to higher conduction in the z direction (perpendicular to the electrode surface) than in the x and y directions (e.g., combinations of x and y pointing vectors). For example, the conductivity in the vertical direction is at least two times (e.g. at least 3 times, 4 times, 5 times, 10 times, 100 times, 1000 times) higher than that in the lateral direction.

The mixture of particulate material and proteinaceous material can be coated by any method. As non-limiting examples, these methods include spraying, spin coating, dip coating, inkjet printing, vapor deposition, 3-D printing, painting, and/or drop casting. In some implementations, the methods include continuous or semi-continuous methods. For example, the mixture can be spin or dip coated on a wafer having a plurality of electronic transducers patterned thereupon. Each spin or dip coating step can be part of a multi-step process in a fab or foundry (e.g. a semiconductor fabrication plant) involving a continuous train of wafers. Such a process can be considered as semi-continuous. Alternatively, coating can be using a reel to reel (or roll to roll) method. Reel to reel processing is a fabrication method used in manufacturing that embeds, coats, prints, or sprays the mixture onto a flexible rolled substrate material, such as a web or sheet, as that material is fed continuously from one roller on to another roller. Reel to reel is a continuous process.

In some implementations, the particulate material and proteinaceous material is coated immediately before it is used. For example, an electrode or other device is coated and immediately and used in an assay within an hour of preparing the coating. However, in some implementations, the particulate material and proteinaceous material forms a very robust coating and can be used at least a day, at least two days, at least a week, at least two weeks, at least a month, at least three months, at least six months, at least a year, at least two years after preparation, where the electrode or device signal retains at least about 90% of its original signal. In some implementations, the electrode or device that is coated with the particulate material and proteinaceous material is stored between about 0° C. and 50° C. before being used, for example between about 0° C. and 50° C., between about 0° C. and 40° C., between about 10° C. and 50° C., between about 20° C. and 40° C., between about 20° C. and 30° C.

In some implementations, an electrode or device with the particulate material and proteinaceous material coating can be used in an assay such as a precipitated sacrificial redox active molecule on the electrode surface (e.g., containing TMB), where the signal can be detected at least a day, at least two days, at least a week, at least two weeks, at least a month, at least three months, at least six months, at least a year, at least two years after the precipitation of the redox active material. In some implementations, the electrode or device that is coated with the particulate material and proteinaceous material, and includes a sacrificial redox active molecule, is stored between about 0° C. and 50° C. before the redox active molecule is detected, for example between about 0° C. and 50° C., between about 0° C. and 40° C., between about 10° C. and 50° C., between about 20° C. and 40° C., between about 20° C. and 30° C.

In some implementations, the methods can be used to make multilayer materials, alternating between a layer of the coating of proteinaceous material and a substrate material. For example; a first mixture comprising a first particulate material and a first proteinaceous material is coated on a first substrate, providing a first proteinaceous layer on the first substrate; a second mixture comprising a second particulate material and a second proteinaceous material is coated on a second substrate, providing a second proteinaceous layer on the second substrate; a third mixture comprising a third particulate material and a third proteinaceous material is coated on a third substrate, providing a third proteinaceous layer on the third substrate; etc. In some embodiments more than 3 layer alternating between the substrate layer and the proteinaceous layer (e.g. more than 4, 5, 6, 10, 20, 50 layers). In some implementations, the layered material can start with a substrate material, with the final layer being a substrate material, or with the final layer being a substrate material. In some implementations, the layered material can start with a substrate material, with the final layer being a substrate material, or with the final layer being a substrate material. Each of the compositions of particulate materials and proteinaceous materials for the coatings (e.g. first, second, third) can be independently selected in composition and manner of coating. Each of the substrate materials for the coatings (e.g. first, second, third) can be independently selected in composition and manner of coating. Such multilayer films can be prepared, for example, by standard semiconductor manufacturing methods. For example, in some implementations, substrates such as metals can be layered onto the proteinaceous coatings by sputtering, vapor phase deposition, electrodeposition etc. In some implementations, the mixtures of particulate material and proteinaceous material are sprayed or spin coated on the substrate layers. In some implementations, patterning, creation of vias (e.g. conductive vias such as copper or aluminum), lines (e.g., conductive lines such as copper or aluminum) by combinations of deposition, etching, masking and planarization can also be implemented. In some implementations, the pattering includes through holes providing access to the internal layers in the internal structure such as can be accessed by analytes.

According to some implementations, the anti-fouling coatings described herein can be applied to surfaces of microfluidic devices. These devices can include channels through which fluids can flow and chambers for holding the fluids. Electronic transducers can be included and the anti-fouling coatings can be used to protect these and other parts of the device. In some implementations, the coating is a cross-linked and porous gel, and a channel or chamber is completely or mostly filled in. Analyte can flow through the pores and be detected, for example, by binding to a capture molecule such as an antibody, DNA strand, or aptamer. In some implementations, the coatings can be patterned to provide hydrophilic/hydrophobic areas for movement or holding of fluids. In some implementations, the coatings can be patterned as a conductive wire or a dielectric/insulating surface. Some implementations include coating microfluidic chips, lab-on-a-chip, and organs on a chip.

In some implementations, the coatings can be used in nano-gap and micro-gap devices. For example, these devices include nano-gap electrodes, nanostructured-based electrical biosensors, and nano-gap dielectric biosensor for label free DNA hybridization detection. The coatings can be applied, for example, to the gap between electrodes in the device and thereby protect the surfaces of the gap from fouling. In some implementations, the coating is a cross-linked and porous gel, and the gap is completely or mostly filled in. Analyte can flow through the pores and be detected, for example, by binding to a capture molecule such as an antibody, DNA strand, or aptamer.

In some implementations, the coatings are used for medical devices. For example, the tips of needles can be coated. In some implementations, the medical device is an implantable device. For example, continuous glucose monitors, pace makers, implantable cardioverter defibrillators, blood filers, cochlear implants, and blood monitors. In some implementations, the coatings can be used in lateral flow assay devices. In some implementations, the coatings can be used for wearable devices, such as transdermal patches, devices to detect biological agents or toxins.

Some exemplary embodiments of the disclosure can be described by the following numbered embodiment:

Embodiment 1: A method for making a coating on a surface of a substrate, the method comprising: applying a mixture to a surface of a substrate while maintaining the substrate at an elevated temperature, wherein the mixture comprises a particulate material and a proteinaceous material.

Embodiment 2: The method of Embodiment 1, wherein the mixture further comprises a cross-linking agent.

Embodiment 3: The method of Embodiment 1 or 2, wherein the proteinaceous material includes a cross linking agent attached to or as part of the proteinaceous material's structure.

Embodiment 4: The method of any one of Embodiments 1-3, wherein the elevated temperature is maintained for at least 10 seconds and less than two minutes.

Embodiment 5: The method of any one of Embodiments 1-4, wherein the elevated temperature is at least 50° C.

Embodiment 6: The method of any one of Embodiments 1-5, wherein the method further comprises denaturing the proteinaceous material.

Embodiment 7: The method of Embodiment 6, wherein said denaturing the proteinaceous material is prior to mixing the proteinaceous material with the particulate material and/or after applying the mixture to the substrate.

Embodiment 8: The method of any one of Embodiments 1-7, wherein the substrate is a particle, a nano-particle, a micro-particle, a nano-fiber, a micro-fiber, a flake, a chip, a crystal, a porous substrate, a wafer, a wire, a nano-wire, a micro-wire, a channel, a nano-channel, a micro-channel, a rod, a nano-rod, a micro-rod, a foil, a sheet, a web, or combination of these forms.

Embodiment 9: The method of any one of Embodiments 1-8, wherein the substrate comprise a material selected from the group consisting of metals, polymers, carbon based materials, ceramics, glass and any combinations thereof.

Embodiment 10: The method of any one of Embodiments 1-9, wherein the substrate includes gold.

Embodiment 11: The method of any one of Embodiments 1-10, wherein the substrate includes graphite, diamond, glassy carbon, or carbon nano-tubes.

Embodiment 12: The method of one of Embodiments 1-11, wherein the substrate includes an organic polymer.

Embodiment 13: The method of any one of Embodiments 1-12, wherein the particulate material is a rod, fiber, a particle, a flake or combinations of these.

Embodiment 14: The method of any one of Embodiments 1-13, wherein the particulate material is a dielectric.

Embodiment 15: The method of any one of Embodiments 1-14, wherein the particulate material is a conductor or semi-conductor.

Embodiment 16: The method of any one of Embodiments 1-15, wherein the particulate material comprises an allotrope of carbon atoms arranged in a hexagonal lattice.

Embodiment 17: The method of any one of Embodiments 1-16, further comprising a step of pre-treating the substrate prior to applying the mixture.

Embodiment 18: The method of any one Embodiments 1-17, wherein applying the mixture comprises spraying, spin coating, dip coating, inkjet printing, vapor deposition, 3-D printing, painting, and/or drop casting.

Embodiment 19: The method of any one of Embodiments 1-18, wherein the method is a continuous process or semi-continuous process (e.g. reel to reel, pattern deposition on a wafer).

Embodiment 20: The method according to any one of Embodiments 1-19, further comprises denaturing the proteinaceous material and subsequently adding a temperature sensitive material to the mixture prior to coating the substrate.

Embodiment 21: The method according to any one of Embodiments 1-20, wherein the substrate surface defines a channel or chamber, such as a channel or chamber in a microfluidic device.

Embodiment 22: The method according to any one of Embodiments 1-21, wherein the substrate is a micro/nano gap devices where the coating can enable higher sensitivity and the coating can either be used to coat the surfaces of a gap in the micro/nano gap device, or the coating is applied for surface modification to enable linking of specific probes and antifouling properties.

Embodiment 23: The method of any one of Embodiments 1-22, further comprising applying a layer of a second substrate on the coating of proteinaceous material and optionally coating a second mixture comprising a second mixture and second proteinaceous material on the second substrate, providing a layered material having alternating layers of substrate and proteinaceous/particulate material.

Embodiment 24: A substrate comprising a coating on a surface thereof, wherein said coating is applied using a method of any one of Embodiments 1-23.

Embodiment 25: The substrate of Embodiment 24, wherein the substrate is an electrode, a capacitor, a bio-Field Effect transistor (bio-FET), transistors, and optical devices.

Embodiment 26: A capacitor comprising a dielectric material dispersed in a denatured and cross-linked proteinaceous material, and covering a conductive substrate.

Embodiment 27: A bio-FET comprising a composition including a particulate material dispersed in a denatured proteinaceous material and coating at least a portion of a transistor.

Embodiment 28: The bio-FET according to Embodiment 27, wherein the compositing is coated on a gate of the bio-FET.

The embodiments will be more readily understood by reference to the following examples, which are included merely for purposes of illustration of certain aspects and embodiments of the present invention, and should not be construed as limiting. As such, it will be readily apparent that any of the disclosed specific constructs and experimental plan can be substituted within the scope of the present disclosure.

### Examples

Current electrochemical coating methods suffer either from long and intricate fabrication techniques or lacking effective anti-fouling properties, hindering the sensors from detecting extremely low levels of biomarkers and limiting their mass manufacturing capabilities. Herein is described a robust, ultra-fast anti-fouling nanocomposite coating method for electrochemical (EC) sensors accomplished within 1 minute, which is an amalgamate of denatured bovine serum albumin and pentaamine modified graphene oxide nanoparticles with glutaraldehyde. The fine-tuned, cost-effective one-pot recipe can be stored at room temperature for months allowing for mass manufacturing capabilities with reduced complexity. The unprecedented selectivity of the coating allowed the development of a multiplexed platform that can be used to triage patients suffering from Myocardial Infarction and Traumatic Brain Injury using only 15 μL of the sample. A single-digit pg/mL sensitivity was obtained with all the markers tested in unprocessed human plasma samples and whole blood, which is at least 50 times more sensitive than traditional ELISA methods and accomplished in one-eighth of the time. Furthermore, the signal adsorbed on the coating could be conserved and measured over one week. The platform was validated with 22 clinical samples that demonstrated excellent correlation with obtained reported values.

One of the key aspects of developing a reliable electrochemical (EC) biosensor is establishing efficient anti-fouling surface chemistry. Many of these fouling agents are biological molecules, including cells, proteins, and neurotransmitters that are present in extremely high quantities in biological samples, including plasma, serum, and urine. These fouling agents result in background noise and a decrease of both the electrochemical signal and specificity of the EC-Biosensor limiting their commercial success. Accordingly, anti-fouling chemistry have gathered much attention recently with methods including the use of carbon materials, metallic nanoparticles, nanoporous electrodes, and (bio)materials like polymers and hydrogels. They can be applied via multiple coating methods, including physical adsorption, PEG Grafting, zwitterionic peptide self-assembled monolayer, electrochemical copolymerization, drop-casting, and magnetic core-shell polymerization but none of them are fast enough to enable robust and cost-effective mass manufacturing capabilities. In addition, they have complicated, time-consuming, and multi-step synthesis procedures, and some of them have reduced sensitivity due to poor conductivity. Therefore, depositing stable and reliable antifouling coatings directly through simple, scalable, and economical methods is extremely important to pave the way for more robust EC-Bioensors needed in point-of-care (POC) diagnostics.

A previously reported anti-fouling coating method based on gold nanowires and graphene addresses some of these limitations, but can take 24 hours to coat. Here, is reported a new coating method via localized heating, which can be completed in unprecedented timing of less than a minute and allows outstanding electrochemical signal transduction between the gold electrode and the nanocomposite surface. This was accomplished by incorporating pentaamine graphene flakes with denatured bovine serum albumin via glutaraldehyde to improve the electrochemical signal and sensitivity of the sensor. To assess the stability and reliability of the sensors, the stability of coating at room temperature was assessed over months. Furthermore, the stability of signal of adsorbed TMB was recorded over a week, opening the possibility of shipping without compromising biomarker stability.

Detecting a single biomarker is insufficient for accurate and timely diagnosis of many diseases such as Traumatic Brain Injury (TBI) and cardiovascular diseases. Cardiovascular diseases lead to 31.5% of deaths with myocardial infarction (MI), resulting in 7.4 million deaths per year. Likewise, TBI is the worldwide leading cause of mortality and morbidity in young adults, and in the U.S., roughly 1.7 million suffer from TBI each year. At least 5.3 million live with severe disabilities related to TBI. Cardiovascular consequences resulting from moderate and severe TBI has long been recognized and are associated with an increase in-hospital mortality, so timely recognition and management of these patients is required. TBI leads to heterogeneous pathology that affects multiple cells and tissue types, necessitating simultaneous measurement of multiple biomarkers similar to AMI, which require multiple biomarkers in clinical practice. Multiplexed immunoassays are required for patient stratification and monitoring of multifactorial diseases like MI and TBI. However, to date, relatively scarce protein multiplexing immunoassays have been validated for POC settings. The direct multiplexed determination of biomarkers in complex biological media remains a significant challenge due to biofouling and the lack of high-quality antibody pair resulting in cross-reactivity and preventing the scale up. The herein described ultrafast coating method addressed these challenges. The method has exceptional anti-fouling properties and limitless manufacturing capabilities in a cost-effective way, for a multiplexed platform to triage patients suffering with MI from TBI in 37 min from 15 uL of unprocessed human plasma samples.

The ultrafast coating process improves on a previous overnight treatment. The process includes heat treatment of the coating solution directly onto the biosensor surface, which leads to rapid binding of the coating solution. The physical and/or chemical structure leading to antifouling activity or maintaining physio-chemical properties (conductivity) is comparable to the overnight processes. Unlike the overnight incubation, the rapid coating method can be used for mass manufacturing for Biosensors for instance in reel to reel manufacturing of sensors and can be used, for single or multiplexed assays. In addition, the coating solution can also be applied on relatively inert materials including but not limited to plastics and polymers (Poly (methyl methacrylate), polyethylene, polystyrene) after surface modifications/treatment like plasma treatment or UV cross linking to develop anti-biofouling biosensors.

### Initial Development of Antifouling Coating

A mixture was prepared by adding 2 mg of pentaamine functionalized graphene oxide to 5 mg/mL BSA in PBS. Optionally, a recombinant BSA and other proteins can be used. The mixture was sonicated for 1 hour with 1 second on/off pulses and then heated for 5 minutes at 105.5 C. Once cooled to room temperature, the mixture was centrifuged at 16.1 rfc for 15 minutes and the supernatant was collected and used as the coating solution.

Gold on glass chips were cleaned by sonicating in acetone for 15 minutes, rinsing with IPA, and sonicating in IPA for 15 minutes. The chips were then dried with pressurized nitrogen and plasma treated. The chips were separated into two groups: “overnight” coating and “fast” 1 min coating. The overnight chips were coated with 69 μL of a mixture of 1:69 glutaraldehyde in the graphene oxide coating and incubated overnight in the dark at room temperature. The fast coating chips were first heated to 90° C. on a hot plate. A solution of 69 μL of a mixture of 2:69 glutaraldehyde in the graphene oxide containing coating solution was added to the chips. The chips were removed from heat after 1 minute.

To test the maintenance of conductivity and anti-fouling properties of the BSA-graphene oxide coating on gold electrodes, three separate measurements were taken using cyclic voltammetry: after ethanolamine (a quenching step to halt the glutaraldehyde crosslinking reaction); after 1-hour incubation of 2.5% BSA; and after overnight incubation of 2.5% BSA. Following the BSA steps, if the coating exhibits a constant signal, this shows that the method was able to create an anti-fouling environment while also preserving the conductivity of the electrodes.

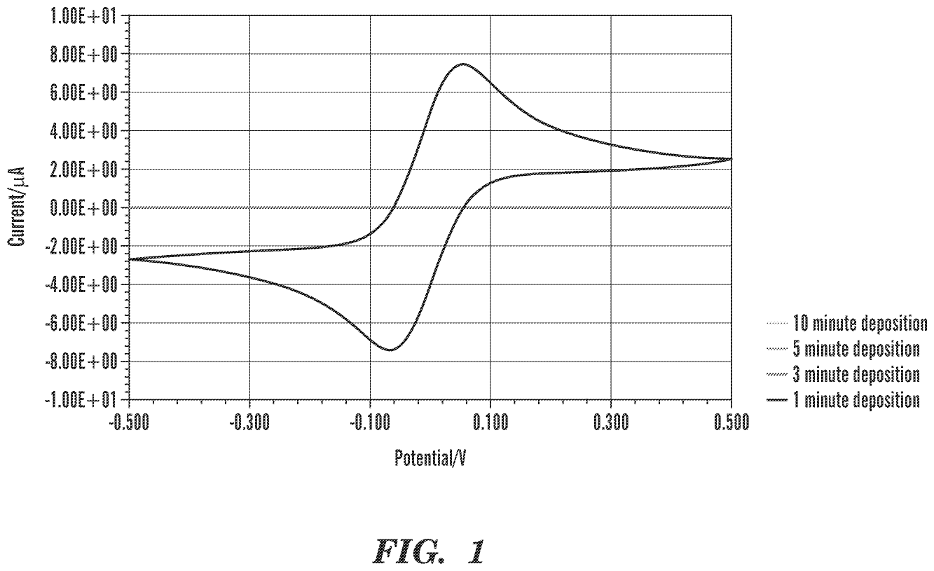

As illustrated by FIG. 1, to measure the signal of the assay, cyclic voltammetry (CV) was performed on the chips from −0.5 to 0.5 V at the scan rate of 0.2 V/s and the height of the oxidation peak current were recorded. The length of incubation on the hot plate was optimized by testing different times from 1 minute up to 10 minutes. However, no incubation period exceeding 1 minute yielded viable chips. This is shown by the CV plots where at 1 minute strong anodic/cathodic peaks are seen (about ±7.8 μA). At 3, 5 and 10 minutes no signal is observed, as shown by the overlapping flat line.

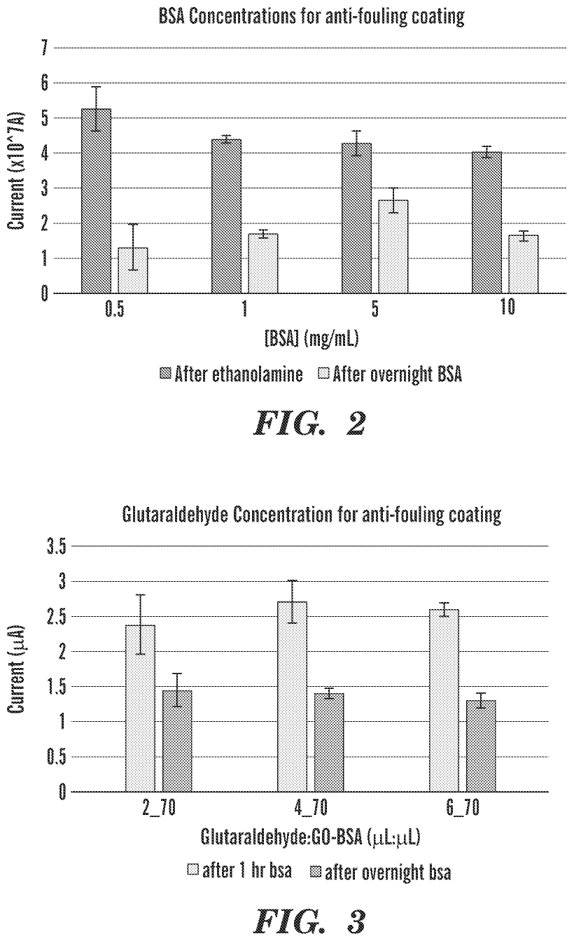

The concentrations of BSA in PBS was determined by varying the concentration from 0.5 to 10 mg/mL, as illustrated by FIG. 2. The legend represents the ratio of glutaraldehyde to graphene oxide-BSA solution. The best anti-fouling concentration was 5 mg/mL, which shows the smallest difference between the overnight coating and ethanolamine quenched samples, while having a high conductivity.

The glutaraldehyde concentration in graphene oxide—BSA mixture was varied from 1:70 (μL: μL) to 6:70. Three ratios are illustrated in the bar plot shown in FIG. 3. The recorded currents were all about equal and within the error of the measurements between the 1 hr treatments and overnight treatments. The 1:70 ratios did not yield any signal for. The best ratio was selected as 2:70, since there was no detectable difference across conditions and the least amount of glutaraldehyde was used.

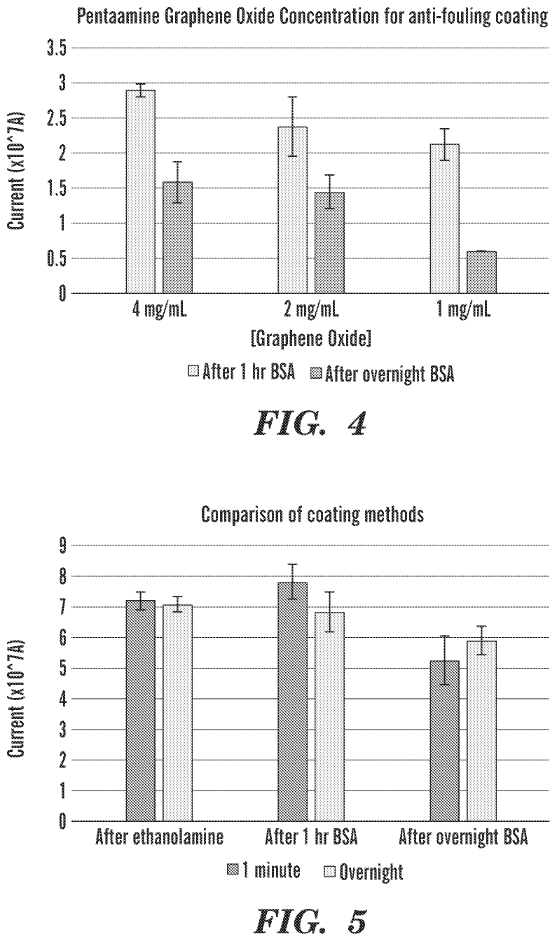

The pentaamine-functionalized graphene oxide was varied in concentration from to 4 mg/mL. FIG. 4 shows a plot of the results for three concentrations. At the dilute end of the spectrum, little to no signal was displayed. At 1 mg/mL a good signal was seen at 1 hour, which decreased overnight. At 2 mg/mL and greater the peak height reached a maximum, where the signal degradation stabilized. For example, compare 2 mg/mL and 4 mg/mL which show similar performance. Thus, the concentration of 2 mg/mL was chosen as the best concentration for optimal signal and lowest amount of graphene oxide.

In preparing the fast coating chips, the chips were allowed to cool down to room temperature and then washed with PBS for 10 minutes under in a shaker at 400 rpm. The overnight chips were similarly washed after the overnight incubation.

To verify that the fast coating method was comparable to the overnight deposition method, the same anti-fouling test was performed on two groups of chips prepared with both protocols. Measurements were taken after the ethanolamine quenching step 1 hour and overnight incubation with 2.5% BSA. FIG. 5 shows the plotted results. The 1-minute deposition method demonstrated comparably anti-fouling capabilities to the overnight method. The decrease in signal is negligible after 1 hour of BSA and about a 20-25% decrease after the overnight BSA step. This exhibits the ability of the fast coating to perform at the same level as the overnight protocol.

Following the washing of the coating, each group of chips were incubated in EDC/NHS (77 mg/23 mg) in IVIES buffer (1 mL) for 30 minutes. Chips were then rinsed with water, air dried, and the electrodes were spotted with 1 mg/mL NT-proBNP capture antibody in PBS. One electrode per chip was spotted with 5 mg/mL BSA in PBS as a negative control. Once spotted, chips were stored overnight in 4° C.

An assay for NT-proBNP was performed to determine if the fast-coating chips could perform comparably to the standard of chips with an overnight coating incubation. The morning after spotting, the electrodes were incubated in ethanolamine for 30 minutes to quench the EDC/NHS reaction. After being washed in phosphate-buffered saline with TWEEN® (PBST), the chips were then incubated in 2.5% BSA to block the electrode surface from any non-specific binding. Another wash with PBST and the varying concentrations of NT-proBNP in plasma were added: 0 ng/mL (pure filtered plasma) and 1 ng/mL. This step required 30 minutes on a shaker at 400 rpm. Following this step, the detection antibody (15 minutes), poly-streptavidin-HRP (5 minutes), and TMB One Component (1 min) were added. Washing steps with PBST were completed in between each component. To measure the signal of the assay, cyclic voltammetry was performed on the chips from −0.5 to 0.5 V at the scan rate of 1 V/s and the height of the oxidation peak current were recorded.

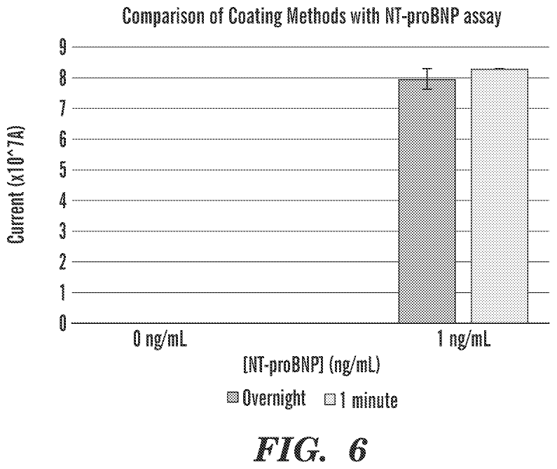

FIG. 6 illustrates the comparison of the fast and overnight protocol for deposition of an anti-fouling coating. This demonstrates that a rapid deposition coating performs equally well to the overnight protocol. Electrodes spotted with BSA had no signal for both methods, nor did electrodes that were exposed to 0 ng/mL NT-proBNP in plasma. In the absence of analyte, both methods performed appropriately by displaying a lack of signal and no non-specific binding.

The method developed is a route to implementation of fast manufacturing such as reel to reel method for mass production of sensors. Furthermore, the methods enable deposition of nano-composite layer using techniques such as spray painting over a range of surfaces including metals and polymers.

### Further Developments to Ultrafast Deposition of Antifouling Coatings

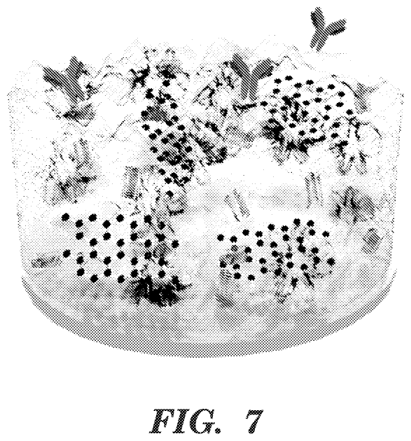

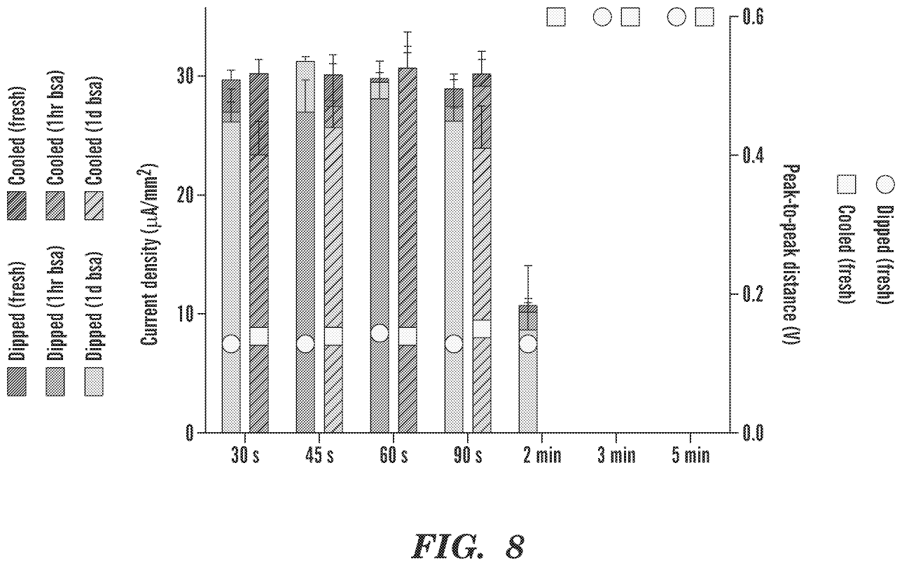

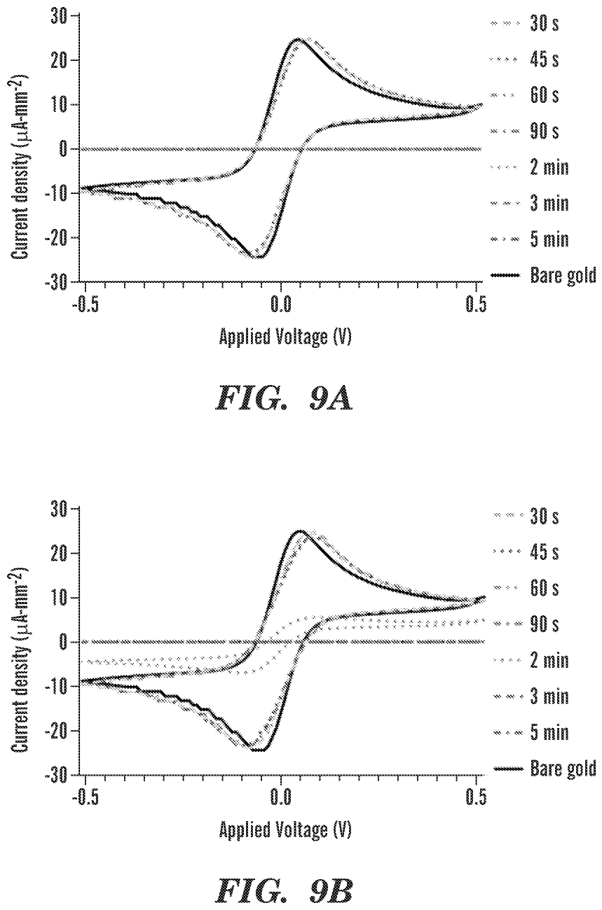

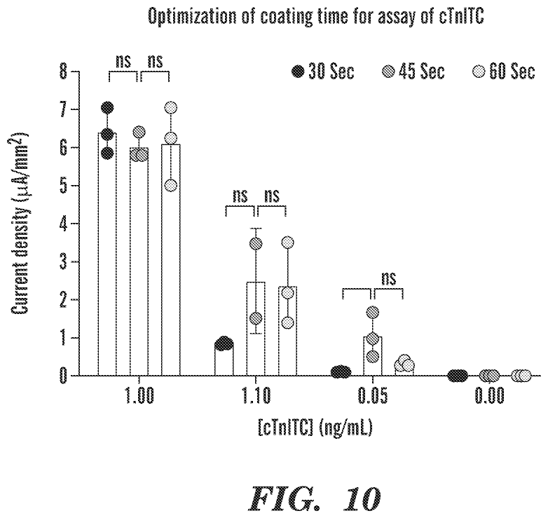

A 3D schematic of a gold electrode with antifouling nanocomposite is illustrated by FIG. 7. To develop an efficient process flow for mass manufacturing, the nanocomposite coating recipe was developed using on-chip heating to facilitate the coating deposition. Clean sensors comprising of gold electrodes were heated at 85° C. with nanocomposite material for a period ranging from 30 s to 5 min, followed by washing in PBS immediately or after cooling down for 10 min. FIG. 8 is a plot showing electrochemical characterization of the coating for the development of the optimum heating time where sensors are washed just after heating or after cooling down for 10 min. Bar shows the current density of the sensors. Cyclic voltammetry (CV) was used to evaluate the performance of different conditions of optimization steps by monitoring the oxidation and reduction peak along with the peak-to-peak distance of potassium ferri/ferrocyanide. A high current density with coated sensors up to 90 s was observed, followed by a sharp decrease in conductance. FIGS. 9A and 9B are CV plots showing typical oxidation and reduction peaks of an equimolar solution of 5 mM ferri-/ferrocyanide of sensor coated with nanocomposite at 85° C. for different periods followed by immediate dipping in water at room temperature (9A) and cooling to room temperature before dipping (9B. The sharp decrease post 90 s could be attributed to over crosslinking of the nanocomposite on the sensor surface, leading to passivation of the surface. The CV obtained using the coating shows a near reversible process with a peak-to-peak distance of 143 mV. Thereafter, the sensors coated for 30, 45, and 60 s were functionalized with antibodies, and a simple EC-Assay of cTnITC was performed in parallel. Although there was no significant difference in current density at higher concentrations of cTnITC (1 and 0.1 ng/mL) at lower concentration (0.05 ng/mL), EC-Biosensor coated for 45 and 60 s gave higher current compared to other EC-Biosensors. FIG. 10 is a plot showing optimization of coating time for sensors. Bar graph shows the assay of cTnITC with sensors coated with antifouling coating for 30 s (black dot), 45 s (blue dot), and 60 s (red dot). In addition, coating for 45 s also provides more room for coating time variation; thus, 45 s of coating time was used for further studies and characterization.

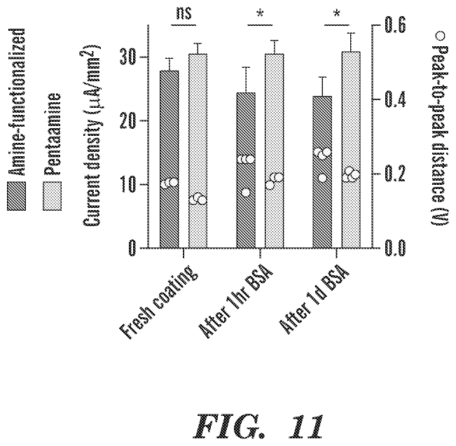

Altering the terminal groups of reduced graphene nanoparticles (rGOx) by incorporating functional groups like amines augments the interaction with the polymer matrices and distribution of the nanoflakes by altering its solubility and agglomeration and also enhance the physical, mechanical, thermal, and electrical stability through the covalent linking by glutaraldehyde pyridine polymers. To explore this further, the previously used amine-functionalized rGOx was compared to pentaamine-functionalized rGOx (prGOx). prGOx maintained a higher current density and lower peak-to-peak distance than amine-functionalized rGOx in fresh coating and coating after incubating in 1% BSA for 1 hour and 1-day, showing a significant increase in antifouling activity for prGOx. FIG. 11 is a plot showing optimization of rGOx. Bars are the current density of amine-functionalized GOx (black) and pentaamine functionalized GOx (grey).

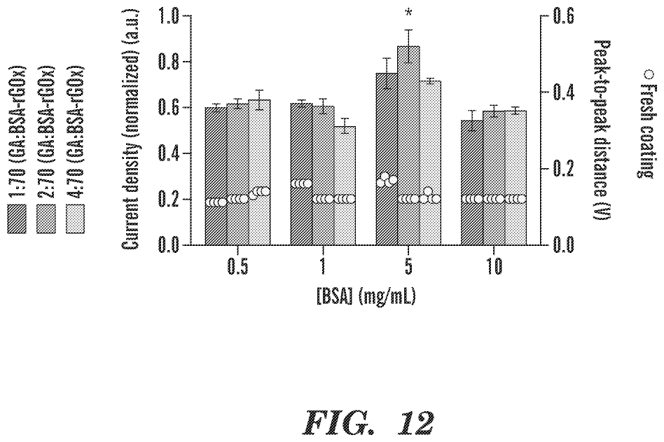

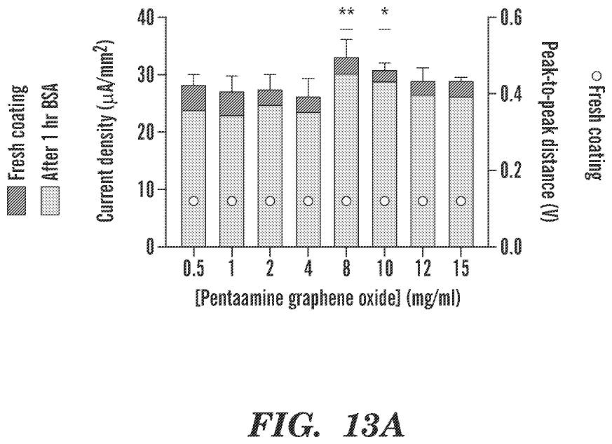

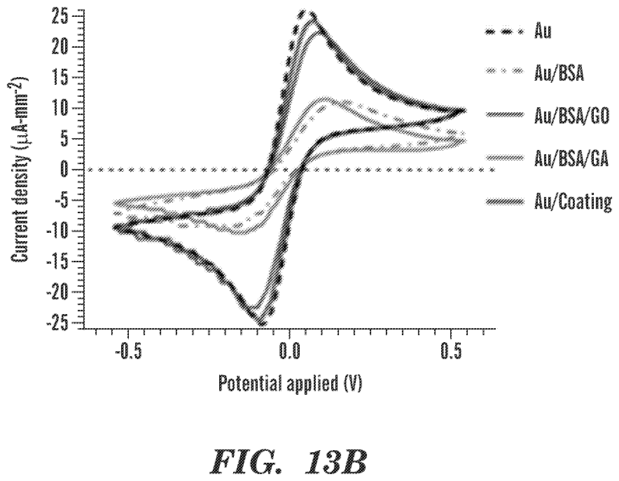

Each component of the coating was further optimized for enhanced anti-fouling electrochemical performance. FIG. 12 shows that with the increase in the concentration of BSA from 0.5 to 5 mg/mL (with all ratios of GA), the current density of the sensor increases. However, further increase (to 10 mg/mL) leads to a decrease in current density, although peak-to-peak distance remains consistent. The insulation effect at higher concentration could be due to increased cross-linking as GA can react with proteins through various mechanisms depending upon different forms it can be present in solution, which is primarily developed through empirical observation. 5 mg/mL of BSA with 2:70 ratio of GA:BSA was used for further characterization as it maintained the highest conductance. To further optimize the amalgamate of BSA/prGOx/GA, different concentrations of prGOX (0.5-15 mg/mL) were prepared in 5 mg/mL BSA and mixed with glutaraldehyde at a 2:70 ratio. As seen in FIG. 13A, highest conductivity of the nanocomposite was observed at 8 mg/mL and decreased with further increase in the concentration of prGOx. At 8 mg/mL of prGOx, there is no significant decrease in current even after 1 hr incubation in 1% BSA, which shows the excellent anti-biofouling activity of the nanocomposite. In the plot shown by FIG. 12, bars are the normalized values of the current density of nanocomposite with different concentrations of BSA at different ratios of GA to BSA/prGOx;

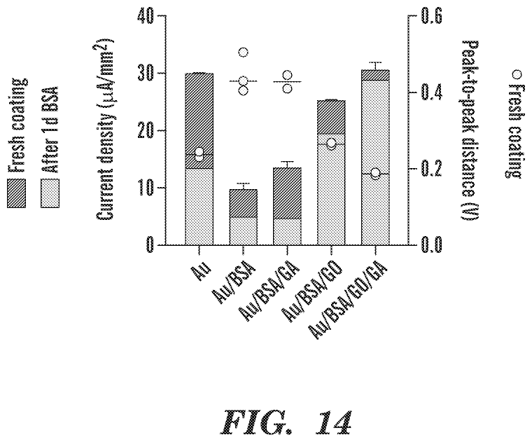

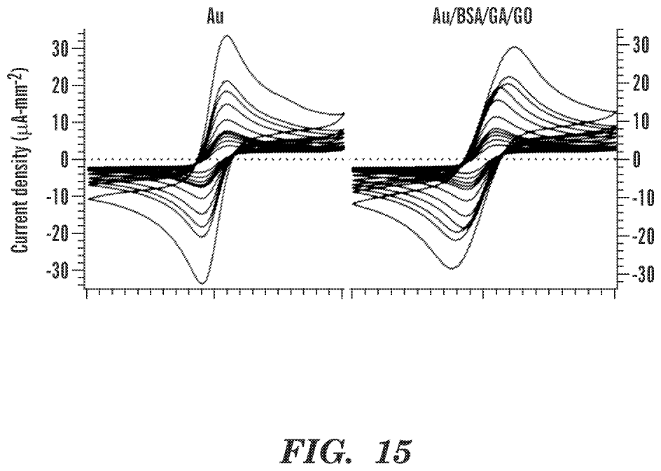

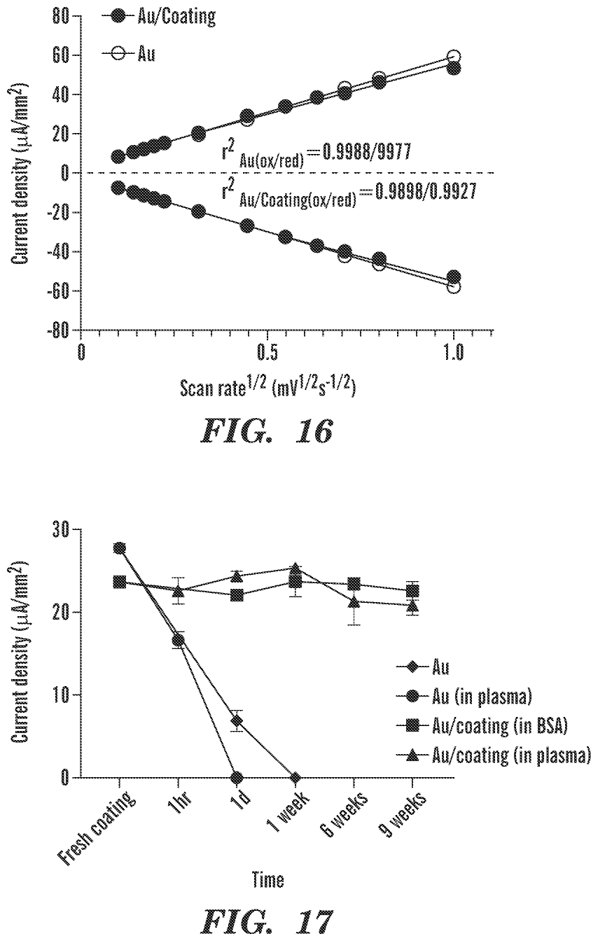

To demonstrate the comprehensive quality and state of solid-liquid interface and to assess the kinetics of electron transfer within the coating, electrode surface, and the solution, current density, as well as peak-to-peak separation (ΔEp) from this redox process was used. FIG. 13A is a plot showing optimization of concentration of prGOx. Bars are the current density of various nanocomposite with different concentrations of prGOx; fresh coating (black) and after 1-h exposure to 1% BSA (grey). FIG. 13B shows a typical CV showing oxidation and reduction peaks of an equimolar solution of 5 mM ferri-/ferrocyanide of various nanocomposite-coated electrodes. The coated gold electrodes maintained a similar current density (102%) compared to the bare gold electrode, while BSA/GA coatings resulted in a reduction of current density (36.3% of the fresh coating). BSA/GO coating maintained higher current density (84.5%) than BSA coating (32.6%) which may be due to nanoparticle-mediated electron transfer in BSA/GO. Sensors coated with various coatings were challenged by exposing to 1% BSA for 24 h. As expected, bare gold and different coatings (BSA, BSA/GA) exhibited reduced electrochemical performance except for the BSA/GO, which maintained a relatively high current density. Similarly, BSA and BSA/GA displayed broad ΔEp up to 0.443 V, indicating limited diffusion of ferri-/ferrocyanide to the electrode surface due to fouling. FIG. 14 is a plot showing electrochemical characterization of various electrode coatings. Bars are the mean current density of various nanocomposite-coated electrodes fresh (black) and after 1-d exposure to 1% BSA (grey). Even after 1-d of incubation in 1% BSA, the coating with BSA/prGOx/GA maintained a very high current density of 95.1% and low ΔEp of 185 mV. In addition, for assessing the mass transport of potassium ferri-/ferrocyanide with BSA/prGOx/GA coated electrodes, CV at different scan rates was evaluated. FIG. 15 shows CVs of bare gold-(left) and coated gold electrodes (right) of an equimolar solution of 5 mM fern-/ferrocyanide at different scan rates (0.01-1.0 V/s). The CV's at various incremental scan rate from mV/s to 1000 mV/s shows that anodic and cathodic peak current increased corresponding to the change in scan rate and exhibited excellent linear relationship, for both the oxidation (r² gold=0.998, r ² gold-coating=0.989) and reduction (r² gold=0.997, r ² gold-coating=0.992) current and square root of scan rate (FIG. 16) which indicates a surface-diffusion controlled redox electrode process. FIG. 16 shows a plot of extracted oxidation/reduction peak current (ip) mean values for gold electrode (black circles) and electrode with the coating (white circle) from the CV shown in FIG. 15 plotted versus the square root of the scan rate.

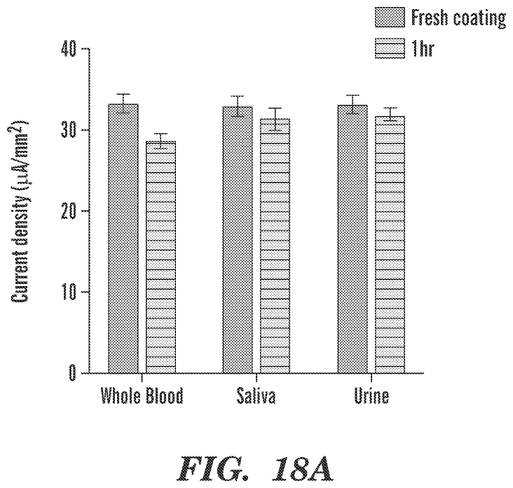

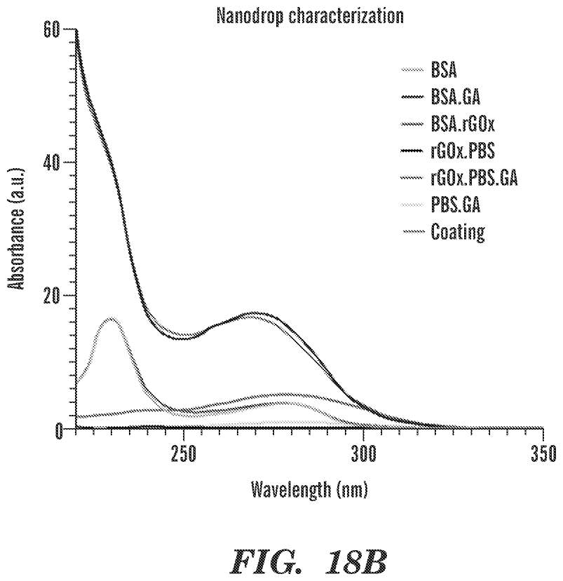

If the anti-fouling properties of a given strategy or nanocomposite are evaluated only towards one analyte/interferant, it limits the general assessment of how an anti-fouling strategy may work for the clinical diagnostics of various biomarkers. Therefore, the antifouling activity with complex biological fluids like plasma along with 1% BSA was evaluated. The rapidly coated nanocomposite showed excellent anti-fouling properties as shown by the high current density of the nanocomposite coated sensor both with 1% BSA (100.1% after one week and 95.5% after nine weeks) and plasma sample (107.2% after one week and 88.0% after nine weeks) as compared to the fresh bare gold electrode that showed a rapid decrease in current density to zero after one day in plasma. FIG. 17 shows a plotted comparison of antifouling activity with mean value of current density recorded at bare gold electrodes and Gold electrodes with antifouling coating stored for 9 weeks at 4° C. in 1% BSA and unprocessed human plasma. Red circles in FIG. 17 denote the final mean value of peak-to-peak distances. Statistical analysis in c, d, and e was performed using unpaired t-tests: *p<0.05, **P<0.01. The coated sensors were also challenged with whole blood, saliva, and urine for 1 hour without any significant reduction in current density. FIGS. 18A and 18B are plots characterizing the antifouling nanocomposite coating. Bar graph (18A) shows current density of fresh sensors (black bar) and sensors after 1 hour in varying biofluids: whole blood, saliva, and urine (grey bar). UV absorption spectra (18B) of BSA when mixed with/without GA and/or GOx. a.u., arbitrary units. Error bars represent the s.d. of the mean, n=3. Significance was determined by unpaired t-test (^{ns}P>0.05; *P<0.05; **P<0.01; all two tailed.

### Surface Characterization of Antifouling Coating.

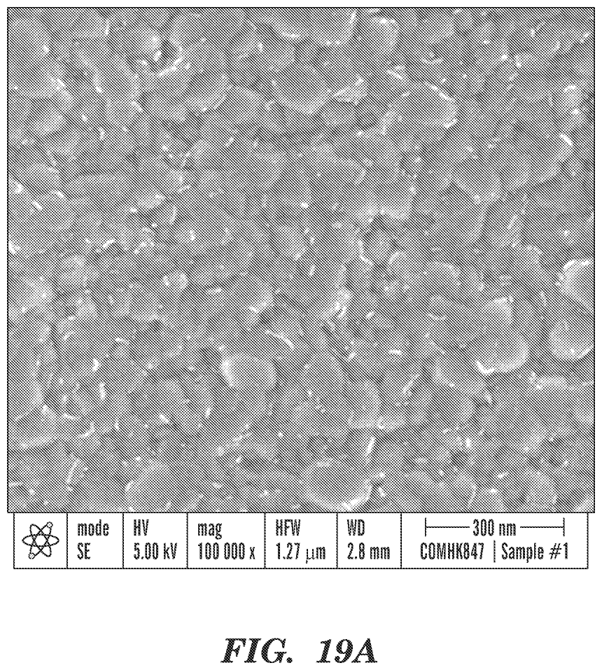

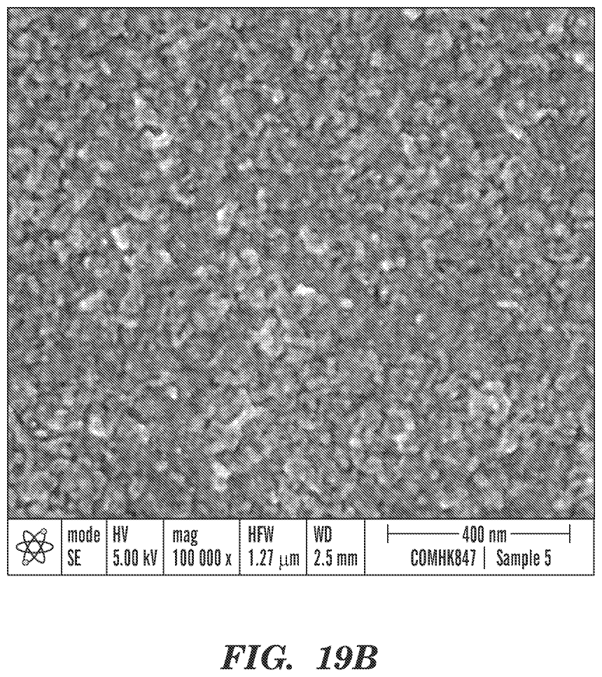

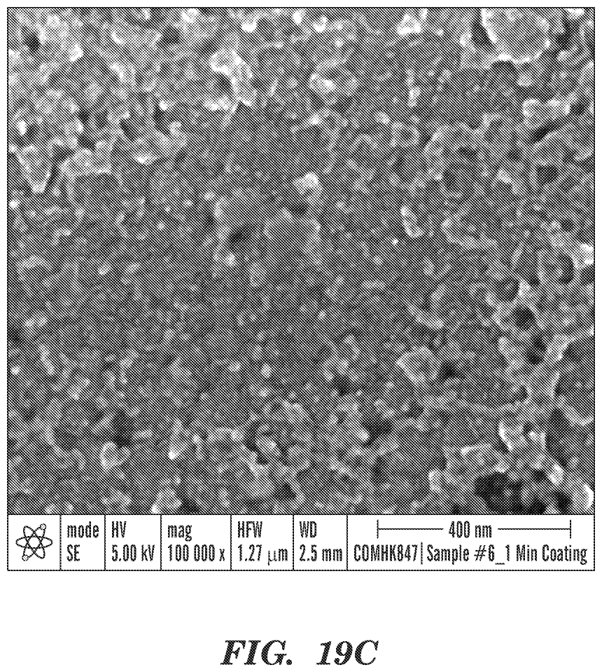

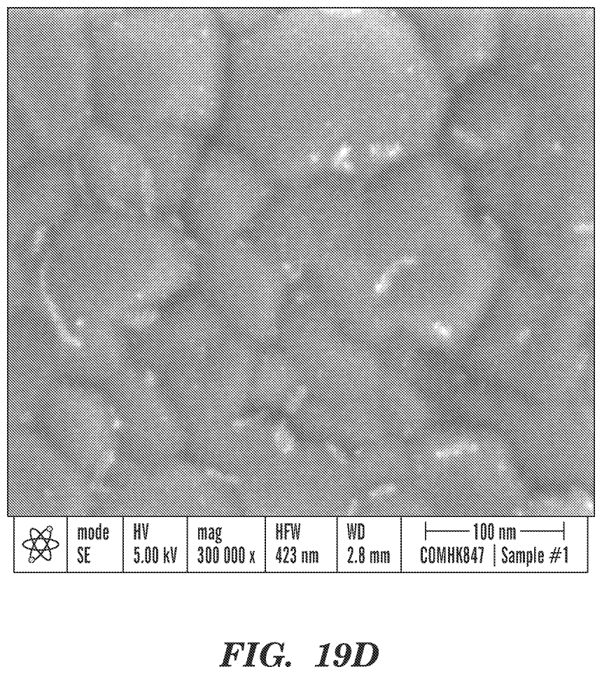

Different microscopic and spectroscopic characterization were performed to better understand the enhanced electrochemical properties of Gold/BSA/prGOx/GA coating to other coatings and bare gold electrodes. Scanning Electron Microscopy (SEM) image of the Gold/BSA/prGOx/GA shows a heterogenous sponge-like matrix but a visible highly porous structure which explains the surface-diffusion controlled redox electrode process as seen in bare gold, while Gold/BSA/GOx shows sponge-like structure without pores. FIG. 19A-19E are scanning electron micrograph of the bare Gold (19A, 19D), Gold/BSA/GOx (19B), and Gold/BSA/GOx/GA (anti-fouling coating) (19C, 19E).

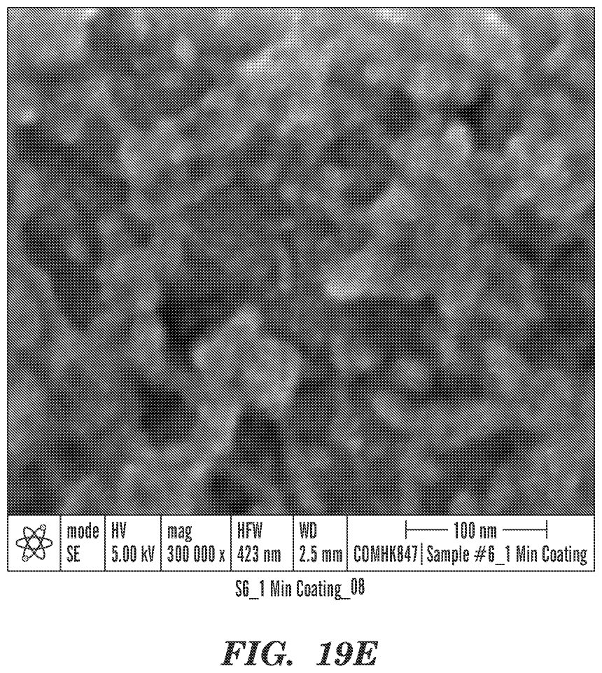

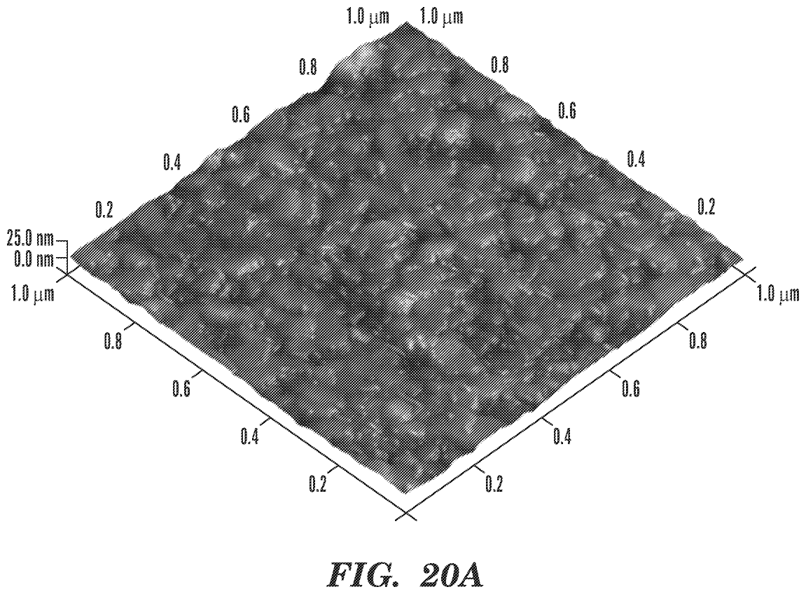

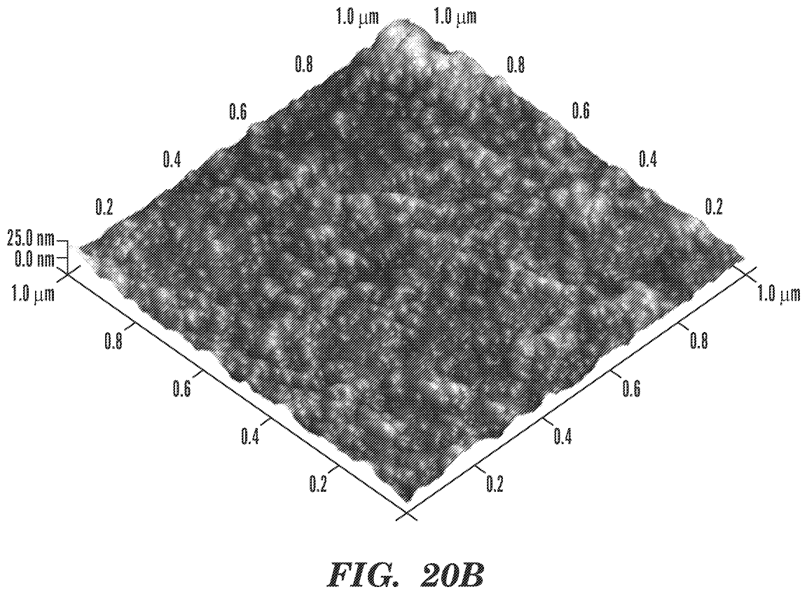

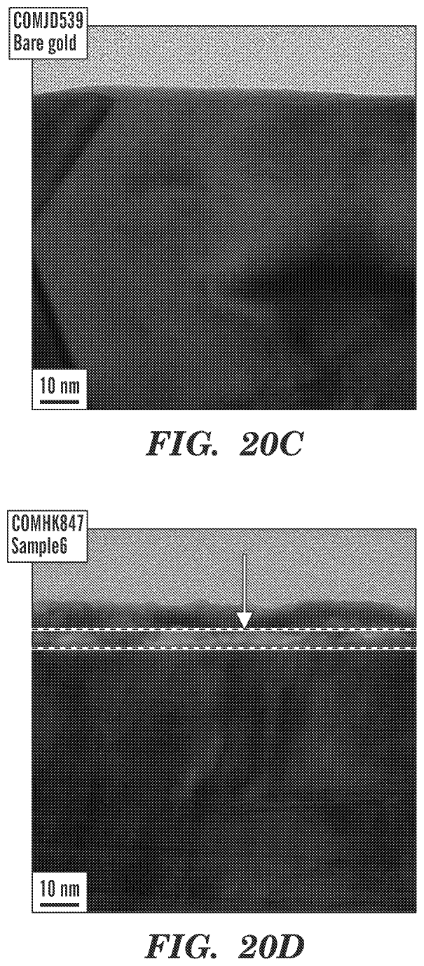

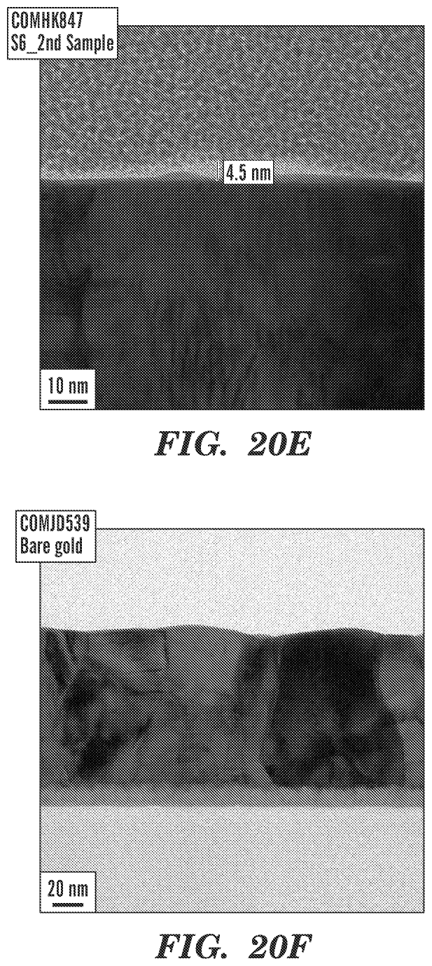

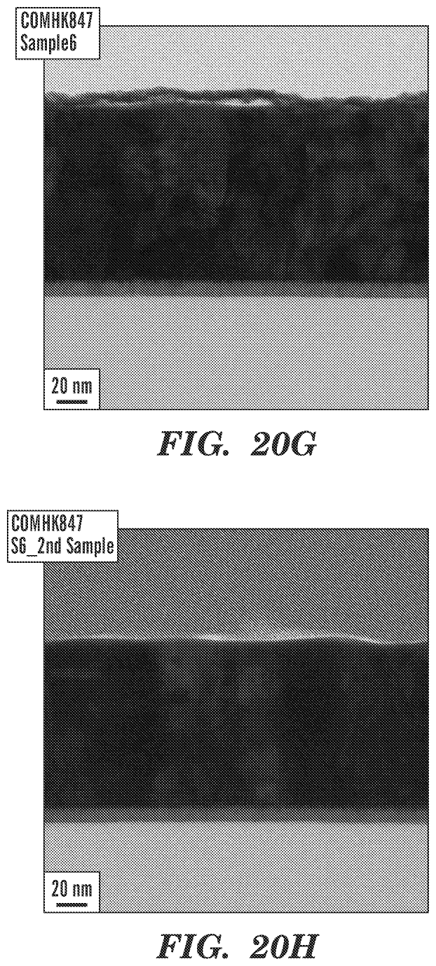

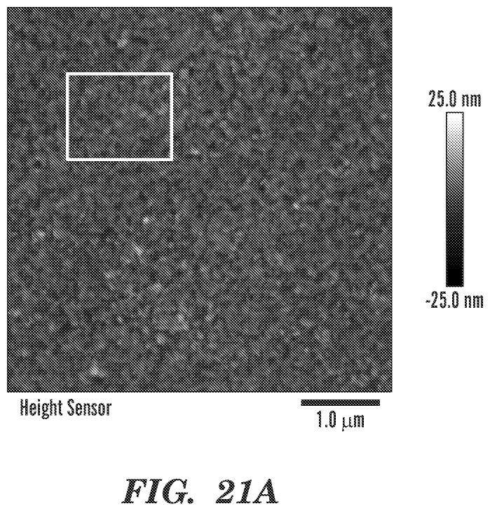

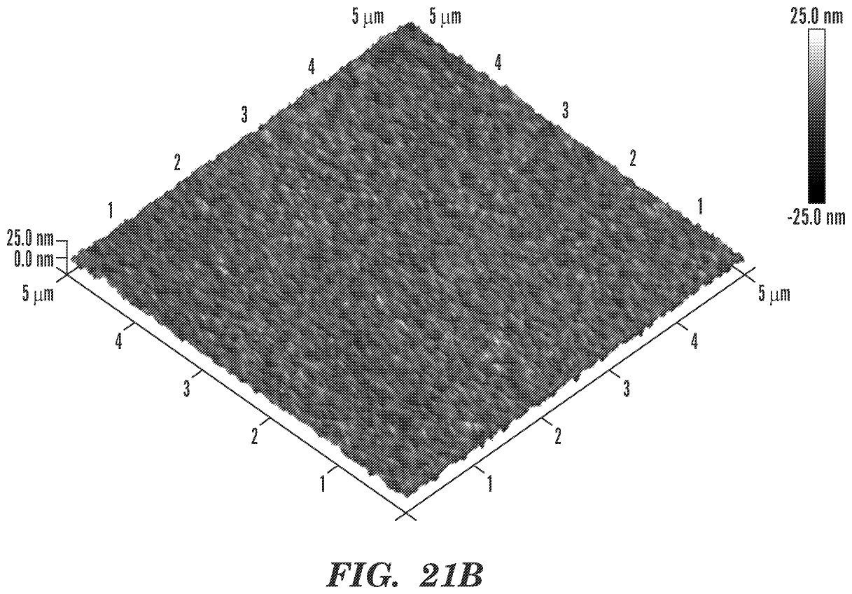

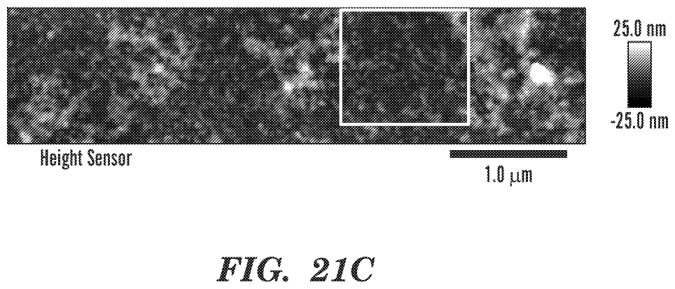

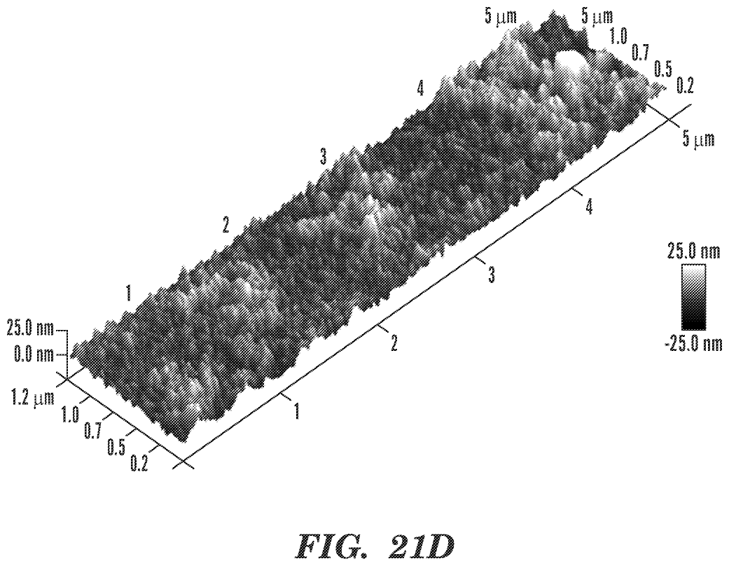

Atomic Force Microscopy (AFM) and TEM further confirmed the heterogeneity of the surface where a mean roughness of the coated surface (2.92 nm) was found to be higher than already rough bare gold (1.8 nm) as shown by Table 1, and which is also apparent from the AFM 3D survey images of bare gold and gold with the coating. FIG. 20A-20B illustrate the surface roughness characterized using Atomic Force Microscopy (AFM). FIG. 20A shows a 3D image of roughness for the bare gold electrode, and FIG. 20B shows a 3D image of coated gold electrode. FIG. 20C-20H show TEM images of bare gold (20C, 20F), coated gold electrode with top Iridium layer (20D, 20G), and without top Iridium layer (20E, 20H) for better contrast. FIG. 21A-21B show AFM 2D (21A) and 3D (21B) survey images of bare gold electrode, (5 mm×1.2 mm×50 nm), the blue square marks the approximate analysis location for the 1 mm×1 mm images. FIG. 21C-21D show AFM 2D (21C) and 3D (21D) survey images of gold electrode with antifouling coating (5 mm×1.2 mm×50 nm), the blue square marks the approximate analysis location for the 1 mm×1 mm images. These images illustrated heterogeneity leads to an increase in specific surfaces available for interaction with biomolecules and significant influence on the activity and concentration of surface-immobilized proteins.

TABLE 1
Roughness Results for AFM of bare gold and gold with coating.

| | | | | Surface Area |
|---|---|---|---|---|
| Sample | R_{q} (nm) | Rₐ (nm) | Rₘₐₓ (nm) | Diff (%) |
| Bare Gold | 2.32 | 1.8 | 22.9 | 2.61 |
| Coating | 3.73 | 2.92 | 27.5 | 5.15 |

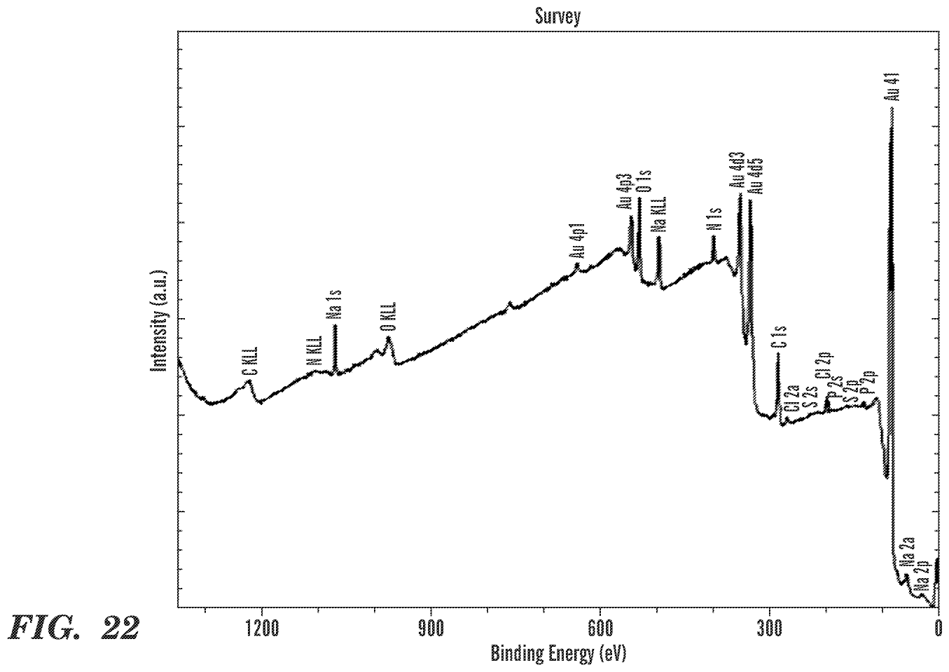

FIG. 22 shows an XPS spectra for gold electrode coated with antifouling coating. Table 2 shows concentration of elements as determined by XPS.

TABLE 2
Atomic Concentrations (in atomic %) Normalized to 100%
of the elements detected. XPS does not detect H or He.

| | C | N | O | Na | P | S | Cl | K | Au |
|---|---|---|---|---|---|---|---|---|---|
| Coating | 45.7 | 8.1 | 17.5 | 7.8 | 1.6 | 0.5 | 2.6 | 0.8 | 15.4 |

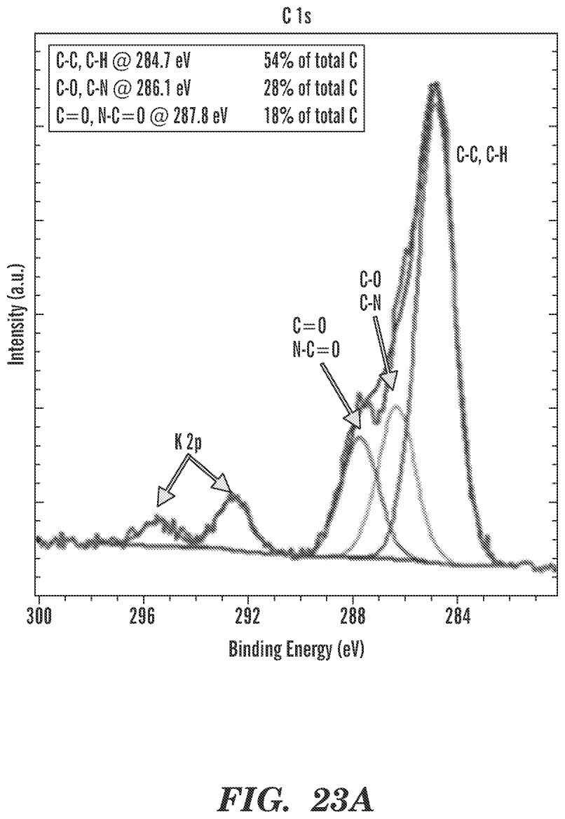

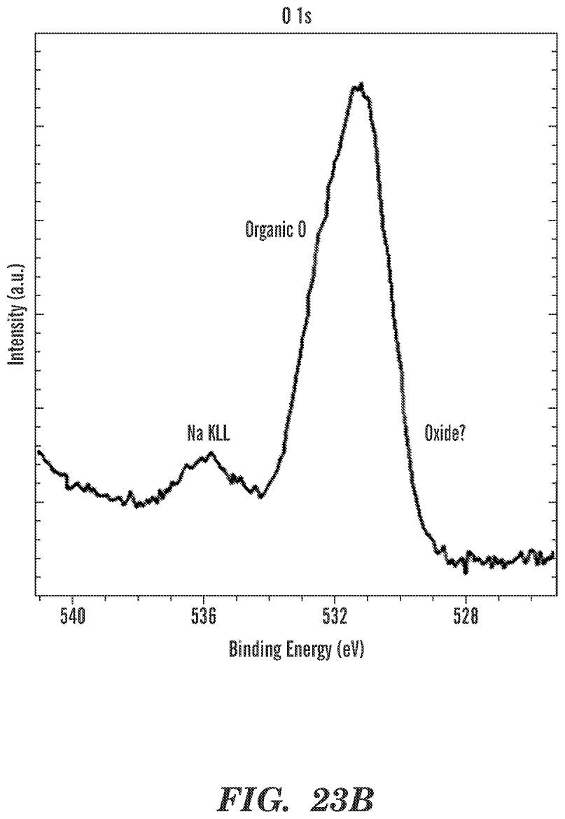

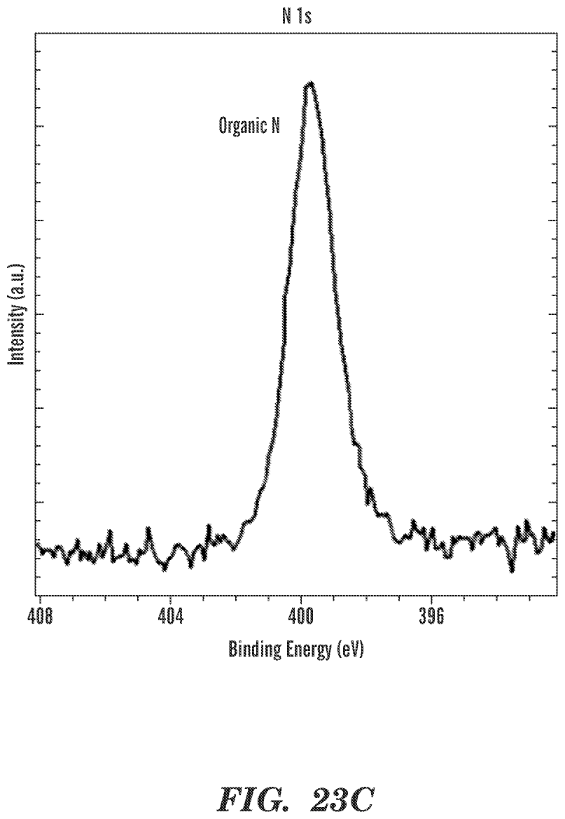

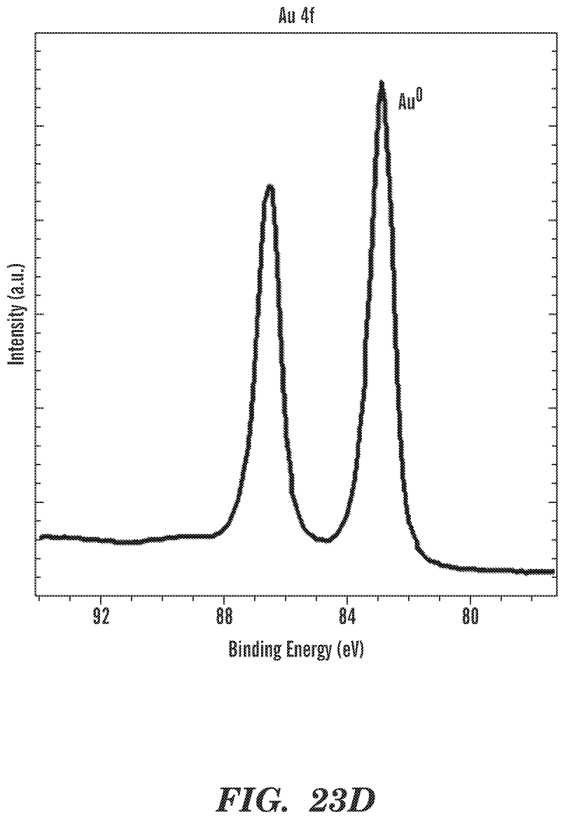

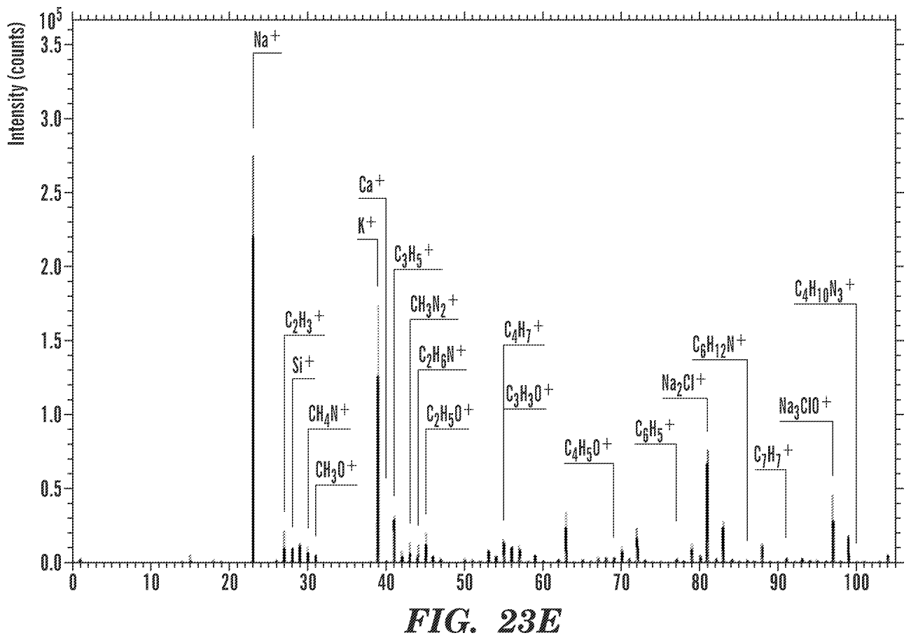

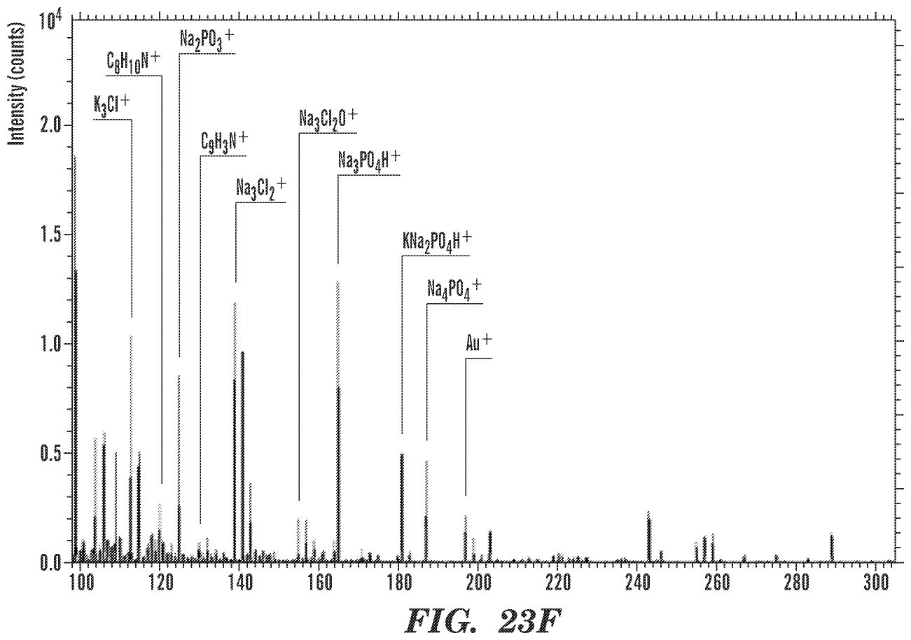

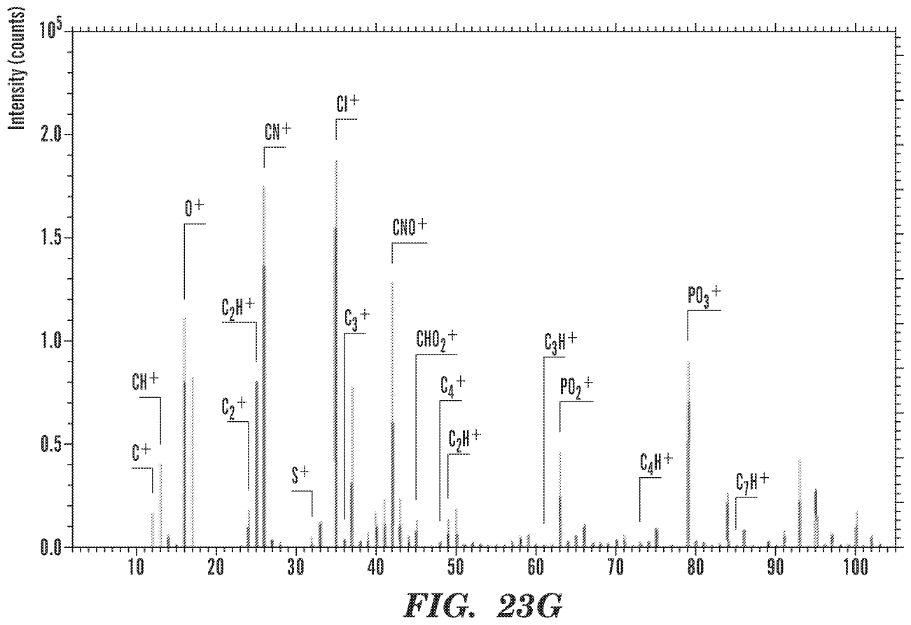

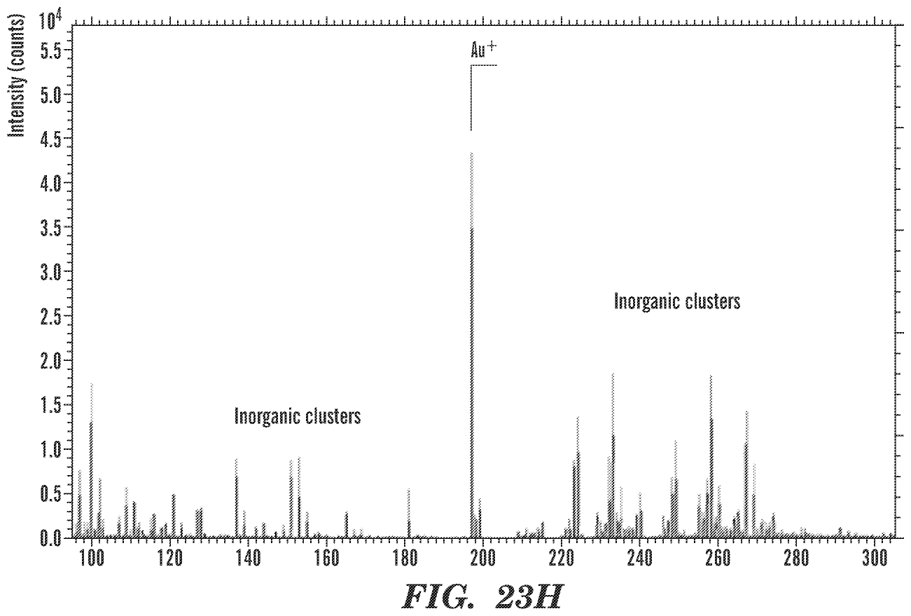

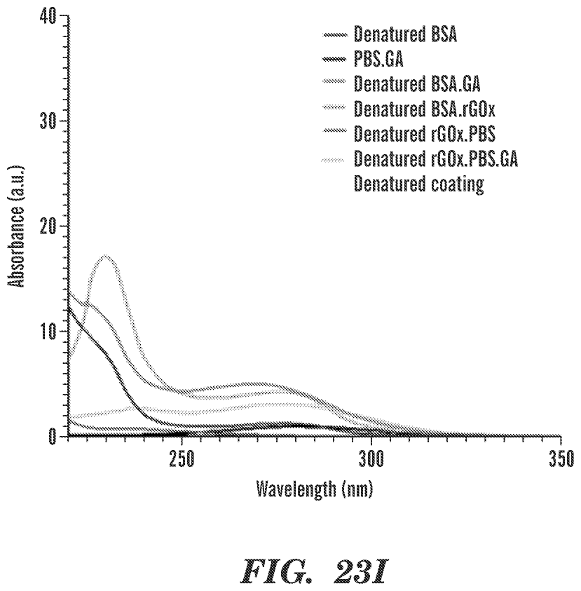

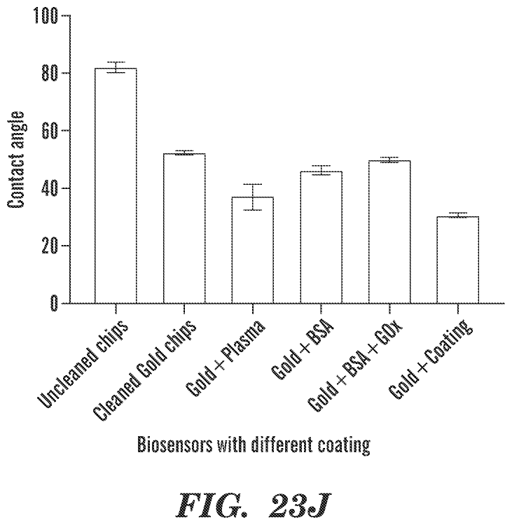

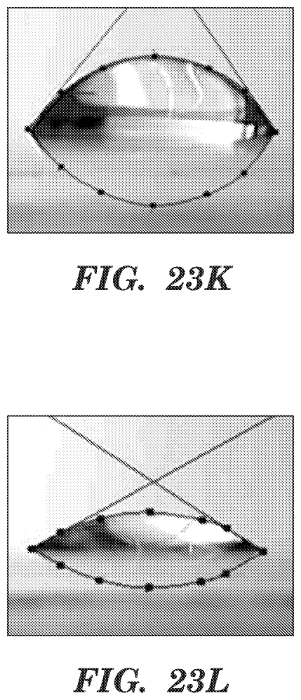

The product of the rapid cross-linking mechanism results in porous 3D molecular networks, and the reaction can be observed by the increase in absorbance at 265-270 nm. As expected, and as by FIG. 18B, an increase in absorbance at 265-270 nm for both BSA-GA and coating solution can be ascribed to the crosslinking of BSA and GA. However, because of denaturation of the solution during coating, minimum crosslinking can be seen between BSA-GA (FIG. 23I), but the coating solution (BSA/prGOX/GA) still shows an increase in absorbance at 265-270 nm, indicating high crosslinking of the coating solution. The antifouling coating also increases the sensor's hydrophilicity compared to Gold/BSA and GolcVBSA/GOx (FIG. 23K-23L, 25A-25F), which facilities the binding of capture antibody. TEM of the bare gold (FIG. 20C, 20F) and Gold/coating (FIG. 20D, 20E, 20G, 20H) was performed to estimate the thickness of the coating layer to be around 4.5 nm.

FIG. 23A-23L depict the characterization of the antifouling nanocomposite coating. X-ray Photoelectron Spectroscopy (XPS) spectrum of the gold electrode with antifouling coating for C1s peak (23A), O1s peak (23B), N1 s peak (23C), and Au4f peak (23D). Time-of-Flight Secondary Ion Mass Spectrometry (TOF-SIMS) spectra of the gold electrode with antifouling coating for positive ions (23E) and (23F) and negative ions (23G) and (23H). UV absorption spectra of denatured BSA when mixed with/without GA and/or GOx. a.u., arbitrary units (231). Contact angle of the EC-Biosensor before and after cleaning and plasma treatment with different kinds of coatings (23J). Contact angle measurement for cleaned Gold chips (23K) and Gold chip with the anti-fouling coating (23L).

As described in the preceding, the chemical morphology of the Gold/BSA/prGOx/GA coating was also evaluated using X-ray photoelectron microscopy (XPS) and Time-of-Flight Secondary Ion Mass Spectrometry (TOF-SIMS). XPS spectrum shows the data with C in the proportion shown in FIG. 23A and Table 3. The peak of C(1s) moves from 284 eV and focuses around 284.7 eV, corresponding to the overlap of C—C, C—H, C═C bonds. C(1s) centered peak around 288 eV with two binding energy, 287.8 (C═O, N—C═O bond) and 286.1 (C—O, C—N bond), which could be assigned to overlap chemical groups such as [R—CH2-NH—(C*O)—] and [(R—CH2*—NH—(CO)—] respectively characteristics of protein and overlap (C═N, C—N and C—O bonds).35-37 For the binding energy of N(1s) spectrum, FIG. 23C, a central peak around 400 eV corresponding to pure N(1s) peak at 399 eV along with peaks at 398.3, 398.8, 399.8, and 400.8 eV which corresponds to C—N, C═N, and NO bonds, respectively were found. The high-resolution spectrum for the Au4f appears as two doublets, located in the energy range characteristic for Au₂O₃ and Au (FIG. 23D) and O (1s) (FIG. 23B) around 531 eV. Based upon this data we can suggest that the BSA/prGOx/GA is successfully immobilized to the gold electrode of the sensor. TOF-SIMS was performed for depth profile analysis of gold electrode coated with antifouling nanocomposite. Ions characteristic of BSA including CH₄N+, CH₃N₂+, C₂H₆N+, C₄H₅O+, C₄H₈N+, C₃H₈NO+, C₅H₁₂N+, C₃H₈NO+, C₅H₁₂N+, C₄H₁₀N₃+, C₈H₁₀N+, and C₉H₈N+ were observed (FIGS. 23E and 23F). Additional species observed include hydrocarbon species, aromatic species, and small oxygen-containing organic ions, including C−, CH−, C₂−, C₂H−, C₃−, C₄−, C₄H−, C₅H−, C₇H−, CN−, and CNO− as also seen in the literature indicating the presence of graphene oxide and BSA (FIGS. 23G and 23H).

TABLE 3
Carbon Chemical States (in % of Total C) Values in this
table are percentages of the total atomic concentration
of the corresponding element shown in Table 2.

| | C—C, C—H | C—O, C—N | C═O, N—C═O |
|---|---|---|---|
| Coating | 63 | 21 | 16 |

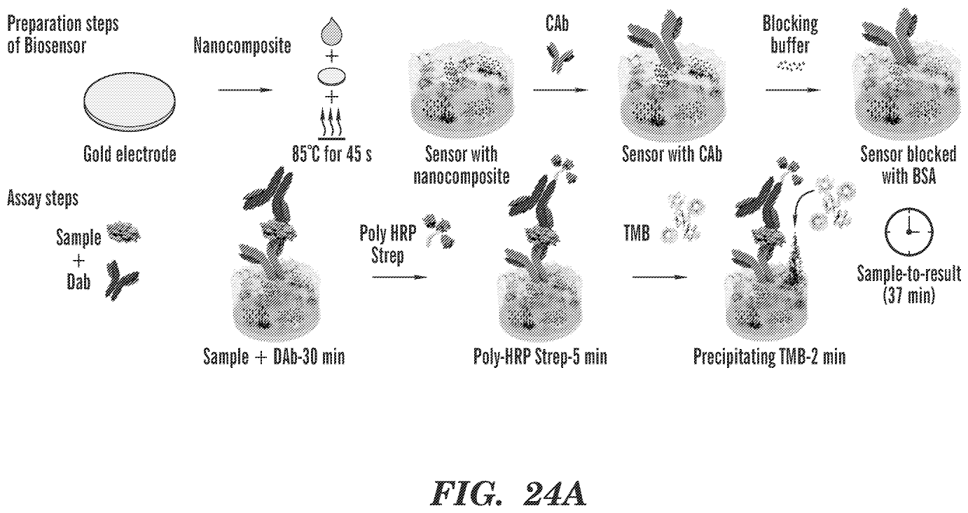

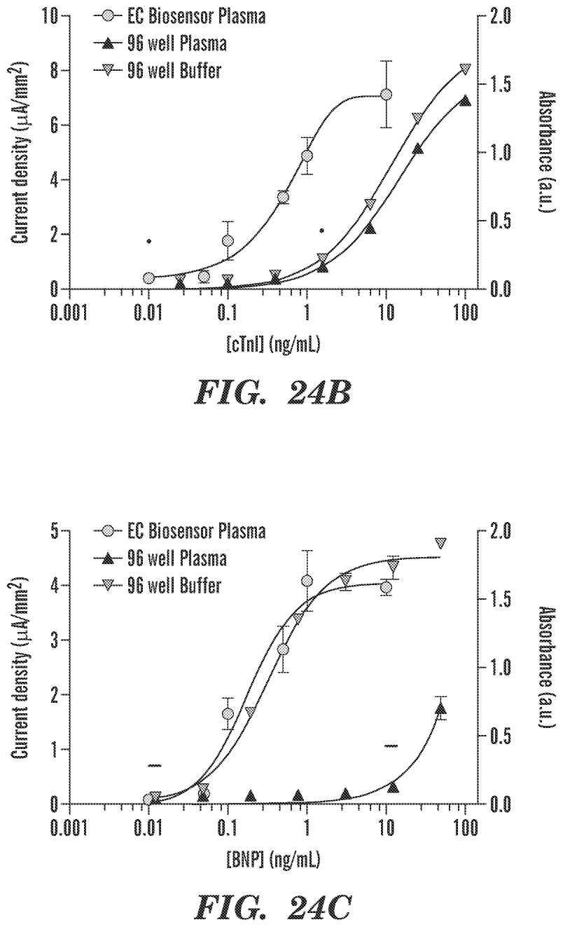

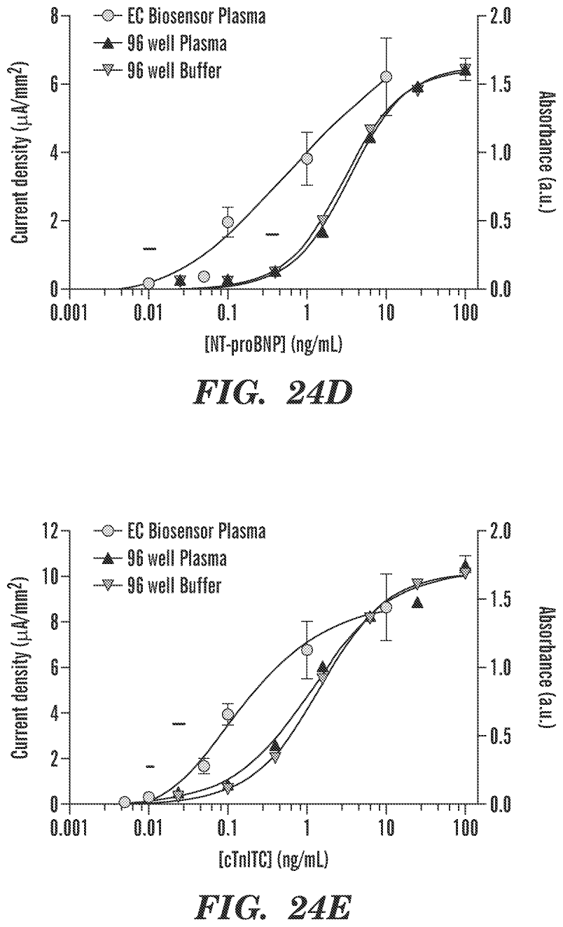

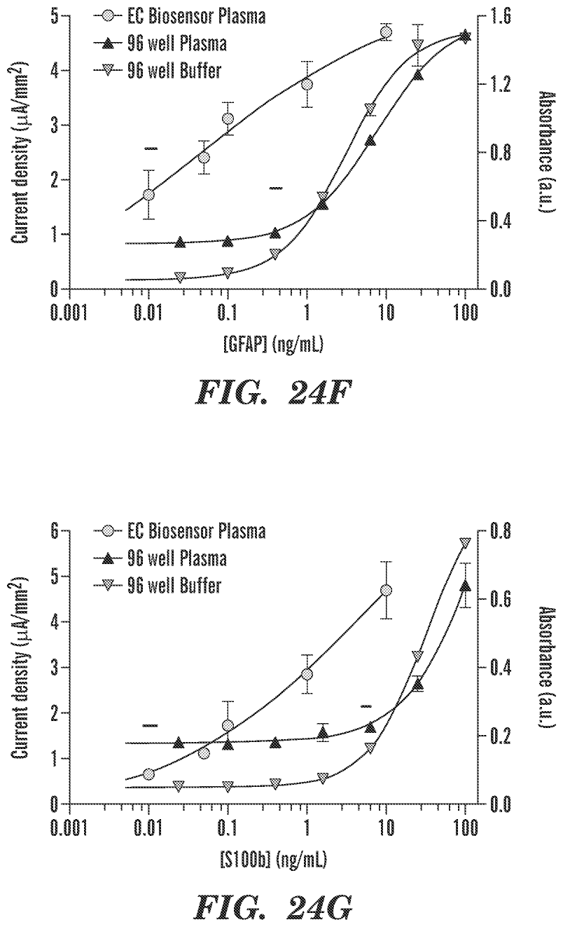

FIG. 24A-24G show schematics and calibration curves for MI and TBI biomarkers using EC-biosensors and 96 well plate. FIG. 24A shows a schematic for the preparation and assay steps for the EC-Biosensor. FIG. 24B-24G are calibration curves for different biomarkers. The left y-axis shows current density for different concentrations of biomarkers run on EC-Biosensors (red circle) using unprocessed human plasma while the right y-axis shows mean absorbance (a.u) for different concentration of biomarkers run in 96 well plate using plasma (black triangle) and 1% BSA in PBS (blue triangle). Different biomarkers tested include (24B) cardiac troponin I (cTnI); n=6, (24C) B-type natriuretic peptide (BNP); n=4, (24D) N-terminal (NT)-pro hormone BNP (NT-proBNP); n=4, (24E) cardiac troponin ITC complex (cTnITC); n=5, (24F) Glial fibrillary acidic protein (GFAP); n=3, and (24G) S100b protein; n=3. Error bars represent the s. d. of the mean; n=2 for all 96 well assays. Analysis was done using 4-Parameter Logistic (4PL) curve fitting. Significance was done against background (0 ng/mL) and determined by unpaired t-test (^{ns}P>0.05; *P<0.05; **P<0.01***P<0.001; ****P<0.0001; all two-tailed.

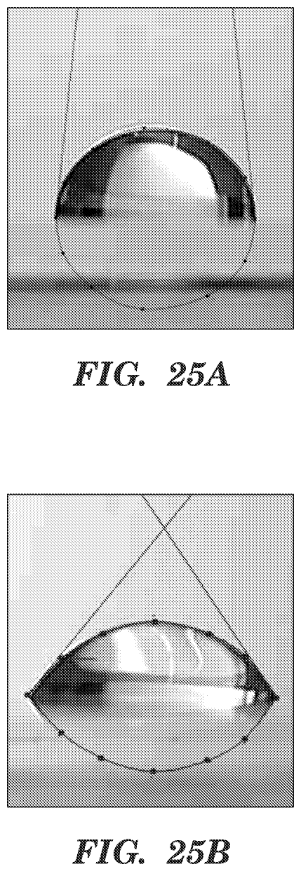

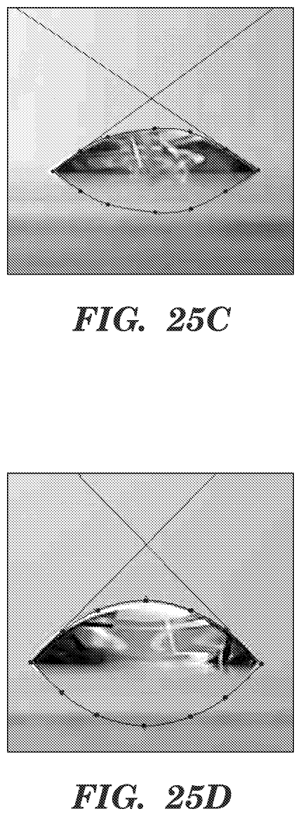

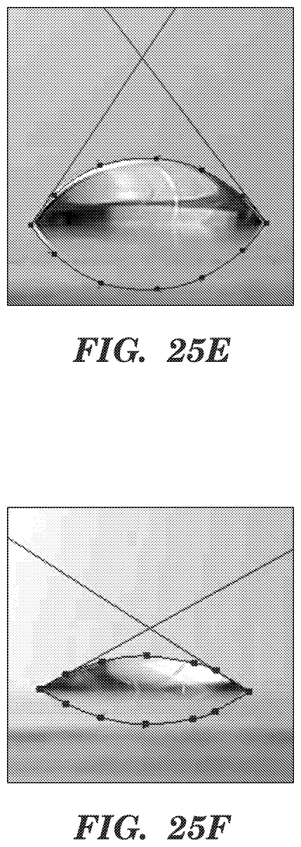

FIG. 25A-25F are images of a drop on a chip for contact angle measurements. FIG. is an uncleaned chip with protective organic layer. FIG. 25B is a chip cleaned with acetone and Isopropyl alcohol. FIG. 25C is a plasma treated chip. FIG. 25D is a plasma-treated chips with BSA coating. FIG. 25E is a plasma-treated chip with BSA and GOx coating. FIG. 25F is a plasma-treated chips with antifouling nanocomposite coating.

### Ultra-Sensitive Analytical Performance of the EC-Biosensor.

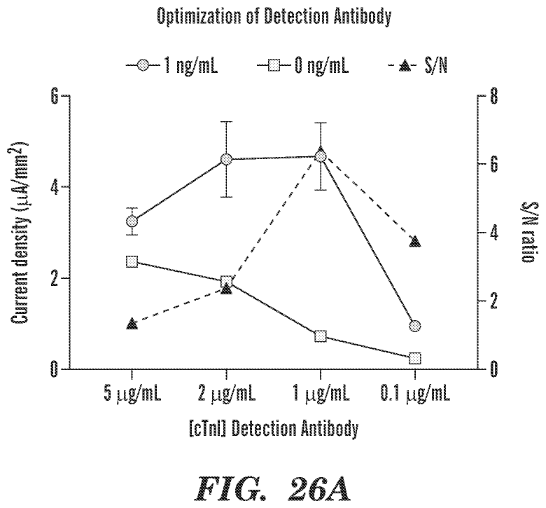

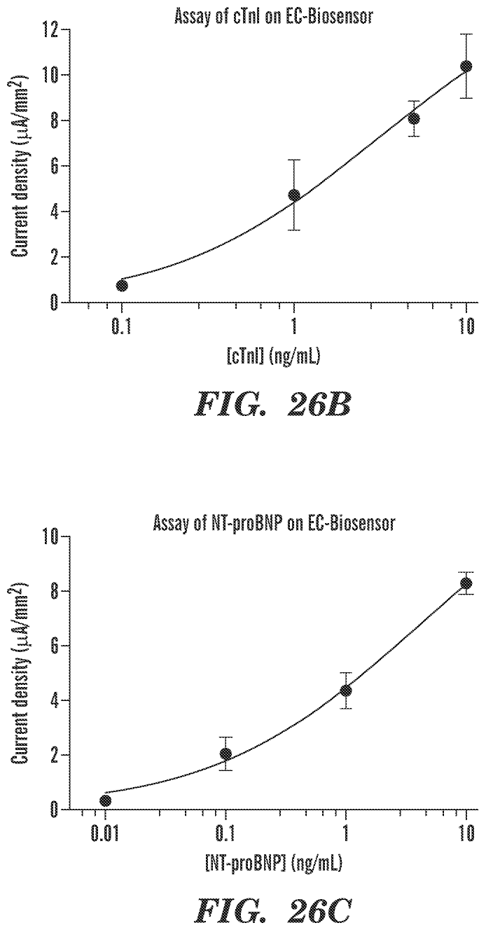

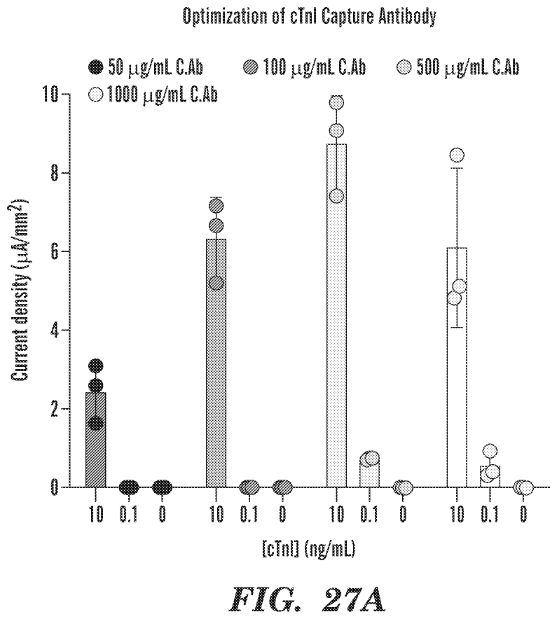

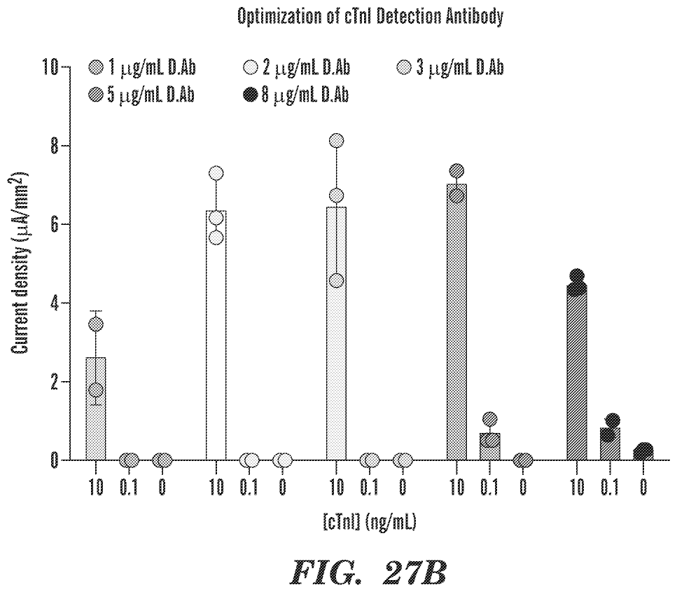

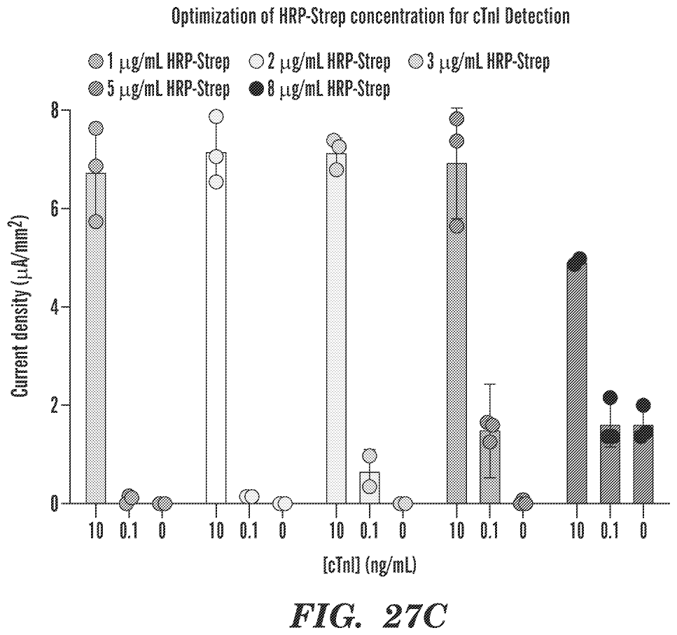

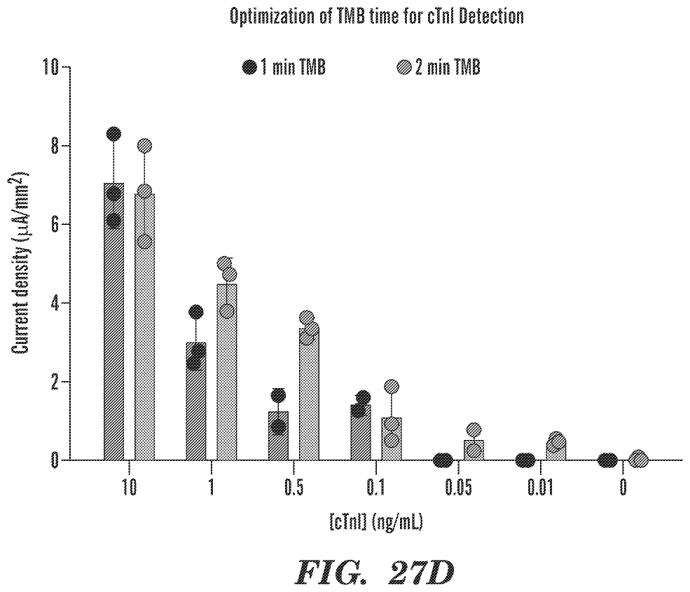

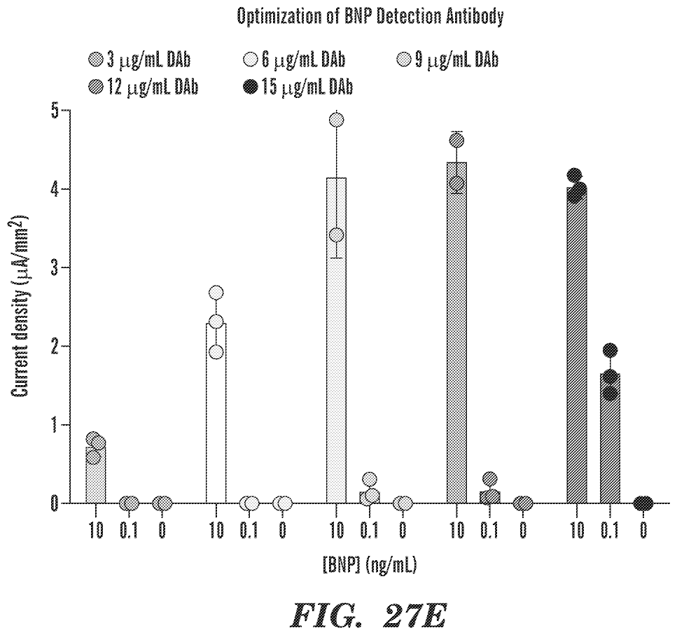

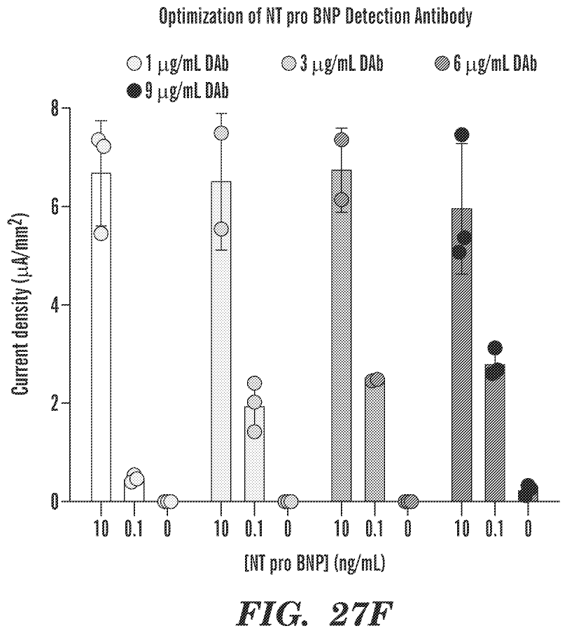

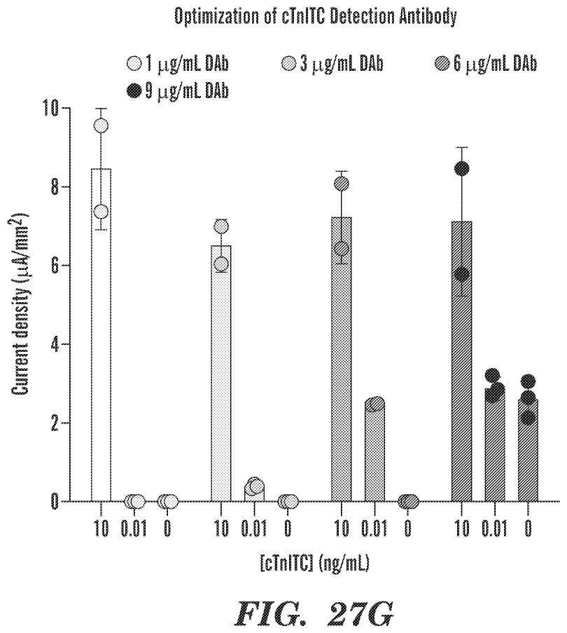

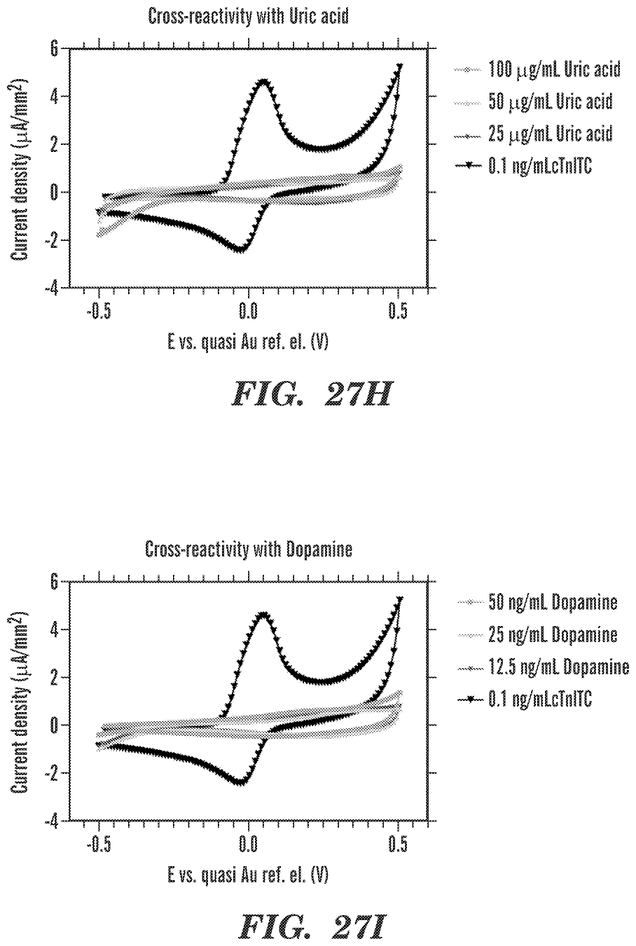

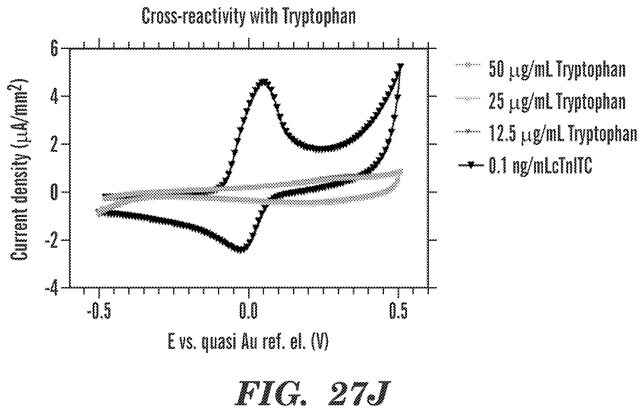

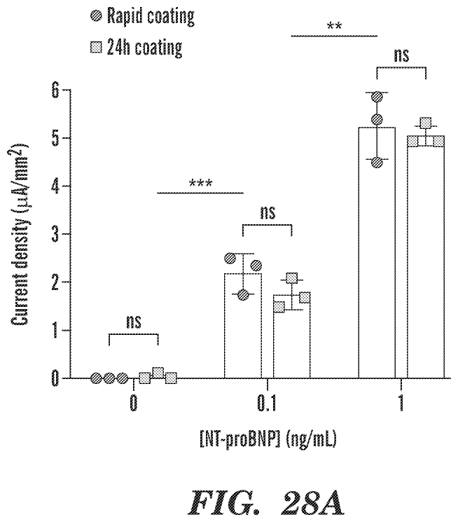

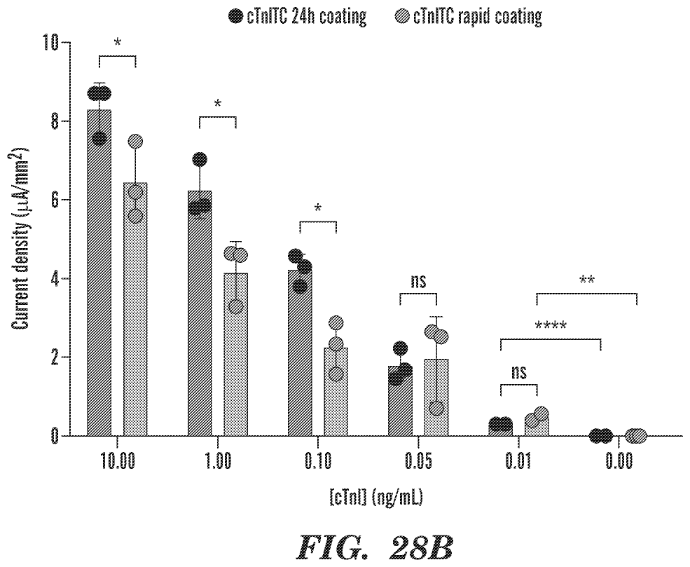

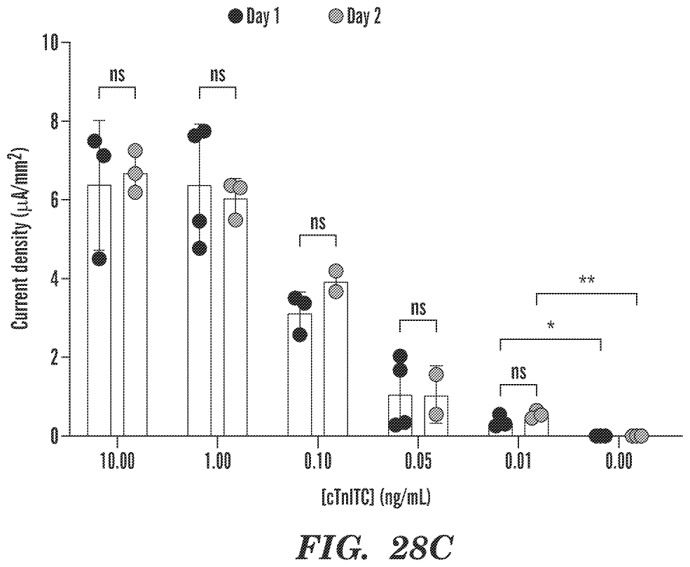

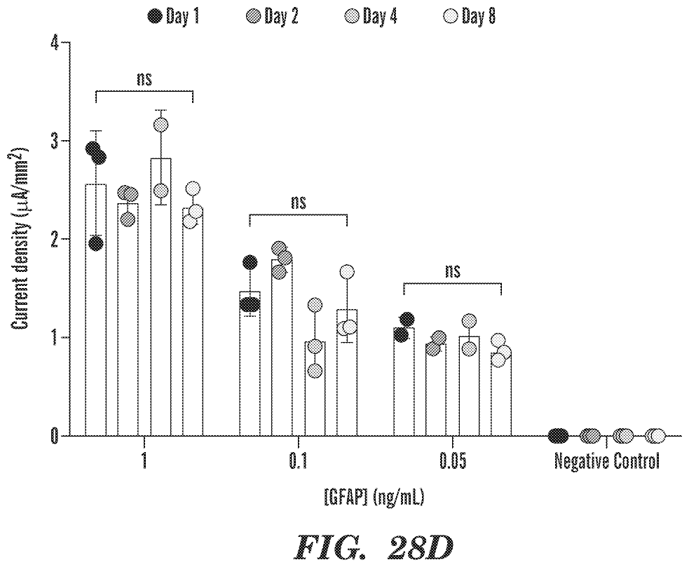

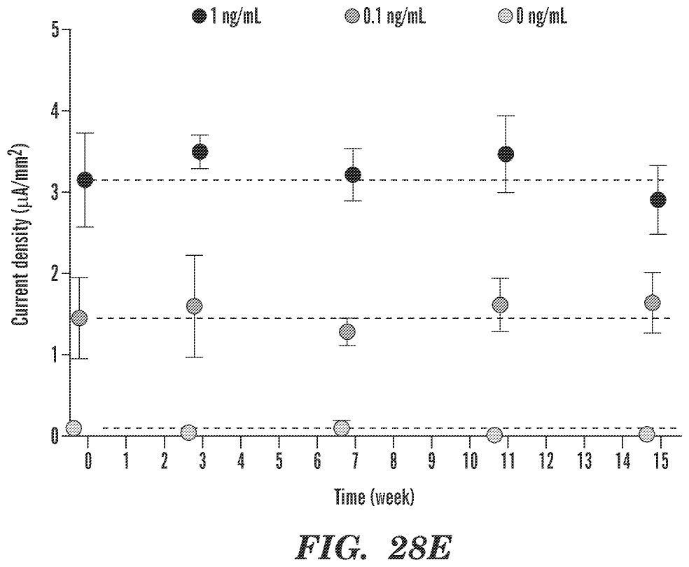

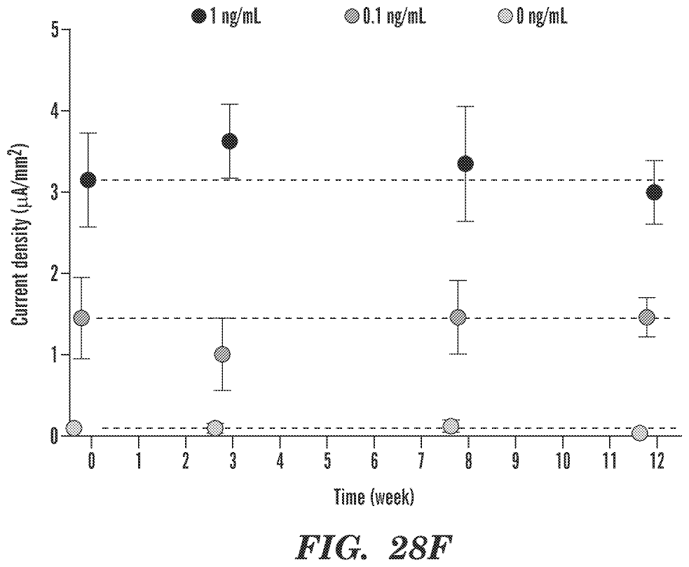

Using the nanocomposite coating, ultra-high sensitivity and specificity were achieved by optimizing different experimental conditions, including the concentration of capture and detection antibody, poly-streptavidin-HRP, and TMB time. Initially, a two-step assay of cTnI and NT-proBNP was performed where the sample was added, followed by an optimized concentration of 1 μg/mL detection antibody with a sensitivity of 24 and 3 pg/mL, respectively but the assay required 1 h 21 min (FIG. 26A-26C). In a single-step assay, sample mixed with detection antibody was exposed to the EC-Biosensor for 30 min reducing the total assay time to 36 min. The capture and detection antibody for the assay was optimized to meet the clinical cut-off range of each biomarker, while poly streptavidin HRP (5 ug/mL) and TMB timing (2 min) was optimized based on troponin I considering the high sensitivity required for troponin I assay (FIG. 27A-27G). It is noted that the EC-Biosensor showed ultra-high selectivity with no background signal for commonly found interfering molecules in biological samples like uric acid, dopamine, and tryptophan, as shown by FIG. 27H-28J).

FIG. 26A-26C illustrates optimization and two-step assay for different biomarkers. FIG. 26A shows optimization of detection antibody for the assay of cTnI. Left y-axis show current density for 1 ng/mL (blue) and 0 ng/mL (green) of cTnITC while right y-axis shows signal to noise ratio at different concentration of anti-cTnI detection antibody. FIG. 26B is a calibration curve of cTnI run on the EC Biosensor with antifouling coating using two-step assay and optimized detection antibody concentration. FIG. 26C is a calibration curve of NT-proBNP run on the EC Biosensor with antifouling coating using two-step assay and similar assay conditions. Error bars represent the s.d. of the mean, n=3.

FIG. 27A-27G illustrates assay development and optimization for single step assay for different biomarkers. FIG. 27A shows optimization of cTnI capture antibody. Bar graph shows the mean current density for different concentration of capture antibody (50, 100, 500, and 1000 μg/mL) to perform assay of cTnI at 3 different concentrations (10, 0.1, and 0 ng/mL). FIG. 27B shows optimization of cTnI detection antibody. Bar graph shows the mean current density for different concentration of detection antibody (1, 2, 3, 5, and 8 μg/mL) to perform assay of cTnI at 3 different concentrations (10, 0.1, and 0 ng/mL). FIG. 27C shows optimization of HRP-Streptavidin. Bar graph shows the mean current density for different concentration of HRP-Streptavidin (1, 2, 3, 5, and 8 μg/mL) to perform assay of cTnI at 3 different concentrations (10, and 0 ng/mL). FIG. 27D shows optimization of TMB incubation time. Bar graph shows the mean current density for different incubation time for TMB (1 and 2 min) to perform assay of cTnI at different concentrations (10, 1, 0.5, 0.1, 0.05, 0.01, and 0 ng/mL). FIG. 27E shows optimization of BNP detection antibody. Bar graph shows the mean current density for different concentration of detection antibody (3, 6, 9, 12, and 15 μg/mL) to perform assay of BNP at 3 different concentrations (10, 0.1, and 0 ng/mL). FIG. 27F shows optimization of NT-proBNP detection antibody. Bar graph shows the mean current density for different concentration of detection antibody (1, 3, 6, and 9 μg/mL) to perform assay of NT-proBNP at 3 different concentrations (10, and 0 ng/mL). FIG. 27G shows optimization of cTnITC detection antibody. Bar graph shows the mean current density for different concentration of detection antibody (1, 3, 6, and 9 μg/mL) to perform assay of cTnI at 3 different concentrations (1, 0.01, and 0 ng/mL). Error bars represent the s.d. of the mean; n=3.

FIG. 27H-27J illustrate cross-reactivity test of the EC Biosensor. CV oxidation and reduction peaks of uric acid (27H), Dopamine (27I), and Tryptophan (27J) at 3 different concentrations (at, higher, and lower than physiological level) along with cTnITC at 0.1 ng/mL

FIG. 28A-28F shows characterization and stability of antifouling nanocomposite and precipitated TMB. FIG. 28A shows an assay of NT-proBNP comparing the mean current density for different concentrations of NT-proBNP done on EC-Biosensor with rapid coating (blue) and 24 h coating (red). FIG. 28B depicts a comparison of rapid versus 24 h coating for assay of cTnI. Bars are the mean current density for cTnI done on EC-Biosensor with 24 h coating (black dots) and EC-Biosensor with rapid coating (blue dots); n=3. FIG. 28C shows stability of precipitated TMB for detection of cTnITC. Bars graph shows the mean current density for cTnITC measured just after the assay, day 1 (black dot; n=4) and 1 day after the assay, day 2 (blue dot; n=3). FIG. 28D shows stability of precipitated TMB for detection of GFAP. Bars graph shows the mean current density for GFAP measured just after the assay, day 1 (black dot), day 2 (blue dot), day 4 (red dot), and day 8 (orange dot); n=3. FIG. 28E shows stability of coating solution stored at 4 degrees. The line graph shows the mean current density for the assay of GFAP on EC biosensor coated with nanocomposite stored over 15 weeks; n=3. FIG. 28F shows stability of coating solution stored at room temperature. The line graph shows the mean current density for the assay of GFAP on EC-Biosensor coated with nanocomposite stored over 12 weeks; n=3. Error bars represent the s.d. of the mean. In a, b, c significance was determined by multiple/unpaired t-test (^{ns}P)>0.05; *P<0.05; **P<0.01 ***P<0.001; ****P<0.0001; all two-tailed. In d, Two-way ANOVA was used to observe a significant source of variation, ^{ns}P>0.05.

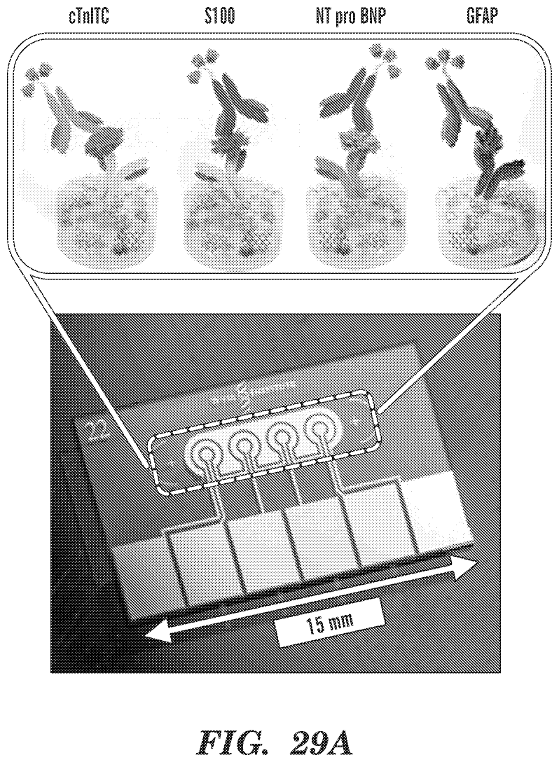

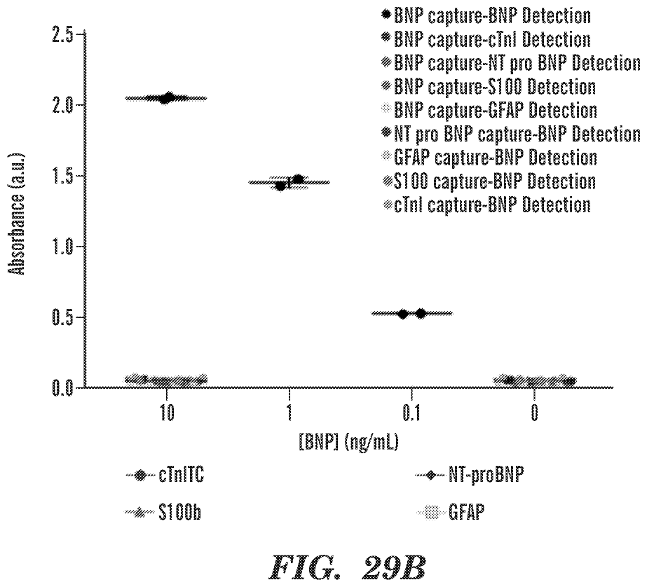

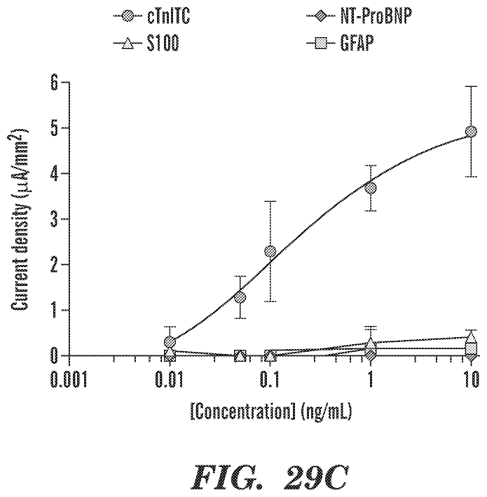

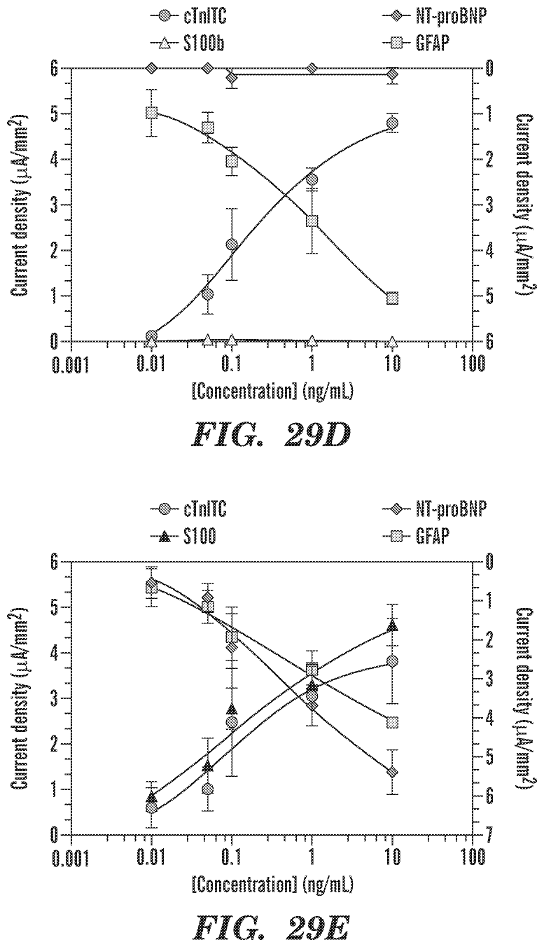

FIG. 29A-29E illustrates the specificity and Multiplexed detection for MI and TBI Biomarkers using EC-Biosensors. FIG. 29A shows a schematic for multiplexed detection on the EC-Biosensor showing detection of four biomarkers in a single EC-Biosensor. FIG. 29B shows Specificity and cross-reactivity of BNP antigen against different capture antibodies (anti-cTnITC, anti-NT-proBNP, anti-GFAP, and anti-S-100b) and detection antibodies (anti-cTnITC, anti-NT-proBNP, anti-GFAP, and anti-S-100b) along with specific detection with anti-BNP capture and detection antibody at different concentrations of BNP done in 96 well plate. FIG. 29C shows a calibration curve for multiplex detection of cTnITC on the EC-Biosensor with four different capture antibodies on each electrode (anti-cTnITC, anti-S-100b, anti-GFAP, and anti-NT-proBNP). FIG. 29D shows a calibration curve for multiplex detection of increasing concentrations of cTnITC (left y-axis) and decreasing concentrations of GFAP (right y-axis) on the EC-Biosensor with four different capture antibodies on each electrode. FIG. 29E shows a calibration curve for multiplex detection of increasing concentrations of cTnITC and S-100b (left y-axis) and decreasing concentrations of GFAP and NT-proBNP (right y-axis) on the EC-Biosensor with four different capture antibodies on each electrode. Error bars represent the s.d. of the mean, n=2 for 96 well plate, n=3 for EC-Biosensors.

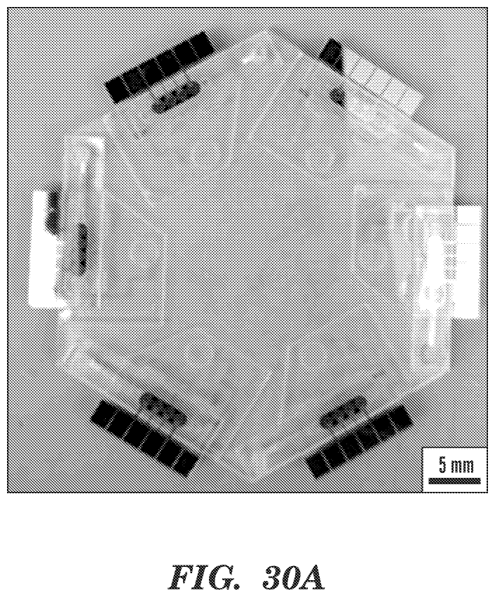

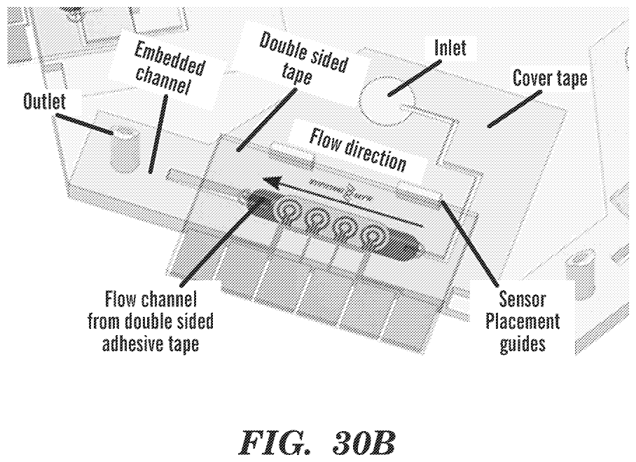

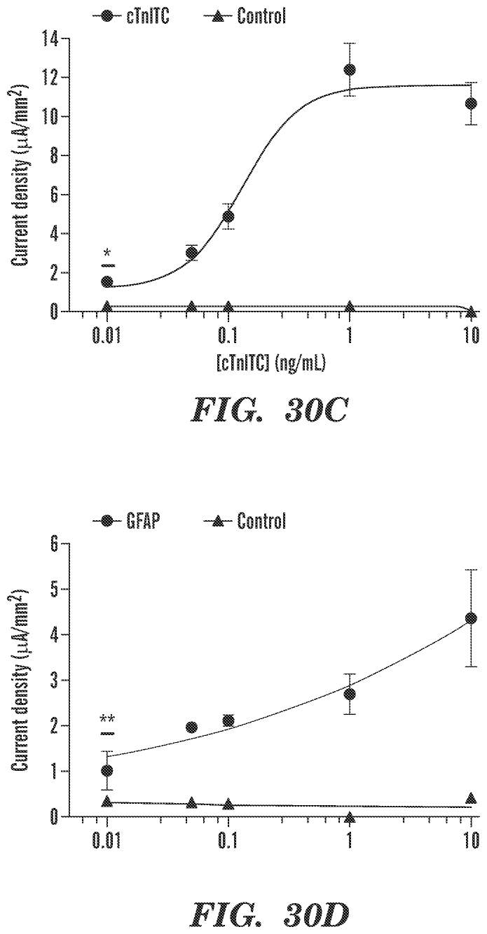

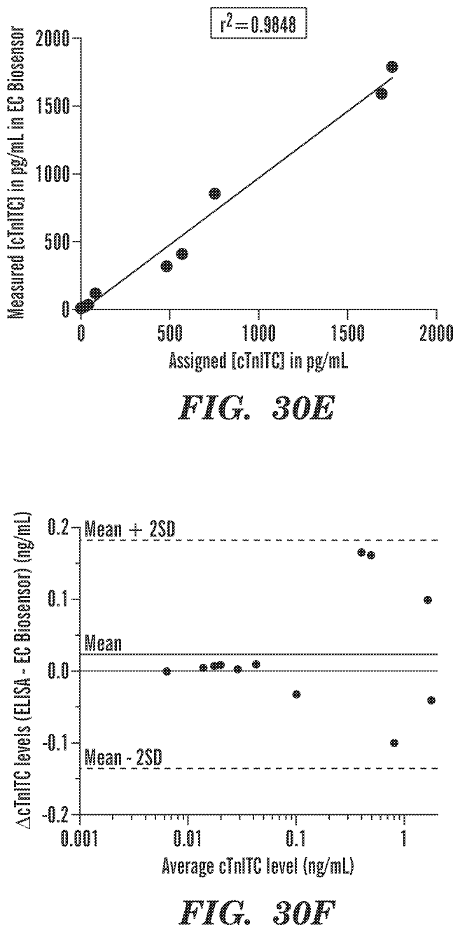

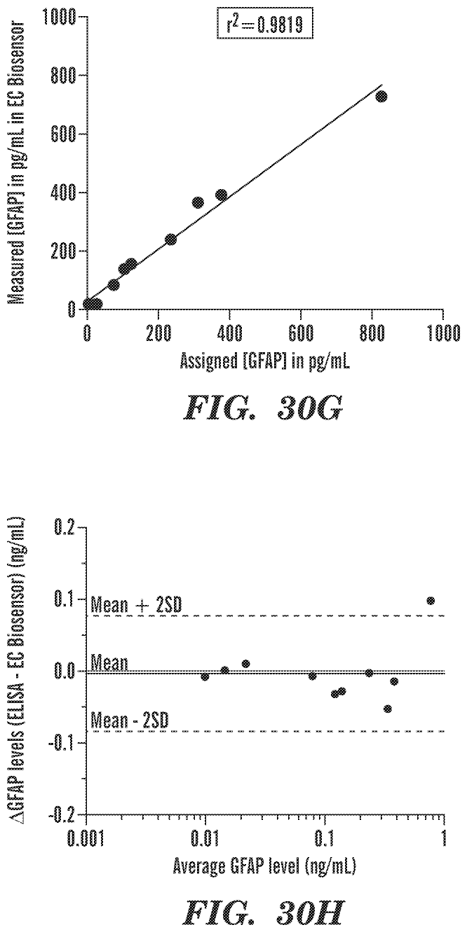

FIG. 30A-30H illustrates microfluidic integration and clinical validation of the assay. FIG. 30A is a picture of a microfluidic device where six EC-Biosensors can be placed to run the assay in parallel. FIG. 30B is a 3D schematic of the microfluidic channels and their interface with the EC-Biosensor. FIG. 30C shows a calibration curve for the assay of cTnITC performed on the microfluidic platform with reduced assay time using spiked plasma samples. FIG. 30D shows a calibration curve for the assay of GFAP performed on the microfluidic platform using spiked plasma samples. FIGS. 30E and 30F are plot showing validation of the EC Biosensor using standard 96 well assay for the detection of cTnITC (30E) and GFAP (30G) with n=3 for each clinical sample. Bland-Altman plot for validation of EC Biosensor using clinical sample for cTnITC is shown by FIG. 30F and for GFAP is shown by FIG. 30H. Scale bar size: 5 mm. Error bars represent the s.d. of the mean, n=3.

Under the optimized assay conditions of cTnI, current density from adsorbed TMB was directly proportional to the concentration of cTnI (0.01-10 ng/mL) (FIG. 24B and FIG. 10). The limit of detection (LOD) was found to be 9 pg/mL (LoB=Mean blank+1.645 (SD blank). Likewise, LoD=LoB+1.645 (SD low concentration sample). Assay conditions were also optimized to run the assay of cTnI in spiked plasma in a 96-well ELISA plate with LOD of 67 pg/mL Table 4. Troponins released into the bloodstream are mainly non-covalent ternary complex, cTnI-T-C(ITC complex), and binary complex cTnI-C(IC complex) with some free form of cTnI to some extent. Thus, EC-Biosensor and a 96 well plate was used for the detection of cTnITC complex. FIG. 24E shows the calibration curve of cTnITC with LOD of 3, 24, and 17 pg/mL for EC-Biosensor, 96 well plasma, and buffer, respectively. LOD obtained is better than the 99th percentile cutoff value (28 pg/mL) in a healthy population used for cardiac troponin assay.

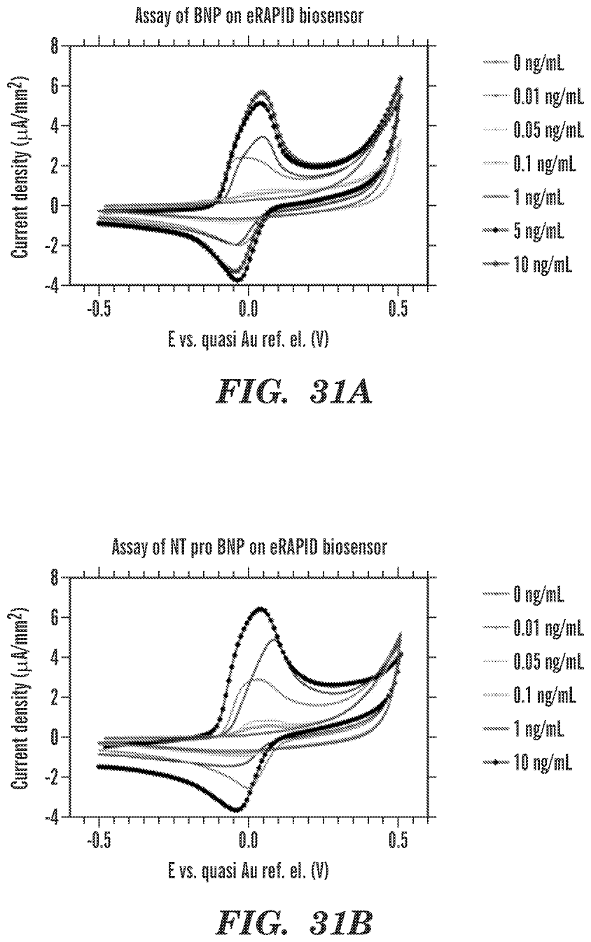

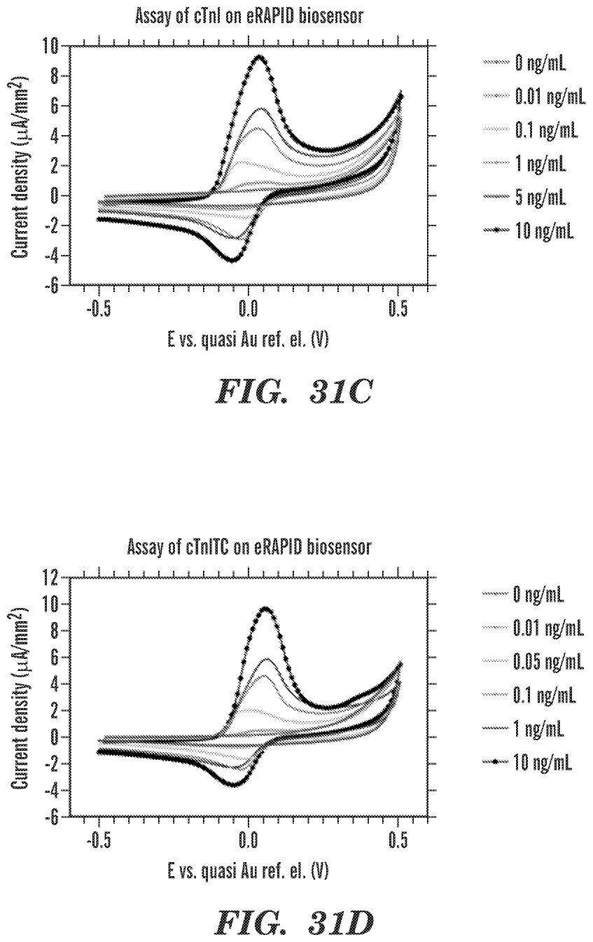

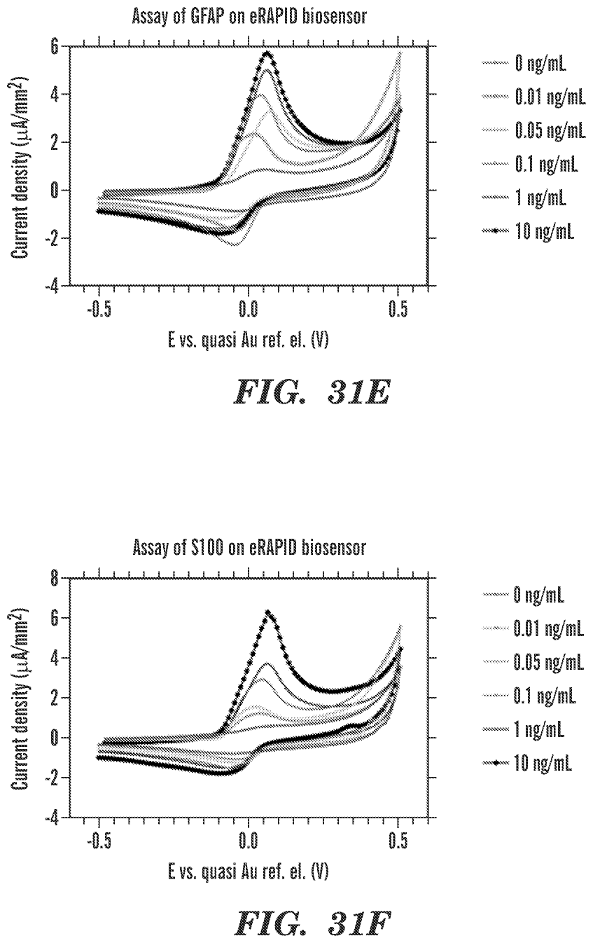

FIG. 31A-31F illustrates an assay of different biomarkers on EC-Biosensor. TMB oxidation and reduction peaks obtained with a CV on EC Biosensors with antifouling coating for detection of different concentrations of biomarkers of Myocardial infarction and Traumatic Brain Injury. FIG. 31A shows BNP, FIG. 31B shows NT-proBNP, FIG. 31C shows cTnI, FIG. 31D shows cTnITC, FIG. 31E shows GFAP, and FIG. 31F shows S100b.

TABLE 4
Sensitivity and Kd values for detection of biomarkers of MI and
TBI in EC-Biosensor with antifouling coating and 96 well assay.

| | | | | | | | | |
|---|---|---|---|---|---|---|---|---|
| | EC Biosensor | Plasma | 1 h 36 min | 15 | μL | 0.856 | 24 | pg/ml |
| | (2-step) | | | | | | | |
| | 96 well | Plasma | 2 h 40 min | 100 | μL | 14.9 | 67 | pg/mL |
| | 96 well | Buffer | 2 h 40 min | 100 | μL | 11.92 | 9 | pg/mL |
| BNP | EC Biosensor | Plasma | 37 min | 15 | μL | 0.19 | 26 | pg/mL |
| | 96 well | Plasma | 2 h 40 min | 100 | μL | ~187331 | 2783 | pg/mL |
| | 96 well | Buffer | 2 h 40 min | 100 | μL | 0.3255 | 24 | pg/mL |
| NT-proBNP | EC Biosensor | Plasma | 37 min | 15 | μL | 1.323 | 4 | pg/mL |
| | EC Biosensor | Plasma | 1 h 36 min | 15 | μL | 0.929 | 3 | pg/ml |
| | (2-step) | | | | | | | |
| | 96 well | Plasma | 2 h 40 min | 100 | μL | 3.509 | 65 | pg/mL |
| | 96 well | Buffer | 2 h 40 min | 100 | μL | 2.99 | 54 | pg/mL |
| cTnITC | EC Biosensor | Plasma | 37 min | 15 | μL | 0.1663 | 3 | pg/mL |
| | EC Biosensor | Plasma | 15 min | 40 | μL | 0.083 | 14 | pg/mL |
| | (microfluidics) | | | | | | | |
| | EC Biosensor | Whole | 37 min | 15 | μL | 0.0837 | 22 | pg/mL |
| | | blood | | | | | | |
| | 96 well | Plasma | 2 h 40 min | 100 | μL | 1.19 | 24 | pg/mL |
| | 96 well | Buffer | 2 h 40 min | 100 | μL | 1.439 | 17 | pg/mL |
| GFAP | EC Biosensor | Plasma | 37 min | 15 | μL | 0.0688 | 5 | pg/mL |
| | EC Biosensor | Plasma | 15 min | 40 | μL | — | 27 | pg/mL |
| | (microfluidics) | | | | | | | |
| | EC Biosensor | Whole | 37 min | 15 | μL | 0.0642 | 2 | pg/mL |
| | | blood | | | | | | |
| | 96 well | Plasma | 2 h 40 min | 100 | μL | 83.59 | 59 | pg/mL |
| | 96 well | Buffer | 2 h 40 min | 100 | μL | 2.878 | 15 | pg/mL |
| S100b | EC Biosensor | Plasma | 37 min | 15 | μL | 6.105 | 1 | pg/mL |
| | EC Biosensor | Whole | 37 min | 15 | μL | 0.3403 | 13 | pg/mL |
| | | Blood | | | | | | |
| | 96 well | Plasma | 2 h 40 min | 100 | μL | ~1.0e+016 | 957 | pg/mL |
| | 96 well | Buffer | 2 h 40 min | 100 | μL | 198.8 | 432 | pg/mL |

BNP (which degrade in circulation rapidly) and NT-proBNP show variable levels in circulation according to the clinical condition and timing of measurement for MI, which necessitates a rapid and sensitive POC device. Using optimized assay conditions for BNP, a sensitivity of 26 pg/mL was obtained with EC-Biosensor, which was almost 1000× better than 96 well plate (2.7 ng/mL) (FIG. 24C). Likewise, FIG. 24D shows the assay of NT-proBNP with LOD of 4 pg/mL, 65 pg/mL, & 54 pg/mL for spiked plasma samples in EC-Biosensor, 96 well plasma, and buffer, respectively. The sensitivity for EC-Biosensor was far better than the clinical cut-off values for BNP (35 pg/mL) and NT-proBNP (125 pg/mL) that have high negative predictive values (0.94-0.98) and can be used for ruling-out HF according to ESC guidelines.

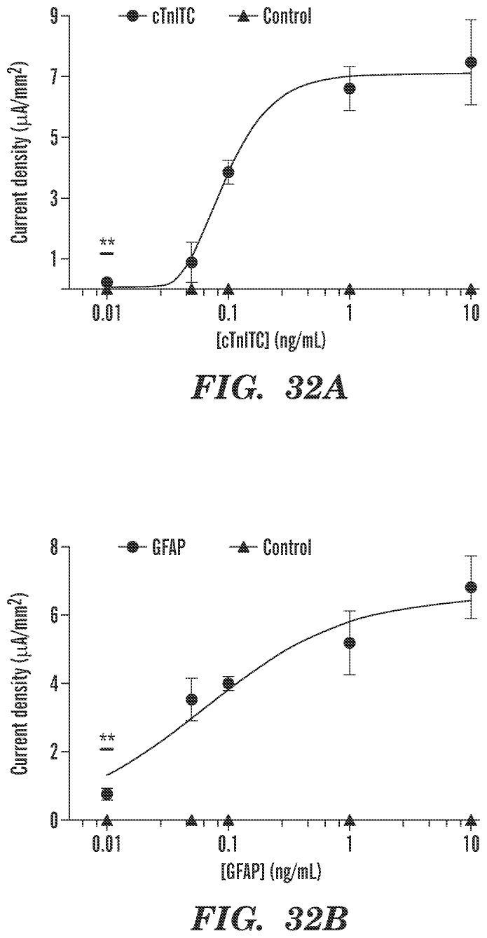

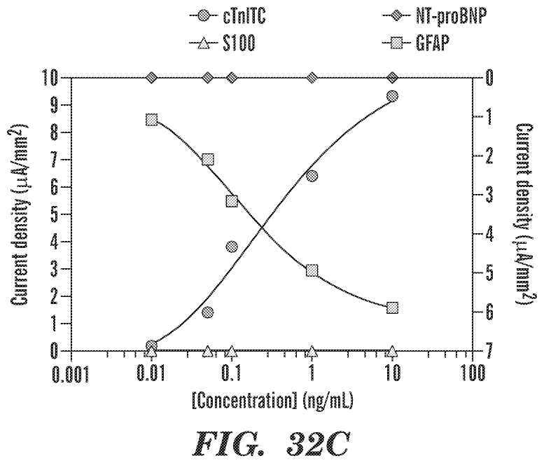

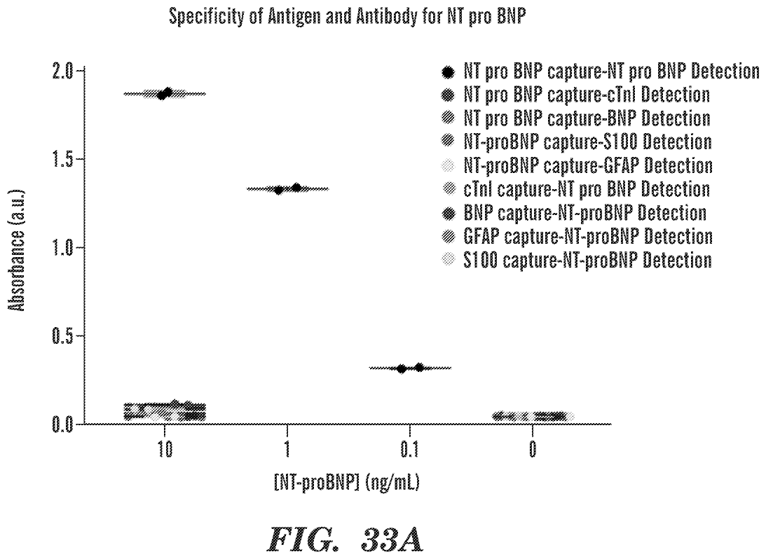

Changes in the expression of S-100b and GFAP markers have been shown to correlate with injury magnitude, survivability, and neurologic outcome of TBI. As shown in FIG. 24F, the oxidation current density increased linearly with increasing concentration of GFAP in the range of 10 pg/mL to 10 ng/mL. The LOD was found to be 5 pg/mL for EC-Biosensor while spiked plasma and buffer in 96 well gave LOD of 59 and 15 pg/mL, respectively. Likewise, as shown in FIG. 24G, LOD for S100b was found to be 1 pg/mL in EC-Biosensor while 96 well assay for S100b in spiked plasma and buffer had LOD of 957 and 432 pg/mL, respectively. LOD of both GFAP and S100b were better than the clinical cut-off value of 230 pg/mL for GFAP and 470 pg/mL for S100b. Calibration curves for the assay of cTnITC and GFAP were also performed with unprocessed whole blood with LOD of 22 and 2 pg/ml, respectively, which shows the broader application of the EC-Biosensor and its efficacy in POC settings. FIG. 32A-32C show calibration curves of different biomarkers run on the EC-Biosensor using unprocessed whole blood. Y-axis shows the current density for different concentrations of biomarkers run on EC Biosensors. FIG. 32A depicts a calibration curve of cTnITC; FIG. 32B depicts a calibration curve of GFAP, and FIG. 32C depicts multiplex detection of cTnITC and GFAP on EC Biosensor with four different capture antibodies on each electrode (anti-cTnITC, anti-S-100b, anti-GFAP, and anti-NT-proBNP). Error bars represent the s.d. of the mean, n=3. In 32A and 32B, significance was determined by unpaired t-test (^{ns}P>0.05; *P<0.05; **P<0.01; both two-tailed. All the experiments ran on the EC-Biosensor had one electrode modified with BSA instead of the capture antibody for the on-chip negative control. No current was observed, signifying that the nanocomposite coating had excellent antifouling properties, and the precipitation of TMB is highly localized without cross-contamination to the neighboring electrodes.

To reduce the overall assay time, enhance the analyte transport mechanism, and increase binding kinetics, a microfluidic device was developed with an integrated EC-Biosensor for parallel testing of six sensors (FIGS. 30A and 30B). LOD of 14 pg/mL and 27 pg/mL was obtained for cTnITC and GFAP, respectively, using microfluidic EC-Biosensors within 15 min (FIGS. 30C and 30D).

### Stability of Nanocomposite Material and Precipitated TMB.

Using NT-proBNP as a model, the rapid coating was compared with the previously published 24 h coating method. As seen in FIG. 28A there was no significant difference in signal between rapid and overnight coating, which shows that rapid coating with 45s is as efficient as 24 h coating. A full calibration curve of cTnITC was also compared on rapidly coated and 24 h coated sensors FIG. 28B. Although the current density is slightly higher at a higher concentration for 24 h coated EC-Biosensor at a lower concentration, they have a similar current density which shows that the coating time can be drastically reduced from 24 h to under a min by the rapid coating strategy with similar sensitivity. Next, the stability of precipitated TMB after the completion of the assay was studied. FIG. 28C shows no significant difference in current density between the EC-Biosensor stored for 24 h at room temperature after the assay to the fresh EC-Biosensors for detection of cTnITC over the whole calibration range. In addition, assay of GFAP at different concentrations was performed in four sets of EC-Biosensors in parallel and was measured on different days (1, 2, 4, and 8 days). As seen in FIG. 28D, no significant difference in signal was found between fresh EC-Biosensor and EC-Biosensor measured after storing in room temperature for up to 8 days, which shows that the precipitated TMB offers the flexibility of localized precipitation even under fluctuating condition and be reliably stored for at least 8 days after the completion of the assay.

### Multiplexed Electrochemical Detection of MI and TBI Markers.

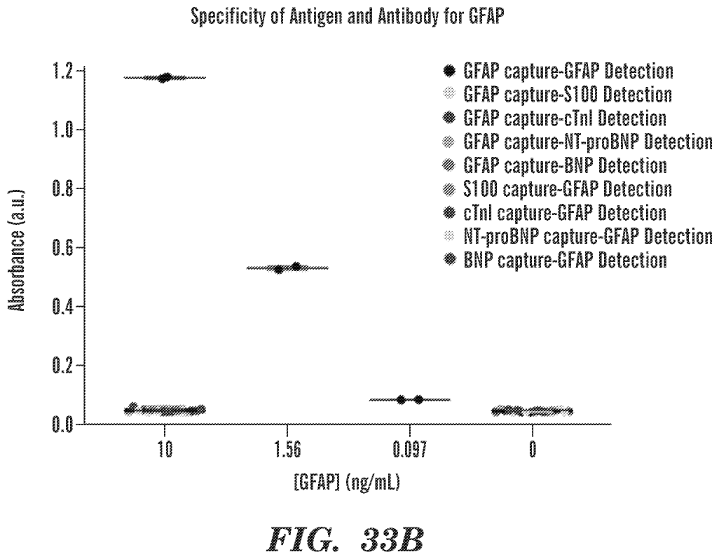

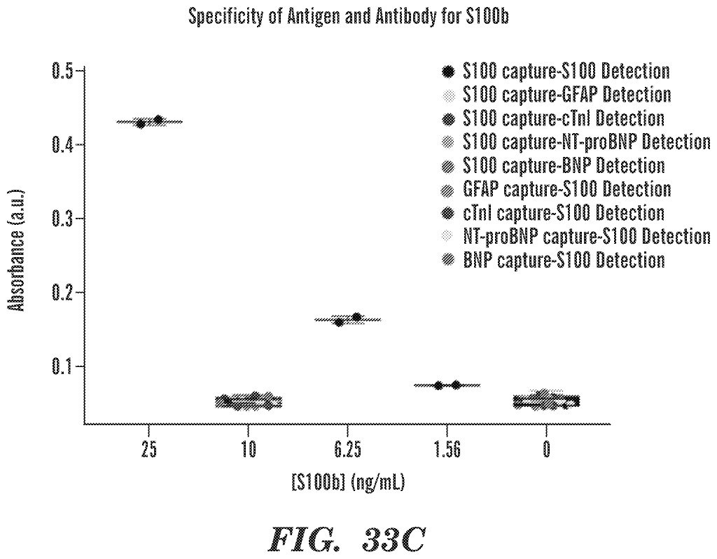

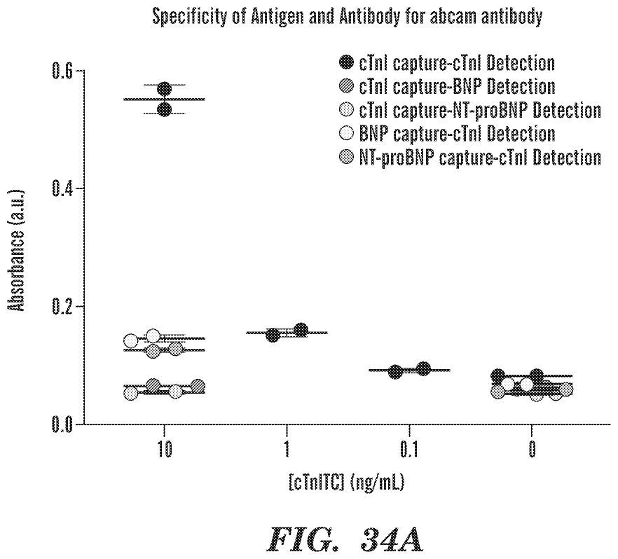

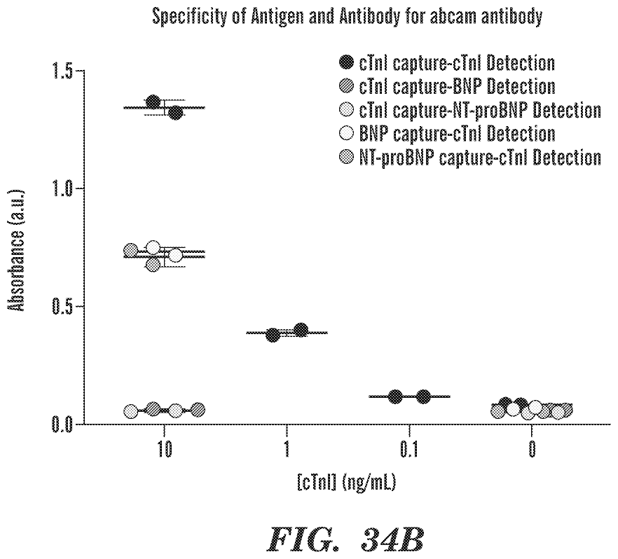

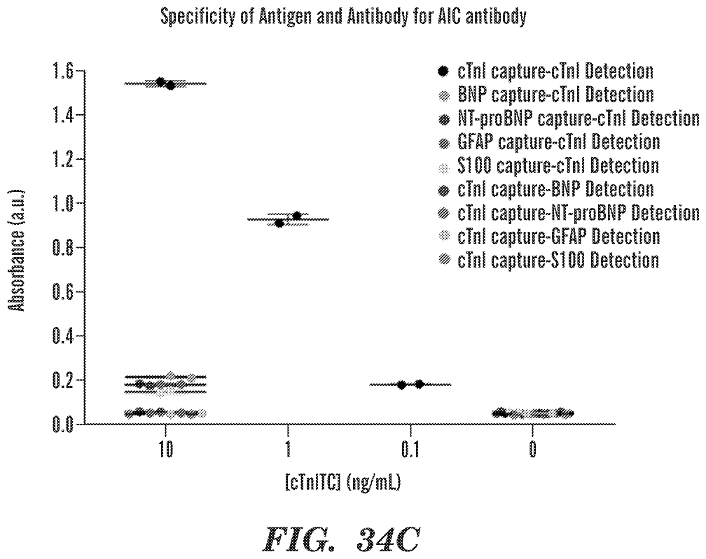

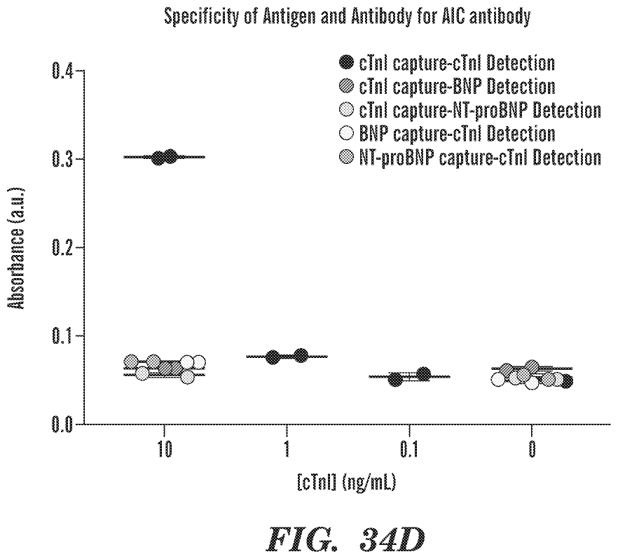

A thorough investigation of specificity and cross-reactivity of both MI and TBI biomarkers were tested against capture and detection antibodies of each of these markers. As shown in FIG. 29B, FIG. 33A-33C, and FIG. 34A-34D, BNP, NT-proBNP, GFAP, and S100b did not show any cross-reactivity with capture and detection antibody of other biomarkers. Abcam anti-cTnI antibody pair showed cross-reactivity with BNP and NT-proBNP capture antibody; thus, anti-cTnITC antibody pair which did not show any cross-reactivity with other antibodies were used for multiplexing experiments.

FIG. 33A-33C illustrate the specificity and cross-reactivity test for different biomarkers of MI and TBI done in 96 well plate. FIG. 33A shows the specificity and cross-reactivity of NT-proBNP antigen against different capture antibody (anti-cTnITC, anti-BNP, anti-GFAP, and anti-S-100b) and detection antibody (anti-cTnITC, anti-BNP, anti-GFAP, and anti-S-100b) along with specific detection with anti-NTpro BNP capture and detection antibody at different concentration of NT-proBNP. FIG. 33B shows the specificity and cross-reactivity of GFAP antigen against different capture antibody (anti-cTnITC, anti-BNP, anti-NT-proBNP, and anti-S-100b) and detection antibody (anti-cTnITC, anti-BNP, anti-NT-proBNP, and anti-S-100b) along with specific detection with GFAP capture and detection antibody at different concentration of GFAP. FIG. 33C shows the specificity and cross-reactivity of S-100b antigen against different capture antibodies (anti-cTnITC, anti-BNP, anti-NT-proBNP, and anti-GFAP) and detection antibody (anti-cTnITC, anti-BNP, anti-NT-proBNP, and anti-GFAP) along with specific detection with S-100b capture and detection antibody at different concentration of GFAP.

FIG. 34A-34D illustrates a specificity and cross-reactivity test for different Troponin antibody pair and antigen done in 96 well plate. FIG. 34A shows specificity and cross-reactivity of cTnITC antigen against different capture antibody (anti-NT-proBNP and anti-BNP) and detection antibodies (anti-NT-proBNP and anti-BNP) along with specific detection with anti-cTnITC capture and detection antibody from abcam at different concentration cTnITC. FIG. 34B shows specificity and cross-reactivity of cTnI antigen against different capture antibodies (anti-NT-proBNP and anti-BNP) and detection antibodies (anti-NT-proBNP and anti-BNP) along with specific detection with anti-cTnI capture and detection antibody from abcam at different concentration cTnI. FIG. 34C shows specificity and cross-reactivity of cTnITC antigen against different capture antibody (anti-NT-proBNP, anti-BNP, anti-GFAP, and anti-S-100) and detection antibody (anti-NT-proBNP, anti-BNP, anti-GFAP, and anti-S-100b) along with specific detection with anti-cTnITC capture and detection antibody from Advanced ImmunoChemical Inc at different concentration of cTnITC. FIG. 34D shows specificity and cross-reactivity of cTnI antigen against different capture antibodies (anti-NT-proBNP and anti-BNP) and detection antibodies (anti-NT-proBNP and anti-BNP) along with specific detection with anti-cTnI capture and detection antibody from Advanced ImmunoChemical Inc at different concentration of cTnI.

To further demonstrate that four biomarkers can be multiplexed and run in parallel within a single chip, each sensor's electrode was individually functionalized with specific capture antibodies (anti-cTnITC, anti-S100b, anti-NT-proBNP, and anti-GFAP) (FIG. 29A). Initially, a calibration curve of cTnITC was run on the multiplexed platform where an increase in the current density was observed, which was directly proportional to the concentration of cTnITC (FIG. 29C), while the rest of the electrodes showed no signal, which shows that there is no cross-reactivity. Subsequently, two calibration curves were run in parallel, cTnITC from lower to higher concentration and GFAP from higher to lower concentration (FIG. 29D), showing only the specific signal for each marker. A similar calibration curve was also performed in whole blood with similar results (FIG. 32C). Finally, (FIG. 29E) shows the calibration curve of all four biomarkers in parallel. Two calibration curves for cTnITC and S100b were run from lower to higher concentration, while the other two biomarkers, NT-proBNP and GFAP, were run from higher to lower concentration. Running calibration curves in the opposite direction ensures that non-specific signals from the high concentration of biomarkers (if any) will be observed at the other end with low concentration, which may be masked if all calibration curves are run in the same direction. As expected, the high specificity of antibody pair to the respective analyte was observed as no nonspecific signal was observed even at a very high concentration of other analytes.

To examine the storage condition of the coating solution, the nanocomposite was stored at 4° C. and ambient temperature for up to 15 weeks. After the storage of nanocomposite for different periods, sensors were rapidly coated with nanocomposite to perform the GFAP assay. 92-113% current response was maintained for the coating stored at 4° C. for 15 weeks, while 96-104% current response was maintained for the coating stored at room temperature for 12 weeks compared to fresh coating for the assay of GFAP (FIGS. 28E and 28F). Antifouling nanocomposite developed so far have been tested only under laboratory conditions and not been commercialized, so this storage stability of the nanocomposite has enormous potential for commercialization and manufacturing in reel-to-reel format as it can be easily prepared, stored, and rapidly coated to the EC-Biosensor for developing a marketable device with reliable functioning.

### Clinical Validation.

The developed EC-Biosensors were clinically validated with 22 patient plasma samples (12 for cTnITC and 10 for GFAP). The data obtained from the EC-Biosensor were compared to the data from conventional 96 well plate ELISA. The values obtained from both methods were plotted in a scatter plot (FIGS. 30E and 30G) where the linear regression analysis showed r 2 of 0.9848 for cTnITC and 0.9819 for GFAP, showing an excellent correlation between the EC-Biosensor and conventional assay. There also was excellent agreement between the two methods in the lower concentration region (below 1 ng/mL) when a Bland-Altman plot was used (FIGS. 30F and 30H). The mean line represents the bias of the EC sensor, and the Mean±2SD are limits of the agreement in 95% confidence interval. The bias of the cTnITC EC-Biosensor was 0.02 ng/mL with Mean+2SD and Mean −2SD value of 0.18 ng/mL and −0.13 ng/mL, respectively. Similarly, a bias of the GFAP EC-Biosensor was −0.003 ng/mL with Mean+2SD and Mean −2SD value of 0.07 ng/mL and −0.08 ng/mL, respectively.

### Discussion

POC EC-Biosensors suffer predominantly from surface fouling via biological samples and inconvenient mass manufacturing protocol. In addition, current research trends in the direction of experimental analysis in single-protein fouling solutions like BSA and lack studies in different real-world biological samples and thus do not trend towards commercial translation. The developed frugal EC-Biosensor coating process enables stable antifouling properties through a rapid and high-throughput process which could be easily translated into reel-to-reel mass manufacturing process. The coated gold electrodes retained more than 88% of current density for up to 9 weeks of incubation in unprocessed human plasma. Furthermore, nanocomposite coating could be stored at room temperature for at least 12 weeks with 96-104% current response demonstrating their long-term stability. Excellent anti-fouling properties shown by the coating in complex biological samples such as plasma, whole blood, saliva, and urine opens the door to combat future crises that may be similar to the current global coronavirus pandemic as the POC EC-Biosensor platforms could be easily manufactured. In addition, it could be operated with minimum training and provide highly sensitive and specific multiplexed detection and can be easily deployed into the field. The stability of precipitated TMB over a week provides the flexibility of shipping the sensor to the central laboratory from a POC setting.

### Materials and Methods

Anti fouling nanocomposite preparation: The BSA/prGOx/GA anti-fouling nanocomposite was prepared by mixing 8 mg/mL of pentaamine functionalized reduced graphene oxide (prGOX) (Millipore Sigma, no. 806579) with 5 mg/mL (IgG-Free, Protease-Free, Bovine Serum Albumin) (Jackson ImmunoResearch, no. 001-000-162) in 10 mM phosphate-buffered saline solution (PBS, pH 7.4) (Sigma Aldrich, USA, no. D8537). A similar method was used to prepare the coating with amine-functionalized reduced graphene oxide (Sigma Aldrich, USA, no. 805432). The solution was sonicated in a tip sonicator for 30 min using 1 s on/off cycles at 50% amplitude, 125 W and 20 kHz (Bransonic, CPX 3800) followed by heating (Labnet, no. D1200) at 105° C. for 5 min to denature the protein. The resulting opaque black mixture was centrifuged at 16.1 relative centrifugal force for 15 min to remove the excess aggregates. The semi-transparent nanocomposite supernatant solution was then mixed with 70% glutaraldehyde (Sigma Aldrich, USA, no. G7776) for crosslinking in the ratio of 70:2.

Cleaning of sensor: The gold electrode chips were custom fabricated using a standard photolithography process and purchased from Telic Company. Briefly, the gold wafer was deposited with 12-15 nm of chromium followed by 100 nm of gold over it. Inner electrodes were 0.448 mm in diameter (surface area of 0.1576 mm²) while reference electrodes diameter was 1.15 mm in diameter (surface area of 1.038 mm²). Before coating the chip with nanocomposite, chips were cleaned by sonicating in acetone (Sigma Aldrich, USA, no. 650501) for 10 min followed by isopropanol (Sigma Aldrich, USA, no. W292907) for another 10 min to remove the photoresist. To ensure that the surface of the chips is clean, the chips were then treated with oxygen plasma using a Zepto Diener plasma cleaner (Diener Electronics, Germany) at 0.5 mbar and 50% power for 2 min.

Development of coating strategy of anti fouling nanocomposite: To develop the best coating strategy of the anti-fouling nanocomposite to the gold electrodes of the chips; cleaned and plasma-treated chips were first kept over a hot plate for 2 min to let the temperature equilibrate to 85° C. 70 μL of anti-fouling nanocomposite as described above was then drop cast to each chip and incubated for 30s-5 minutes. The chips were then washed by dipping in PBS immediately or after cooling down for 10 min followed by washing at 400 rpm for 10 min. The chips were then exposed to 1M ethanolamine (Sigma Aldrich, USA, no. E9508) in PBS to quench the unreacted glutaraldehyde groups before electrochemical analysis. For optimization of BSA-glutaraldehyde ratio of the anti-fouling coating, nanocomposite solution was prepared with different concentrations of BSA ranging from 0.5 mg/mL to 10 mg/mL with 3 different ratios of BSA & GA (1:70, 2:70, and 4:70). The chips were coated with these solutions for 45s at 85° C. followed by washing and electrochemical analysis. To further develop the nanocomposite that can retain the maximum signal and have the best anti-fouling properties, nanocomposite was prepared with 5 mg/mL BSA with different concentration of prGOx (0.5, 1, 2, 4, 8, 10, 12, 15 mg/mL) with GA/prGOx/BSA of 2:70. Electrochemical analysis was done after quenching the reaction with 1M ethanolamine for 30 min, followed by 1 hr incubation in 1% BSA.

Rapid antifouling coating recipe combined with extensive assay development helped in significant improvement of the EC-Biosensor performance as demonstrated by the increased sensitivity and wide dynamic and linear range of the EC-Biosensor. The porous BSA backbone of the matrix prevents non-specific protein adsorption but allows diffusion of electroactive soluble species. The employment of prGOx, which is highly conductive, accelerated electron transfer and enhanced the transduction properties. Finally, developing the optimized conditions of each component of the sandwich assay led to enhancing the current response, resulting in highly sensitive immunoassays. EC-Biosensor also showed excellent correlation with reported values in clinical samples and thus has huge potential for commercial POC detection.

Electrochemical Characterization of Anti-Fouling Nanocomposite.

All the electrochemical characterization and assays were carried out on the chip containing four working gold electrodes, a common pseudo-reference gold electrode, and a common gold counter-electrode connected to a potentiostat (Autolab PGSTAT128N, Metrohm; VSP, Bio-Logic) through an in-house built connector device. The conductive anti-fouling nanocomposite was electrochemically characterized by cyclic voltammograms (CV) in a redox aqueous solution of PBS containing 5 mM [Fe(CN)₆]^{3-/4-} at a scan rate of 200 mV/s between −0.5 to 0.5V. For anti-fouling properties chips were incubated with plasma (Innovative Research, USA, no. IPLASCOV2P100UL), whole blood (Innovative Research, USA, no. IWB1NAH10ML), urine (drug/alcohol-free, LEE Biosolutions, USA, no. 991-03-DF-50), and saliva (avantor, USA, no. 102768-970) for different time before electrochemical measurement. In addition, to characterize the reversibility of the system, bare gold electrodes, gold electrodes with BSA, gold electrode with BSA and prGOx, gold electrodes with BSA and GA, gold electrodes covered with nanocomposite were analyzed by CV in PBS containing 5 mM [Fe(CN)₆]^{3-/4-} at 200 mV/s between −0.5 to 0.5V. Finally, bare gold electrodes and gold electrodes covered with nanocomposite were analyzed by CV in PBS containing 5 mM [Fe(CN)⁶]^{3-/4-} by increasing the scan rate from 10 to 1000 mV/s between −0.5 to 0.5V.

Spectroscopic/Microscopic study of gold electrode/anti-fouling nanocomposite: The nanocomposites along with intermediate components were characterized by UV spectroscopy (Nanodrop 2000C, Thermo Scientific) after the addition of every component, to elucidate changes in the absorbance bands of the peptide backbone or the aromatic rings due to the crosslinking of BSA and GOx with GA. Topographic characterization for the nanocomposite and other coatings over the sensor was carried out by SEM (Verios G4 XHR, Thermo Fisher Scientific). The samples were first coated with a thin layer of approximately 6 nm Cr by sputtering (K755X EM Technologies LTD.). Imaging was carried out by detection of secondary electrons using an in-lens detector at an accelerating voltage of 5 kV. For TEM characterization, samples were prepared using the in-situ FIB lift-out technique on an FEI Helios 650 Dual Beam FIB/SEM. The samples were capped with sputtered Ir, protective carbon and e-Pt/I-Pt before milling. To obtain better contrast of the coating one set of TEM was done without Ir. The TEM lamella thickness was −100 nm. The samples were imaged with an FEI Tecnai Talos FEG/TEM operated at 200 kV in bright-field (BF) TEM mode and high-resolution (HR) TEM mode. Similarly, AFM images were collected using a Dimension Icon AFM instrument (Bruker, Santa Barbara, California, USA). The instrument was calibrated against a NIST traceable standard. Soft Tapping Mode was used as analysis mode with OTESPA-R3 (Bruker) as AFM probe. 1s t order flattening was used for data post-processing. One 1 μm×1 μm area was imaged near the center of each sample. AFM 2D and 3D height images were obtained along with the roughness measurements of gold and gold/coating. The topography differences of these images are presented in colors where the brown is low and the white is high. The z ranges are noted on the vertical scale bar on the right side of the images. RMS (R q) is the standard deviation of the Z values (or RMS roughness) in the image. It was calculated according to the formula: R_{q}=Ö{S(Zᵢ−Z_{avg})²/N}, where Z_{avg} is the average Z value within the image; Zᵢ is the current value of Z, and; N is the number of points in the image. Mean roughness (Rₐ) is the mean value of the surface relative to the Center Plane and was calculated using the formula: Rₐ=[1/(LₓL_{y})]ò₀^{Ly}ò₀^{Lx}{f(x,y)}dxdy, where f(x,y) is the surface relative to the Center Plane, and Lₓ and L_{y} are the dimensions of the surface. Max height (Rₘₐₓ) is the difference in height between the highest and lowest points of the surface relative to the Mean Plane. Surface area is the area of the 3-dimensional surface of the imaged area. It was calculated by taking the sum of the areas of the triangles formed by 3 adjacent data points throughout the image. Surface area diff is the amount that the Surface area is over the imaged area. It was expressed as a percentage and is calculated according to the formula: Surface area diff=100[(Surface area/S₁²)−1], Where Sₗ is the length (and width) of the scanned area minus any areas excluded by stopbands. X-ray Photoelectron Spectroscopy (XPS) was used to determine semi-quantitative atomic composition and chemistry using PHI Quantum 2000 instrument. Monochromated Alkaa 1486.6 eV was used as X-ray source with an acceptance angle of ±23°, take-off angle of 45°, and analysis area of 1400 μm×300 μm. TOF-SIMS was performed using IONTOF TOF-SIMS 5 instrument and data were obtained using a liquid metal ion gun (LMIG) primary ion source. Both surface spectrum and depth profile were acquired from the sample. Ar-cluster was used as ion source with Ion beam potential of 2.5 keV on an area of 500 μm×500 μm for sputtering. Likewise, Bi₃⁺ was used as ion source with Ion beam potential of 30 keV on an area of 200 μm×200 μm for analysis. The data are presented as mass spectra which are displayed as the number of secondary ions detected (Y-axis) versus the mass-to-charge (m/z) ratio of the ions (X-axis). The ion counts are shown on linear intensity scales, and probable empirical formulae for several peaks are identified in the figures.

Conjugation of Capture Antibodies: After coating the electrodes with nanocomposite coating, chips were washed in PBS by agitation (400 rpm) for 10 min and dried with a slide spinner (Millipore Sigma, no. 674664). 1-ethyl-3-(3-dimethylaminopropyl) carbodiimide hydrochloride (EDC, Thermo Fisher Scientific, no. 22980) and N-hydroxysuccinimide (NHS, Sigma Aldrich, no. 130672) were dissolved in 50 mM IVIES (2-(N-morpholino)ethanesulfonic acid) Buffer (pH 6.2) at 400 mM and 200 mM, respectively and deposited on nanocomposite covered gold chips for 30 min at room temperature in dark. After surface activation with EDC/NHS, chips were quickly rinsed with Milli Q water and dried with compressed air. Thereafter, spotting of capture antibody on top of the working electrode area using Xtend capillary microarray Pin (LabNEXT, no. 007-350) was performed. The fourth working electrode was always spotted with 5 mg/mL BSA as a negative control unless mentioned otherwise. The spotted chips were stored overnight at 4° C. in a humidity chamber. After conjugation, chips were washed with PBS and quenched with 15 μL of 1M ethanolamine (SigmaAldrich, no. E9508) for 30 min and blocked with 10 μL of 2.5% BSA in PBS for 1 hour.

Optimization of Assay Conditions and Biomarker Detection Using Two-Step Assay

Conjugation of biotin to detection antibody was done using Biotin Conjugation Kit (Fast, Type A)—LIGHTNING-LINK® (abcam, USA, no. ab201795) using manufacturer's protocol. Briefly, detection antibody was diluted to 1 mg/mL in PBS followed by addition of 1 μl of modifier/10 μl of antibody and mixed to the linker for 30 min and finally, the reaction was quenched by adding 1 μl of quencher/10 μL of solution. The biotin-conjugated antibody was ready to be used after 5 min. For optimization of detection antibody for two-step assay of cTnI, 1 mg/mL of anti-cTnI capture antibody (abcam, no. ab243982) was spotted to three working electrodes and BSA on fourth as a negative control. 1 ng/mL and 0 ng/ml of cTnI (Medix Biochemica, no. 610102) was spiked into plasma (Innovative Research, IPLASNAHUNIT) and 15 μL of the sample was incubated with agitation at 400 rpm for 1 hr followed by washing with PBST (PBS with 0.05% Tween 20 (Sigma Aldrich, no. P9416)). 10 μL of different concentrations (0.1, 1, 2, and 5 μg/mL) of the biotinylated anti-cTnI detection antibody (abcam, no. ab243982) prepared in 0.1% BSA in PBST were added for 15 min followed by washing and addition of 10 μL of Poly-HRP-Streptavidin (Thermo Fisher Scientific, no. N200) diluted to 1 μg/mL in 0.1% BSA in PBST. Chips were washed after incubating in Poly-HRP-Streptavidin for 5 min. 10 μL of precipitating 3,3′,5,5′-Tetramethylbenzidine (TMB, Sigma-Aldrich, USA, no. T9455) was added to chips and incubated for 1 min before washing. Finally, measurement was performed in PBST using a potentiostat by a CV with a scan rate of 1 V/s between −0.5 and 0.5 V vs on-chip integrated gold quasi reference electrode. Peak height was calculated using Nova 1.11 software for data analysis. All the detection antibodies and Poly-HRP-Streptavidin in the following experiments were prepared in 0.1% BSA in PBST unless mentioned otherwise.

For assay of two-step cTnI, chips were spotted with anti-cTnI capture antibody followed by addition of plasma sample spiked with cTnI (0.1, 1, 5, and 10 ng/mL) for 1 hr. Optimized detection antibody concentration of 1 μg/mL was added and incubated for 15 min followed by the addition of 1 pg/mL of Poly-HRP-Streptavidin. Finally, precipitating TMB was added for 1 min before measuring the CV. Likewise, for the assay of two-step NT-proBNP, chips were spotted with anti-NT-proBNP capture antibody (Medix Biochemica, no. 100521) followed by the addition of plasma sample spiked with NT-proBNP (Medix Biochemica, no. 610090) (0.01, 1, and 10 ng/mL) for 1 hr. 1 μg/mL of biotinylated anti-NT-proBNP detection antibody (Medix Biochemica, no. 100712) was added and incubated for 15 min followed by addition of 1 μg/mL of Poly-HRP-Streptavidin. Finally, precipitating TMB was added for 1 min before measuring the CV.

Optimization of Assay conditions using a single-step assay: For optimization of single-step capture antibody for cTnI, different concentrations of capture antibody (50, 100, 500, and 1000 ng/mL) were spotted on the working electrodes. Three different concentrations of cTnI (0, 0.1, and 10 ng/mL) were mixed with biotinylated anti cTnI detection antibody in the ratio of 9:1 and 15 μL was added to each chip. Chips were washed after 30 min followed by the addition of Poly-HRP-Streptavidin for 5 min and TMB for 1 min before reading. Detection antibody for cTnI was optimized using the optimum concentration of the capture antibody. Briefly, chips were spotted with 500 ng/mL of anti-cTnI capture antibody. Three different concentrations of cTnI (0, 0.1, and ng/mL) was mixed with different concentrations of the biotinylated anti-cTnI detection antibody (final concentration of 1, 2, 3, 5, and 8 μg/mL) in the ratio of 9:1 and 15 μL was added to each chip. Chips were washed after 30 min followed by the addition of Poly-HRP-Streptavidin for 5 min and TMB for 1 min before reading. For optimization of Poly-HRP-Streptavidin chips were spotted with 500 ng/mL of anti-cTnI capture antibody. Three different concentrations of cTnI (0, 0.1, and 10 ng/mL) was mixed with 5 μg/mL biotinylated anti cTnI detection antibody. Chips were washed after 30 min followed by the addition of different concentrations of Poly-HRP-Streptavidin (1, 2, 3, 5, and 8 μg/mL) for 5 min. After washing the chips, TMB was incubated for 1 min followed by washing before reading. Finally, to complete the assay optimization for cTnI, precipitating TMB incubation time was optimized by spotting the chips with 500 ng/mL of anti-cTnI capture antibody. Different concentrations of cTnI (0, 0.01, 0.05, 0.1, 0.5, 1, and 10) were mixed with 5 μg/mL biotinylated anti-cTnI detection antibody and incubated on the chip for 30 min. 5 ug/mL of Poly-HRP-Streptavidin was added for 5 min followed by washing. One set of chips were incubated for 1 min with precipitating TMB while the other set was incubated for 5 min before washing and reading the chips. For all the subsequent experiments optimized conditions of 500 ng/mL of capture antibody, 5 μg/mL of Poly-HRP-Streptavidin, and 2 min of TMB precipitation time were used and only the concentration of detection antibody was optimized.

For optimization of detection antibody for BNP, chips were spotted with 500 ug/mL of anti-BNP capture antibody (HyTest, no. 50E1cc). Three different concentrations (0, 0.1, and 10 ng/mL) of BNP-32 (Bachem, no. 4095916) was mixed with different concentrations (final concentration of 3, 6, 9, 12, and 15 pg/mL) of the biotinylated anti-BNP detection antibody (HyTest, no. 24C5cc) in the ratio of 9:1 and 15 μL was added to each chip. Chips were washed after 30 min followed by the addition of Poly-HRP-Streptavidin for 5 min and TMB for 2 min before reading. Likewise, for optimization of detection antibody for NT-proBNP, chips were spotted with 500 ug/mL of anti-NT-proBNP capture antibody. Three different concentrations (0, and 10 ng/mL) of NT-proBNP were mixed with different concentrations (1, 3, 6, and 9 ng/mL) of the biotinylated anti-NT-proBNP detection antibody in the ratio of 9:1 and 15 μL was added to each chip. Chips were washed after 30 min followed by the addition of Poly-HRP-Streptavidin for min and TMB for 2 min before reading. Similarly, for optimization of detection antibody for cTnITC, chips were spotted with 500 ug/mL of anti-cTnI capture antibody (Advanced ImmunoChemical, no. 2-TIC-rc). Three different concentrations (0, 0.01, and 1 ng/mL) of cTnITC complex (HyTest, no. 8T62) was mixed with different concentrations (1, 3, 6, and 9 μg/mL) of biotinylated anti-cTnITC detection antibody (Advanced ImmunoChemical, USA no. 2-TC) in the ratio of 9:1 and 15 μL was added to each chip. Chips were washed after 30 min followed by addition of Poly-HRP-Streptavidin for 5 min and TMB for 2 min before reading.

Detection of Biomarkers using the EC-Biosensor: Detection of all the biomarkers was done on the chip using the optimized conditions. Three working electrodes were spotted with a capture antibody concentration of 500 pg/mL diluted in PBS. Anti-GFAP capture antibody (HyTest, no. GFAP83cc) and anti-S100b capture antibody (HyTest, no. 8B10cc) were used for the assay of GFAP (HyTest, no. 8G45) and S100b (HyTest, no. 8S9h), respectively. Different concentrations ranging from 10 pg/mL to 10 ng/mL were mixed with optimized detection antibody concentration of each biomarker (5 pg/mL for cTnI, 15 pg/mL for BNP, 6 μg/mL for NT-proBNP, and 6 pg/mL for cTnITC). 2 μg/mL of anti-GFAB detection antibody (HyTest, no. GFAP81cc) and anti S-100 detection antibody (HyTest, no. 6G1 cc) were used for the assay of GFAP and S100b, respectively. After incubating 15 μL of sample-detection antibody mixture for 30 min, chips were washed, and 5 μg/mL of Poly-HRP-Streptavidin was added for 5 min. Finally, precipitating TMB was incubated for 2 min before washing and reading the chips. Clinical sample validation was performed using the same optimized conditions for cTnITC and GFAP.

Detection of Biomarkers using 96 well colorimetric assay: For 96 well assay of the biomarkers, 100 μL of 1 μg/mL capture antibody prepared in carbonate-bicarbonate buffer at pH 9.2 was added to NUNC™ MAXISORP™ ELISA plates (BioLegend, no. 423501) and incubated overnight at 4° C. The plates were washed 3 times with 200 μL of PBST followed by the addition of 200 μL of 2.5% BSA for 1 h. After washing the plates 100 μL of different concentrations of the biomarkers (1% buffer/plasma) were added and incubated at 400 rpm for 1 h. After washing the plates biotinylated detection antibody (0.5 pg/mL for cTnI, NT-proBNP, cTnITC, and GFAP and 1 pg/mL for BNP and S100b) was added for 1 h before washing and adding 100 μL of Streptavidin-HRP (1:200 dilution in 0.1% BSA). The plate was washed and 150 μL of Turbo™B (Thermo Scientific, no. 34022) was added for 20 min followed by the addition of 150 μL of Stop solution to stop the reaction. The plate was immediately read using a microplate reader at 450 nm. For Clinical samples of cTnITC, samples were diluted 1:1 in 1% BSA. For Clinical Samples of GFAP, a magnetic bead-based assay from ThermoFisher Scientific was used following the protocol mentioned in the user guide, and measurement was done using Bio-Plex 3D Suspension Array System. Clinical samples received from Discovery Life Sciences in dry ice were aliquoted and stored at −80° C. until further use. All samples were collected under the approval of the Institutional Review Board for Harvard Human Research Protection Program (IRB21-0024).

Specificity of Antigen and Antibody for MI and TBI using 96 well colorimetric assay: Specificity of Antigen and Antibody was performed in 96 well ELISA plates. For each biomarker, four different concentrations were run with specific antibody pair to observe the signal for specific binding. To see if there is any non-specific binding of antigen to capture antibody of other biomarkers, all the non-specific capture antibodies were coated to the plates followed by the addition of high concentration analyte (10 ng/mL) and negative control (0 ng/ml) and detection antibody for the analyte. To test non-specific binding between the antigen and detection antibody of other biomarkers, specific capture antibody was coated to the plate and high concentration and negative control were added to the plate followed by non-specific detection antibody. All the assays were performed in buffer (1% BSA in PBS). For BNP specificity test, four concentrations of BNP (0, 0.1, 1, and 10 ng/mL) were tested with specific antibody pair for BNP. For non-specific capture antibody-antigen binding test, four different capture antibodies (anti-NT-proBNP, anti-cTnI, anti-GFAP, and anti-S100b) were coated at 1 μg/mL followed by addition of either 10 ng/mL or 0 ng/mL of BNP. After washing anti-BNP detection antibody was added followed by Streptavidin-HRP and TMB. Similarly, for non-specific antigen-detection antibody binding test, BNP capture antibody followed by 0 or 10 ng/mL of BNP was added. After washing, non-specific biotinylated detection antibodies (anti-NT-proBNP, anti-cTnI, anti-GFAP, and anti-S100b) were added followed by Streptavidin-HRP and TMB. Likewise, specificity test for other biomarkers including NT-proBNP, GFAP, and S100b was done similarly with specific and non-specific antigen-antibody pair. Likewise, for cTnI and cTnITC specificity test was performed with both abcam antibody pair (specific to cTnI) and Advanced ImmunoChemical antibody pair (specific to cTnITC).

Multiplexed detection of MI & TBI biomarkers using the EC-Biosensor: For Multiplexed detection of cTnITC, four different capture antibodies (anti-cTnITC, anti-S100b, anti-NT-proBNP, and anti-GFAP) was spotted on four different electrodes of the chip at 500 pg/mL. Increasing concentration of cTnITC (0, 0.01, 0.05, 0.1, 1, and 10 ng/mL) were mixed with optimum concentration of all the four biotinylated detection antibody. Chips were washed and Poly-HRP-Streptavidin was added for 5 min followed by TMB for 2 min before washing and reading the chips. In the next experiment for parallel detection of cTnITC and GFAP all capture antibodies were spotted as earlier. Increasing concentration of cTnITC and decreasing concentration of GFAP (0.01 cTnITC+10 GFAP; 0.05 cTnITC+1 GFAP; 0.1 cTnITC+0.1 GFAP; 1 cTnITC+0.05 GFAP; 10 cTnITC+0.01 GFAP) was mixed with all the four biotinylated detection antibody and incubated for 30 min. Likewise for parallel detection of all four biomarkers, all capture antibodies were spotted as earlier. Increasing concentration of cTnITC and S100b and decreasing concentration of GFAP and NT-proBNP (0.01 cTnITC & S100b+10 GFAP & NT-proBNP; 0.05 cTnITC & S100b+1 GFAP & NT-proBNP; 0.1 cTnITC & S100b+0.1 GFAP & NT-proBNP; 1 cTnITC & S100b+0.05 GFAP & NT-proBNP; 10 cTnITC & S100b+0.01 GFAP & NT-proBNP) was mixed with all four biotinylated detection antibody and incubated for 30 min. Chips were washed and Poly-HRP-Streptavidin was added for 5 min followed by TMB for 2 min before washing and reading the chips.

Rapid electrochemical assay with microfluidic integration of the EC-Biosensor: The microfluidic chip has several inlet and outlet ports and is covered with a special arrangement of cover and adhesive tapes to attach the sensor on top of the microfluidic chip. These tapes also define the flow channel between the sensor and the microfluidic chip (FIGS. 30A and 30B). The microfluidic chip was fabricated via 3D printing (Tiger APEX pro XHD 3D printer, Tiger 3D & Romanoff Int. Amityville, NY, USA). A clear photopolymer resin with low viscosity was used to fabricate the microfluidic chip (Tiger3D Clear 78-5011-L, Tiger 3D & Romanoff Int. Amityville, NY, USA). The following printing parameters were used: Layer thickness: 20 μm, Exposure time per layer: 2 s, Base exposure time: 10 s, Number of transitioning layers from base to normal exposure time: 4 layers, and UV wavelength: 405 nm. For post-processing, chips were cleaned in an isopropanol bath and cured under UV for 5 min (Form cure FH-CU-01, Formlabs Inc., Sommerville, MA, USA). A microfluidic cover tape was used to seal the chips (3M™ Microfluidic Diagnostic Tape 9795R, 3M Medical specialties, St. Paul, MN, USA). A double-sided adhesive tape was used to bond the sensor to the microfluidic chip and to pattern the sidewalls of the microfluidic channel sandwiched between them (ARSEAL™90880 Polypropylene Double-Sided Adhesive Tape, Adhesive research Inc., Glen Rock, PA, USA). The flow was initiated by peristaltic microfluidic pumps (RP-QII, Tagasako, Nagoya, Japan) driven by H-bridge brushed motor drivers (Pololu, Las Vegas, NV, USA). The pumps were controlled by a microcontroller (Arduino Uno, Arduino, Turin, Italy) via pulse density modulation to allow for high flow resolution at low pump speeds. The pump system was commanded through an LCD/keypad interface (Adafruit, New York, NY, USA). To perform an assay on the microfluidic platform, the sample mixed with detection antibody (40 μl) was added to inlet wells at the flow rate of 5 μl/min for 8 min. An optimized condition of detection antibody as discussed earlier was used. 25 μl of strep-HRP was then added at 5 μl/min for 5 min. Finally, 20 μl of TMB was added at 20 μl/min for 1 min and incubated under static incubation for 2 min. PBST wash was done after each step at 20 μl/min for 1 min.

Optimization of Assay Conditions: We first optimized the concentration of capture antibody by immobilizing different concentration of anti-cTnI capture antibody for assay of Troponin I in spiked plasma samples with a high concentration (10 ng/mL), low concentration (0.1 ng/mL), and blank (0 ng/mL), of troponin I. As shown in FIG. 27A, the signal for 10 ng/mL of cTnI increases with an increase in the concentration of capture antibody from 50 pg/mL to 500 μg/mL. With further increase in the concentration, the signal for 10 ng/ml decrease which may be due to crowding or protein-protein interaction. Signal for 0.1 ng/ml was only observed at concentrations higher than 500 μg/mL. 500 μg/mL also showed the highest signal to noise ratio, so it was considered the optimum concentration, and further experiments were performed with capture antibody concentration of 500 μg/mL. Similarly, 5 μg/mL of detection antibody that showed the highest signal to noise ratio with no background (0 ng/mL) signal was considered as the optimum concentration of anti-cTnI detection antibody and used for further experiments (FIG. 27B). FIG. 27C shows the optimization of the concentration of Streptavidin-poly HRP as 5 μg/mL and also shows that higher concentrations of HRP may lead to a thick layer of TMB which can have an insulating effect ultimately decreasing the signal. To complete the assay optimization of Troponin I, the time for precipitation of TMB was optimized. An increase in precipitation time for TMB can increase the resulting signal ultimately increasing the sensitivity of the assay but over precipitation of TMB can lead to an insulating effect decreasing the output signal. As seen from FIG. 27D, 2 min of TMB precipitation time was considered as optimum TMB precipitation time as it could detect lower concentrations of cTnITC (0.05 ng/mL and 0.01 ng/mL) where 1 min did not show any signal.

As the ultimate aim of the EC-Biosensor was to perform multiplex detection, the concentration of Streptavidin-poly HRP and TMB precipitation time was kept consistent and the concentration of detection antibody was optimized for each of the other biomarkers. FIG. 27E shows the optimization of the BNP detection antibody as 15 μg/mL as it gave the highest signal to noise ratio without any background noise (0 ng/mL). Likewise, FIG. 27F shows the optimization of NT-proBNP detection antibody as 6 μg/mL. The concentration of detection antibody higher than 6 μg/mL showed background signal. Finally, 6 μg/mL was considered as optimum cTnITC detection antibody concentration as shown in FIG. 27G.

Specificity of MI and TBI Biomarkers in 96 well assay: Specificity and cross-reactivity for the assay were tested in the buffer in 96 well plate. FIG. 27A shows specific signal for BNP as increasing intensity of signal with increasing concentration of BNP (black dots). Even a high concentration of 10 ng/mL of BNP showed a similar signal as 0 ng/mL of BNP with all other non-specific capture or detection antibody which shows that there was no non-specific binding with capture or detection antibody for (cTnI, NT-proBNP, cTnITC, GFAP, & S100b). FIG. 33A shows specificity of NT-proBNP and similar to BNP it only shows the specific signal for NT-proBNP and all other non-specific capture and detection antibody shows signal similar to 0 ng/mL even at high concentrations. Similarly, FIGS. 33B and 33C show the specificity test for GFAP and S100b where only the specific signal can be observed. All the non-specific capture and detection antibodies showed signals similar to 0 ng/mL showing there is no cross-reactivity or non-specific binding.

As seen in FIG. 34A abcam anti-cTnI could only detect up to 10 ng/ml of cTnITC, rest concentration showed similar a signal with non-specific capture/detection antibody as abcam antibody has epitope against cTnI and not cTnITC complex. FIG. 34B shows abcam anti-cTnI antibody pair can specifically detect cTnI but has high cross-reactivity with BNP and NT-proBNP capture antibody and thus cannot be used in a multiplex setting with those biomarkers. As the antibody pair from Advanced Immunochemical Inc. (AIC) had an epitope against cTnITC complex, the sensitivity was very less against cTnI as shown in FIG. 34D. Finally, FIG. 34C shows a specific signal for cTnITC with the increase in signal from 0 ng/mL to 10 ng/mL. The non-specific signal was very minimum thus AIC antibody pair and cTnITC will be used for all further multiplex detection. Based on these specificity and cross-reactivity tests, all these biomarkers (BNP, NT-proBNP, cTnITC, GFAP, and S100b) can be used for multiplexed detection.

### Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

As used herein the term “comprising” or “comprises” is used in reference to compositions, methods, and respective component(s) thereof, that are essential to the claimed invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the claimed invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise.

Other than in the operating examples, or where otherwise indicated, all numbers expressing quantities of ingredients or reaction conditions used herein should be understood as modified in all instances by the term “about.” The term “about” when used in connection with percentages may mean±5% (e.g., ±4%, ±3%, ±2%, ±1%) of the value being referred to.

Where a range of values is provided, each numerical value between the upper and lower limits of the range is contemplated and disclosed herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents, patent applications, and publications identified are expressly incorporated herein by reference for the purpose of describing and disclosing, for example, the methodologies described in such publications that can be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method for making a coating on a surface of a substrate, the method comprising applying a mixture to a surface of a substrate while maintaining the substrate at an elevated temperature, wherein the mixture comprises a particulate material and a proteinaceous material.

2. The method of claim 1, wherein the mixture further comprises a cross-linking agent.

3. The method of claim 1, wherein the proteinaceous material includes a cross linking agent attached to or as part of the proteinaceous material's structure.

4. The method of claim 1, wherein the elevated temperature is maintained for at least 10 seconds and less than two minutes.

5. The method of claim 1, wherein the elevated temperature is at least 50° C.

6. The method of claim 1, wherein the method further comprises denaturing the proteinaceous material.

7. The method of claim 6, wherein said denaturing the proteinaceous material is after applying the mixture to the substrate.

8. The method of claim 1, wherein the substrate is a particle, a nano-particle, a micro-particle, a nano-fiber, a micro-fiber, a flake, a chip, a crystal, a porous substrate, a wafer, a wire, a nano-wire, a micro-wire, a channel, a nano-channel, a micro-channel, a rod, a nano-rod, a micro-rod, a foil, a sheet, a web, or combination of these forms.

9. The method of claim 1, wherein the substrate comprise a material selected from the group consisting of metals, polymers, carbon based materials, ceramics, glass and any combinations thereof.

10. The method of claim 9, wherein the substrate comprises gold.

11. The method of claim 9, wherein the substrate comprises graphite, diamond, glassy carbon, or carbon nano-tubes.

12. The method of claim 9, wherein the substrate includes an organic polymer.

13. The method of claim 1, wherein the particulate material is a rod, fiber, a particle, a flake or combinations of these.

14. The method of claim 1, wherein the particulate material is a dielectric.

15. The method of claim 1, wherein the particulate material is a conductor or semi-conductor.

16. The method of claim 1, wherein the particulate material comprises an allotrope of carbon atoms arranged in a hexagonal lattice.

17. The method of claim 1, further comprising a step of pre-treating the substrate prior to applying the mixture.

18. The method of claim 1, wherein applying the mixture comprises spraying, spin coating, dip coating, inkjet printing, vapor deposition, 3-D printing, painting, drop casting or any combination thereof.

19. The method of claim 1, wherein the method is a continuous process or semi-continuous process, optionally said semi-continuous process is reel to reel or pattern deposition on a wafer.

20. The method of claim 1, wherein the method further comprises a step of denaturing the proteinaceous material and subsequently adding a temperature sensitive material to the mixture prior to coating the substrate.

21. The method of claim 1, wherein the substrate surface defines a channel or chamber, optionally, the substrate surface defines a channel in a microfluidic device or the substrate surface defines a chamber in a microfluidic device.

22. The method of claim 1, wherein the substrate is a micro/nano gap devices where the coating provides higher sensitivity and the coating can either be used to coat the surfaces of a gap in the micro/nano gap device, or the coating is applied for surface modification to enable linking of specific probes and antifouling properties.

23. The method of claim 1, further comprising applying a layer of a second substrate on the coating of proteinaceous material and optionally coating a second mixture comprising a second mixture and second proteinaceous material on the second substrate, providing a layered material having alternating layers of substrate and proteinaceous/particulate material.

24. A substrate comprising a coating on a surface thereof, wherein said coating is applied using a method of any one of claims 1-23.

25. The substrate of claim 24, wherein the substrate is an electrode, a capacitor, a bio-Field Effect transistor (bio-FET), transistors, and optical devices.

26. A capacitor comprising a dielectric material dispersed in a denatured and cross-linked proteinaceous material, and covering a conductive substrate.

27. A bio-FET comprising a composition including a particulate material dispersed in a denatured proteinaceous material and coating at least a portion of a transistor.

28. The bio-FET according to claim 27, wherein the compositing is coated on a gate of the bio-FET.
